(19) United States
(12) Patent Application Publication
(10) Pub. No.: US 2026/0306247 A1
(43) Pub. Date: Oct. 8, 2026

(54) SYSTEMS AND METHODS FOR CUSTOM 3D PRINTING OF PROSTHETIC EYES WITH ENHANCED FUNCTIONAL AND AESTHETIC FEATURES

(71) Applicant: Atheneum Optical Sciences, LLC, Orange Park, FL (US)

(72) Inventors: W. Anthony Martin, Orange Park, FL (US); Randall Pugh, Jacksonville, FL (US); Genesh Narayanan Kumar, Ponte Vedra Beach, FL (US); Christopher Wildsmith, Jacksonville, FL (US)

(73) Assignee: Atheneum Optical Sciences, LLC, Orange Park, FL (US)

(21) Appl. No.: 19/640,902

(22) Filed: Apr. 7, 2026

Related U.S. Application Data
(60) Provisional application No. 63/784,843, filed on Apr. 7, 2025.

(51) Int. Cl. A61F 2/14; B33Y 10/00; B33Y 50/00; B33Y 80/00

(52) U.S. Cl. CPC A61F 2/141; B33Y 50/00; B33Y 80/00; A61F 2240/002; B33Y 10/00

(57) ABSTRACT

A prosthetic eye or aesthetic contact lens and methods of manufacture of the same using additive manufacturing. Methods may include scanning a functional eye of a user to generate a prosthetic eye model matching the coloring and sizing features of the scanned eye. A substrate is positioned relative to one or more print heads, which deposit polymerizable mixtures as deposited droplets to form the sclera portion, vein pattern, and iris structure. A first polymerizable mixture may replicate a natural sclera, a second polymerizable mixture may form natural features, such as vein patterns, and a third polymerizable mixture may create an iris with a pupil region. The prosthetic eye may include an internal cavity filled with a liquid solution to allow iris movement, liquid pouches for hydration or medication, and integrated electronic components such as cameras, sensors, and wireless communication modules.

### CROSS-REFERENCE TO RELATED APPLICATIONS

This application claims priority benefit to U.S. Provisional Application Ser. No. 63/784,843 filed Apr. 7, 2025, and entitled “SYSTEMS AND METHODS FOR CUSTOM 3D PRINTING OF PROSTHETIC EYES WITH ENHANCED FUNCTIONAL AND AESTHETIC FEATURES,” and U.S. Provisional Application Ser. No. 64/027,679 filed Apr. 3, 2026, and entitled “SYSTEMS AND METHODS FOR CUSTOM 3D PRINTING OF PROSTHETIC EYES WITH ENHANCED FUNCTIONAL AND AESTHETIC FEATURES”; the entireties of which are incorporated herein at least by virtue of this reference.

### FIELD OF THE INVENTION

The present invention relates to prosthetic eyes and methods for manufacturing the same using additive manufacturing techniques, including 3D printing and multi-material deposition processes. More particularly, the invention pertains to a prosthetic eye with enhanced aesthetic realism, functional adaptability, and embedded technological features. The invention further relates to an implantable prosthetic system for users with damaged or missing eyes, providing both cosmetic and functional improvements.

### BACKGROUND OF THE INVENTION

Prosthetic eyes have been used for centuries to address the cosmetic needs of individuals who have lost one or both eyes due to trauma, disease, or congenital defects. Prosthetic eyes, also known as ocular prostheses, have been developed to replace missing or damaged eyes, primarily for cosmetic purposes. These devices serve to restore the appearance of a natural eye, improve self-esteem, and provide psychological benefits to individuals who have experienced eye loss due to trauma, disease, or congenital conditions. Traditional methods for manufacturing prosthetic eyes involve labor-intensive and time-consuming processes, such as hand painting and manual fitting, which can result in variability in quality and user satisfaction.

The traditional process for creating prosthetic eyes typically begins with an impression of the eye socket. This step involves inserting a mold material into the socket to capture its unique contours. A wax model is then created from the impression and manually sculpted to fit the socket's shape. Once the wax model is refined, a technician hand-paints the prosthetic to mimic the color, texture, and appearance of the functional eye. This process often requires multiple iterations and adjustments to ensure the prosthetic is comfortable and aesthetically accurate. While this method has been widely used, it poses several challenges that limit its efficiency and precision.

One of the primary drawbacks of traditional methods is the reliance on skilled artisans to hand-paint and manually shape the prosthetic eye. The availability of such artisans is limited, and the process is inherently subjective, leading to variability in the final product. This reliance on manual labor not only increases production costs but also extends the time required to create a prosthetic eye, often leading to delays for the user. Furthermore, achieving a high level of detail and symmetry with the functional eye can be challenging, resulting in prosthetics that may not fully meet the user's expectations.

Another significant limitation of current techniques is the lack of customization and adaptability. While traditional methods allow for some level of personalization, the process is largely static and does not account for changes in the user's eye socket over time. This can lead to discomfort or the need for frequent replacements and adjustments. Additionally, traditional prosthetic eyes are generally produced as standalone devices and do not integrate with modern technological advancements, such as digital imaging or data-driven customization.

Advances in 3D printing technology have opened new avenues for addressing the limitations of traditional prosthetic eye manufacturing. 3D printing, also known as additive manufacturing, enables the creation of highly detailed and precise objects by layering material in a controlled manner. This technology has already been applied in various medical fields, including orthopedics and dentistry, to produce customized implants, braces, and other devices. However, its application in the field of ocular prosthetics remains limited, despite its potential to revolutionize the manufacturing process.

Recent developments in optical scanning and imaging technologies have further enhanced the ability to capture detailed anatomical data. High-resolution 3D scanners can now generate accurate digital models of the user's functional eye, including its shape, size, and surface characteristics. These models can be combined with color-matching software to replicate the exact appearance of the functional eye. By integrating 3D scanning with 3D printing, it is possible to create prosthetic eyes that are not only highly customized but also more visually and functionally accurate than those produced using traditional methods.

Despite the potential of 3D printing and scanning technologies, several challenges remain unaddressed. For example, existing 3D printing techniques often face limitations in achieving the smooth, glossy finish required for a realistic prosthetic eye. Additionally, current materials used in 3D printing may lack the durability and biocompatibility necessary for prolonged use. Furthermore, integrating advanced features such as automated color matching and dynamic adjustments to account for changes in the user's eye socket remains a technical hurdle.

In addition to these functional and aesthetic limitations, the production of traditional prosthetic eyes remains a labor-intensive and time-consuming process. Handcrafting each prosthetic eye requires skilled artisans and can take weeks to complete, resulting in high costs and limited scalability. While 3D printing technologies have been introduced to address these challenges, their application in the field of ocular prosthetics remains underdeveloped, leaving significant room for innovation.

Therefore, there is a growing need for a comprehensive system that leverages 3D printing and scanning technologies to produce prosthetic eyes with a high degree of customization, precision, and aesthetic accuracy. Such a system would address the limitations of traditional methods by automating key steps, reducing production time, and enabling greater adaptability to the user's needs. By incorporating advanced imaging, material science, and manufacturing techniques, it is possible to create a next-generation solution for prosthetic eyes that meets the demands of modern users.

### SUMMARY OF THE INVENTION

In some embodiments, the present invention provides systems and methods for manufacturing a prosthetic eye, prosthetic lens, or portions thereof using additive manufacturing based on a digital model generated from scan data and/or image data of a desired eye. A scanning system may capture anatomical and optical details of a functional eye, including one or more of iris color and pattern, pupil size and location, scleral coloration, vein patterns, curvature, and dimensional features. The captured data may be processed into a prosthetic model, including a digital value pattern and/or an energy intensity pattern, for controlling one or more additive manufacturing printheads.

In some embodiments, one or more polymerizable mixtures are deposited as droplets onto a receiving surface and/or previously deposited material to form a prosthetic eye or prosthetic lens. A first polymerizable mixture may form a sclera portion, a second polymerizable mixture may form vein patterns, and a third polymerizable mixture may form an iris structure with a pupil region. Deposited material may be pinned to cause partial polymerization, and successive deposited volumes may meld and integrate prior to final cure. In some implementations, transparent polymerizable mixture may be deposited over pigmented portions to provide depth, gloss, and a natural appearance.

In some embodiments, the prosthetic eye may be formed directly on a prosthetic base or other substrate, including a pre-manufactured standard prosthetic base selected from a plurality of sizes and shapes. The additive manufacturing process may be performed with one printhead or multiple printheads, and may include controlled-atmosphere curing, multi-angle deposition, grayscale and/or digital value pattern control, and real-time adjustment based on imaging or sensor feedback. The resulting prosthetic eye may closely replicate the appearance of a patient's functional eye or may be customized based on another desired eye image.

In some embodiments, the prosthetic eye may further include enhanced functional features. Such features may include an internal cavity containing liquid and a movable iris structure to simulate natural eye motion, one or more liquid pouches configured to release hydration fluid or medication, one or more image-capturing devices, electronic circuitry and wireless communication components, physiological sensors, and/or coupling to an external assistive sensory device. In some embodiments, the prosthetic eye may be incorporated into an implant configured for attachment to a damaged eye portion of a user. This combination of scanning, digital modeling, additive manufacturing, and optional functional enhancements provides a highly customized prosthetic eye with improved realism, comfort, manufacturability, and adaptability.

### BRIEF DESCRIPTION OF THE DRAWINGS

FIGS. 1A-1B illustrate an exemplary process of scanning a functional eye of a user to create a prosthetic eye model, according to some embodiments of the present invention.

FIG. 1C illustrates an exemplary process for generating a digital value pattern from the scanned eye data for guiding the printing process, according to some embodiments of the present invention.

FIG. 2 illustrates an exemplary additive manufacturing apparatus and method that may be used in some implementations of the present invention.

FIG. 2A illustrates an exemplary printing process for printing a prosthetic eye on a substrate, including the printing of a prosthetic base and the iris, according to some embodiments of the present invention.

FIG. 2B illustrates an exemplary printing process for printing an iris and other patterns on a standard prosthetic base, according to some embodiments of the present invention.

FIG. 2C illustrates an exemplary printing process for printing a prosthetic eye layer-by-layer using patterned droplets, according to some embodiments of the present invention.

FIG. 3A is a schematic illustration of an alternative 3D printing apparatus according to some embodiments of the present invention.

FIG. 3B illustrates a schematic diagram of mechanisms to introduce tilt into an additive manufacturing system.

FIG. 3C illustrates a schematic diagram of a printhead movement relative to a receiving substrate.

FIG. 3D illustrates electro active devices for providing camber movement to one or both of a printhead and a receiving substrate.

FIGS. 4A, 4B, and 4C are schematic illustrations of deposited droplets of polymerizable mixture integrating into a volume of polymerizable mixture previously deposited on a substrate.

FIG. 4D illustrates an exemplary energy intensity pattern or map, represented as a grayscale image, which may be used to generate control protocols for 3D printing apparatus according to some embodiments of the present invention.

FIG. 4E illustrates a schematic diagram of an exemplary energy intensity pattern, represented as a grayscale image.

FIG. 4F is a schematic illustration of an exemplary change in shape of a droplet of polymerizable mixture after release from a printhead.

FIG. 5A illustrates an exemplary printing process for printing a prosthetic eye with components mimicking the natural motion of the iris, according to some embodiments of the present invention.

FIGS. 5B-5C illustrate iris movement within a cavity of the prosthetic eye, according to some embodiments of the present invention.

FIGS. 6A-6B illustrate an exemplary implant with a prosthetic eye, wherein the implant is attached to a damaged eye portion of a user, according to some embodiments of the present invention.

FIG. 7 describes method steps that may be executed while practicing some implementations of the present invention.

FIG. 7A illustrates additional method steps that may be executed in some implementations of the present invention.

FIG. 8 illustrates eye samples from various celebrities with respective prosthetic models generated through 3D printing for a user, according to some embodiments of the present invention.

FIG. 9 illustrates a prosthetic eye with an integrated camera, electronic circuitry, and sensors, enabling real-time image capture and wireless transmission to an external device.

FIG. 10 illustrates an exemplary prosthetic eye comprising liquid pouches for medication delivery, hydration, and a weeping imitation, according to some embodiments of the present invention.

FIGS. 11A-11B illustrate coupling of exemplary prosthetic eyes to an electronic sensory device, enabling blind users to sense visual signals transmitted from the prosthetic eyes using the electronic sensory device.

FIG. 12 illustrates exemplary steps of printing a prosthetic eye with an internal cavity, liquid pouches, and integrated electronic circuitries, according to some embodiments of the present invention.

FIGS. 13A-13C illustrate a flowchart of method steps that may be executed in some embodiments of the present invention.

### DETAILED DESCRIPTION OF THE INVENTION

According to the present invention, a prosthetic eye is manufactured using an additive manufacturing process based on a prosthetic eye model generated from a scan of a functional eye of a user. The scanned data provides a highly accurate template for replicating the natural eye's structure and appearance. The additive manufacturing process begins with positioning a substrate in a first position relative to one or more additive manufacturing print heads, which deposit polymerizable mixtures layer by layer or in droplets form to construct various components of the prosthetic eye. A first polymerizable mixture is used to form the sclera portion, mimicking the natural appearance of a human eye's sclera. This mixture may include embedded pigments or nanoparticles that precisely match the user's functional eye color. A second polymerizable mixture is deposited to create vein patterns, while a third polymerizable mixture is used to form an iris structure with a pupil region. The process involves integration of these deposited polymerizable mixtures to form a combined volume, followed by a pinning process that causes partial polymerization, allowing seamless melding of subsequent layers. The deposition, pinning, and polymerization steps are repeated iteratively to complete the prosthetic eye structure, and a final curing step solidifies the polymerized mixture into a stable form.

In some implementations, the first polymerizable mixture used to form the sclera portion includes pigments that closely replicate the texture, opacity, and color variations found in natural sclera tissue. The pigmentation may be dynamically controlled by an additive manufacturing system that adjusts pigment concentration in real time to provide accurate visual matching. Additionally, nanoparticles may be introduced into the polymerizable mixture to modify the mechanical properties of the sclera portion, such as increasing its strength, durability, or flexibility.

In certain embodiments, the iris structure of the prosthetic eye is printed using polymerizable mixtures with varying refractive indices to create a gradient color effect. This effect replicates the complex and multi-tonal nature of a natural iris, giving the prosthetic eye a more lifelike appearance. The refractive properties of the materials can be fine-tuned to interact with light in a manner that mimics natural iris reflections and depth perception. The printing process may involve controlled layering of transparent and pigmented materials to simulate the intricate fiber-like structure of a real human iris.

Some implementations may include the use of multiple print heads that deposit different polymerizable mixtures simultaneously. One print head may be dedicated to depositing the sclera-forming mixture, while additional print heads handle the vein pattern and iris structure deposition. Alternatively, a single print head may be used to sequentially deposit different polymerizable mixtures by switching between material reservoirs. This configuration allows for high precision in forming the prosthetic eye's anatomical features while maintaining a streamlined manufacturing process.

In some embodiments, the prosthetic eye is formed on a pre-manufactured standard prosthetic base, which serves as the substrate. This standard base may be selected from a set of predefined sizes and shapes, providing compatibility with a wide range of users. The additive manufacturing process then deposits the necessary polymerizable mixtures onto the standard base, modifying its surface to include user-specific details such as sclera coloration, vein patterns, and iris design.

In another approach, the substrate is placed on a conveyor belt system, which moves the substrate relative to the additive manufacturing print heads. This automated system allows for continuous, high-throughput manufacturing of prosthetic eyes, reducing production time while maintaining precision. The conveyor belt may reposition the substrate at various stages, allowing for sequential deposition of polymerizable mixtures, pinning, and curing.

Some implementations may involve repositioning the substrate at multiple positions relative to the print heads to facilitate multi-angle deposition. This repositioning may be achieved through robotic actuators, rotational mounts, or tilting platforms, enabling the print heads to apply polymerizable mixtures in precise orientations. The ability to reposition the substrate facilitates consistent deposition even in complex three-dimensional geometries.

In some embodiments, the prosthetic eye incorporates an internal cavity filled with a liquid solution to suspend the iris structure. This configuration allows the iris to exhibit slight movement in response to user motion, creating a dynamic effect that mimics natural eye movement. The liquid solution within the cavity may be a saline-based fluid or a polymeric suspension engineered for stability and controlled viscosity.

Some variants of the prosthetic eye further include one or more liquid pouches integrated within the sclera portion. These liquid pouches are configured to release hydration fluid or medication as needed. The hydration fluid may be beneficial for users experiencing dry eye conditions, while the medication delivery function allows for slow-release treatment of ocular conditions.

In certain embodiments, the liquid pouches are activated through user interaction. Blinking, pressing, or applying slight pressure to the prosthetic eye may trigger the controlled release of hydration fluid or medication. This mechanism allows the user to manage fluid release without the need for external intervention.

A specific implementation of the liquid pouches may involve creating a weeping effect in the prosthetic eye. This feature may particularly be useful for achieving a more lifelike appearance, as it allows the prosthetic eye to exhibit tear-like fluid release. The weeping effect may be triggered by increased pressure applied during moments of emotional response, closely resembling the natural behavior of human tear ducts.

In some embodiments, the prosthetic eye integrates one or more image-capturing devices within its structure. These image-capturing devices may be embedded in the iris or sclera region and are capable of capturing visual information from the user's surroundings.

Further embodiments include a communication module embedded within the prosthetic eye, allowing it to wirelessly transmit captured visual data to an external device such as a smartphone, computer, or assistive technology system. The communication module may use Bluetooth, Wi-Fi, or other wireless protocols to facilitate data transmission.

The prosthetic eye may also be configured to work with an Electronic sensory device, which is an assistive sensory substitution system for visually impaired users. The Electronic sensory device may be functional to receive visual data from the prosthetic eye's image-capturing device and convert it into electrical stimulation patterns that are transmitted to a nerve rich area of a user such as the tongue of the user. This stimulation provides sensory feedback, allowing the user to interpret visual information through touch perception.

Some embodiments further incorporate an AI engine in the external device receiving data from the prosthetic eye. The AI engine may analyze real-time visual input, detect objects, and identify faces using facial recognition. Upon detecting a recognized person, the AI engine may announce their name through an audio output device, helping the user recognize people in their vicinity.

In additional embodiments, the prosthetic eye includes one or more physiological sensors embedded within the structure. These sensors may measure parameters such as temperature, pressure, or moisture levels. The collected data can be used for health monitoring, detecting potential issues such as eye dryness, pressure imbalances, or inflammation.

Some implementations may involve different print heads managing the deposition of various polymerizable mixtures. One print head may exclusively deposit the first polymerizable mixture for the sclera, while other print heads handle the deposition of the vein pattern and iris structure. This division allows for precise control over each material's application, so that all components of the prosthetic eye maintain realistic detailing and durability.

In another approach, a single print head sequentially deposits different polymerizable mixtures by dynamically switching between material sources. This method streamlines the manufacturing process while maintaining high precision.

In some variants, the prosthetic eye's polymerizable mixtures may include CMYK-based pigment systems to achieve a full-color representation of natural eye components. By carefully layering these pigments, a highly realistic iris and sclera coloration may be achieved.

Other embodiments incorporate a transparent polymerizable mixture that is intermittently deposited during the printing process. This transparent material enhances the three-dimensional effect of the vein pattern and iris structure by creating depth through refractive layering.

In some embodiments of the invention, a prosthetic eye is manufactured using an additive manufacturing process that replicates the anatomical features of a natural human eye. The prosthetic base comprises a sclera portion, which may be printed using a first polymerizable mixture deposited in droplets from one or more print heads. The deposition process may be executed with precise control over material distribution to replicate the natural texture, opacity, and subtle variations present in the sclera of a human eye. The first polymerizable mixture may include embedded pigments or nanoparticle dispersions that contribute to the natural color characteristics of the sclera, so that the final prosthetic eye closely resembles the user's functional eye. The sclera portion serves as a foundation upon which additional anatomical details, including the vein pattern and iris structure, are added using subsequent depositions of polymerizable mixtures.

In some variations, the prosthetic eye incorporates a vein pattern integrated within the sclera portion. The vein pattern may be printed using a second polymerizable mixture, which is deposited from one or more print heads in a predefined pattern of droplets. The pattern mimics the natural vascular structure observed in a human eye, adding to the overall realism of the prosthetic eye. To achieve a lifelike appearance, the second polymerizable mixture may include CMYK pigments, which enable high-precision color variations to resemble natural blood vessels. The vein pattern can be designed using computational modeling based on high-resolution scans of real human eyes, providing an anatomically accurate and randomized vein network that enhances the authenticity of the prosthetic eye.

In further implementations, the iris structure comprises a pupil region and may be printed using a third polymerizable mixture, which is deposited from the print heads in controlled droplet patterns. The iris structure may be generated based on a user's functional eye scan or designed according to an aesthetic preference. For example, some users may opt for an iris model derived from celebrity eye samples or fictional character designs. The third polymerizable mixture used for the iris may also contain CMYK pigments that are deposited in varying droplet densities to create a gradient effect, simulating the intricate radial fiber patterns and depth variations characteristic of a real iris. To enhance depth perception and optical realism, the CMYK pigments may be combined with translucent or pearlescent additives, allowing light to interact with the printed iris structure in a natural way.

In some embodiments, a single print head deposits the first polymerizable mixture for the sclera portion, the second polymerizable mixture for the vein pattern, and the third polymerizable mixture for the iris structure. The single print head is capable of dynamically switching between different material reservoirs, providing precise control over deposition rates and material transitions. Alternatively, multiple print heads may be utilized to deposit each polymerizable mixture independently, allowing for parallel processing and increased efficiency in the prosthetic eye fabrication. For example, a first print head may deposit the first polymerizable mixture forming the sclera portion, while a second print head deposits the second polymerizable mixture forming the vein pattern, and a third print head prints the third polymerizable mixture forming the iris structure.

In other configurations, the prosthetic eye model may be created based on a digital reconstruction of an eye sample belonging to a celebrity or fictional character. The process involves collecting high-resolution eye images from publicly available sources, processing the images to extract detailed iris and sclera patterns, and generating a 3D prosthetic eye model for printing. This implementation allows users to select a prosthetic eye design based on their personal preferences, enabling customized aesthetic enhancements. Further, the desired eye sample on which the digital reconstruction is performed may be that an individual patient. For example, it may be desirable to create a prosthetic eye for a patient that substantially matches, in terms of iris color and configuration, pupil size and location, and optionally sclera shape and location etc., the other eye of the patient. High resolution eye images of the patient's “good” or desired eye are captured in order to match anatomical details of that eye (i.e., iris design and color, pupil size and location, sclera properties etc.) on the prosthetic eye. If shape and dimensions of the eye needing a prosthetic are not too different from the good or desired eye, the corneal and scleral topographies of the good or desired eye may be used in the digital model for the desired prosthetic lens. If significantly different, then the corneal and scleral topographies of the eye needing a prosthetic can be captured and used to improve the fit of the new lens.

In certain implementations, the prosthetic eye includes an internal cavity that houses a suspended iris structure. The internal cavity is filled with a liquid solution, which allows the iris to move slightly relative to the sclera portion, creating a dynamic eye movement effect. The liquid solution may be a saline-based fluid or a polymeric suspension engineered to maintain stability while allowing smooth motion of the iris. This configuration closely resembles the natural mobility of a human iris, improving both the aesthetic and functional aspects of the prosthetic eye.

Some embodiments further integrate an image-capturing device within the prosthetic eye, positioned either within the sclera portion or the iris structure. The image-capturing device is configured to capture visual data from the surroundings of the wearer. In some implementations, the prosthetic eye features both a front-facing camera aligned with the iris structure to capture a direct field of view and a side-facing camera positioned at an angle to capture peripheral visual data. The combination of multiple cameras provides a comprehensive visual feed, which can be processed for various assistive applications.

To facilitate data processing and transmission, an electronic circuitry is embedded within the prosthetic base. The electronic circuitry is responsible for managing image data, controlling embedded sensors, and wirelessly transmitting collected information to external devices. The communication module within the electronic circuitry supports various wireless communication protocols such as Bluetooth, Wi-Fi, or near-field communication (NFC), facilitating secure and efficient data exchange. The prosthetic eye can transmit captured visual data to an external device, such as a smartphone, where the data can be analyzed, stored, or further processed.

In further embodiments, the prosthetic eye is communicatively coupled to an Electronic sensory device, which is configured to convert visual data into electrical stimulation patterns. These stimulation patterns are delivered to the tongue of the wearer, allowing the user to perceive visual information through tongue-based sensory feedback. The Electronic sensory device enhances accessibility for blind or visually impaired users, providing an alternative means of understanding their surroundings.

To improve wearer comfort and health monitoring, the prosthetic eye may be equipped with physiological sensors embedded within the sclera portion or electronic circuitry. These sensors are configured to measure parameters such as temperature, pressure, or moisture levels, providing real-time feedback on ocular conditions. The collected sensor data can be transmitted to an external device, allowing users or healthcare professionals to monitor eye health and detect potential abnormalities.

In some embodiments, the prosthetic eye is integrated within an implant that is attachable to a damaged eye portion of a user. The implant serves as a structural interface for securely positioning the prosthetic eye within the user's eye socket. The implant may be customized based on medical imaging scans to provide an optimal fit, providing stability and comfort for long-term wear.

In further implementations, the CMYK pigments used for printing the vein pattern and iris structure are deposited in micro-droplet formations, precisely controlling the depth and thickness of each layer and component (sclera, iris, pupil, and vein pattern). This technique enhances the visibility of intricate details, allowing the vein pattern to appear three-dimensional when viewed from different angles. Additionally, a transparent polymerizable mixture may be intermittently deposited in combination with the other polymerizable mixtures, forming a layered refractive structure that further enhances depth perception. This transparent material interacts with light in a way that amplifies the naturalistic appearance of the prosthetic eye, facilitating a visually seamless integration with the wearer's functional eye.

Polymerizable mixture that has been pinned may act as a subsequent receiving surface and receive additional polymerizable mixture applied in a pattern of a greyscale image or digital value pattern. After a final application of polymerizable mixture, the polymerizable mixture accumulated on the receiving surface may be exposed to sufficient actinic conditions, (e.g., radiation and thermal energy) to cure the accumulated polymerizable mixture into a polymer.

An atmosphere encompassing the droplets of polymerizable mixture during application onto the receiving surface and during pendency on the receiving surface prior to cure, may be carefully controlled in order to achieve a consistent optical quality of a device formed by the cured polymerizable mixture.

### Glossary

In this description and claims directed to the presented invention, various terms may be used for which the following definitions will apply:

“Actinic Radiation” as used herein, refers to the emission of energy that is capable of initiating a chemical reaction in an associated Polymerizable Mixture. In some embodiments, actinic radiation includes radiation with a wavelength of energy in a range of 280-450 nm. In some more specific examples embodiments, an actinic radiation corresponding to UVA and blue light includes an energy with a wavelength in the range of 315-450 nm, some preferred embodiments include energy in the 365 nm to 400 nm range.

“Addition Based Manufacture” (sometimes referred to herein as “additive manufacturing” means a process during which units of material are added to a structure being formed via the aggregation of the units of material into a shape.

“Arcuate” as used herein, refers to a geometric shape including a curved portion.

“Cure” as used herein refers to exposing a polymerizable mixture to actinic conditions which may include Fixing Radiation and/or thermal energy of sufficient intensity and for a sufficient duration of time to crosslink a majority of Polymerizable mixture.

“Fixing Radiation” as used herein, refers to Actinic Radiation of appropriate wavelength, and sufficient intensity and duration to crosslink a majority of Polymerizable mixture exposed to the Fixing Radiation.

“Gelling” or “Gelation” refers to a degree of polymerization sufficient to stop or substantially slow the movement of polymerizable mixture deposited on a receiving surface while allowing subsequent droplets to meld or otherwise integrate with previously deposited polymerizable mixture and form a structure with a single mass of polymerizable mixture without distortion. Gelled polymerizable mixture moves to a higher viscosity state, but stops short of full cure. Pinning or gelation (or gelling) enhances the management of flow and form, and provides a high quality surface.

“Gel Point” as used herein shall refer to a point in a polymerization process at which a gel or insoluble fraction is formed. Gel point may be considered the extent of conversion at which the liquid polymerization mixture becomes a high viscous material that is immobile on a stationary surface. Gel point can be determined, for example, using a Soxhlet experiment: Polymer reaction is stopped at different time points and a resulting polymer is analyzed to determine a weight fraction of residual insoluble polymer. The data can be extrapolated to a point where no gel is yet present. This point, where some polymer reaction has occurred, but no gel is present, is the gel point. The gel point may also be determined by analyzing a viscosity of a polymerizable mixture during a reaction. The viscosity can be measured using a parallel plate rheometer, with the polymerizable mixture between the plates. At least one plate should be transparent to radiation at the wavelength used for polymerization. The point at which the viscosity approaches infinity is the gel point. Gel point may generally be considered to occur at the same degree of conversion for a given polymer system and specified reaction conditions.

“Inhibitor” as used herein refers to a chemical reactant or process that slows or halts a chemical reaction.

“Initiator” as used herein refers to a substance that initiates a chain reaction or polymerization.

“Intensity” as used herein refers to an amount of power transferred per unit area, where the area is measured on a plane perpendicular to a direction of propagation of the energy (e.g., watts per square meter (W/m²)).

“Meld” (sometimes referred to as integrate) as used herein means to merge together droplets of polymerizable mixture from two or more depositions so that they become a single item with polymer chains that include polymerizable mixture from a first deposition and polymerizable mixture from a second deposition.

“Optical” as used herein refers to a characteristic modifying or relating to a behavior or property of light.

“Optical Interface” as used herein refers to an object, or portion of an object that affects a behavior or property of light.

“Pinning” as used herein refers to the application of actinic conditions, such as exposure to limited actinic radiation, to a polymerizable mixture in an amount sufficient to perform a gelation process or gelling, but not cause the polymerizable mixture to cure.

“Polymerizable Mixture” (sometimes referred to as “PM” or “polymerizable mixture”) as used herein, refers to a liquid mixture of components (reactive and possibly also non-reactive components) which upon exposure to an external energy (e.g., actinic radiation in a range of 280-450 nm (e.g., UV-light or blue light or heat) is capable of undergoing polymerization to form a polymer or polymer network. A polymerizable mixture may include a monomer or prepolymer material which can be cured and/or crosslinked to form a prosthetic eye or modify an existing prosthetic eye. Various embodiments can include Polymerizable Mixtures with one or more additives such as: UV blockers, bonding agents, tints, photo initiators or catalysts, and other additives one might desire in prosthetic eyes. In some embodiments, a Polymerizable Mixture may also be a Hydrogel Precursor.

When used herein, the expression oxygen equilibrium concentration of the polymerizable mixture of X is intended to mean the oxygen concentration in the polymerizable mixture obtained if the mixture hypothetically is allowed to equilibrate at 1.0 atmospheres (1013 millibar) with an atmosphere having an oxygen concentration of X %.

The term hydrogel refers to crosslinked polymers which have absorbed water (swelled) to a water content of at least 10 weight-% thereof. Preferably such hydrogel materials have a water content of at least 20 weight-%, such as at least 25 weight-%, and up to 70 to 90 weight-%.

When used herein, the term polymerizable mixture refers to a liquid mixture of components (reactive and possibly also non-reactive components) which upon exposure to an external energy (e.g., actinic radiation 280-450 nm (like UV-light or blue light) or heat) is capable of undergoing polymerization to form a polymer or polymer network. Typically, the mixture comprises reactive components such as monomers, macromers, prepolymers, cross-linkers, and initiators. Moreover, polymerizable mixture may further include other ingredients such as, by way of non-limiting example, one or more of: additives such as wetting agents, release agents, dyes, light absorbing compounds such as UV absorbers and photochromic compounds, any of which may be reactive or non-reactive but are capable of being retained within the resulting prosthetic eye, as well as one or more: pharmaceuticals, vitamins, antioxidants, and nutraceutical compounds. It will be appreciated that a wide range of additives may be added based upon the prosthetic eye, how the prosthetic eye is made, and the prosthetic eye's intended use.

A mixture may be considered a polymerizable mixture if one or more constituents of the mixture (such as, for example, monomer, macromers, prepolymers, crosslinkers, etc.) includes at least one polymerizable functional group, such as an ethylenically unsaturated group, such as, by way of non-limiting example: (meth)acrylate, (meth)acrylamide, vinyl, N-vinyl lactam, N-vinylamide, and styryl functional groups.

In some embodiments, a Polymerizable Mixture contains at least one hydrophilic component. In some embodiments, hydrophilic components included in a Polymerizable Mixture may be selected from the hydrophilic monomers, e.g., those known to be useful to prepare hydrogels.

In some exemplary and specific embodiments, hydrophilic means that at least 5 grams of a compound(s) are soluble in 100 mL of deionized water at 25 °C under weakly acidic (pH between 5 and 7) or basic conditions (pH from 7 to 9), and in some embodiments 10 grams of the compound(s) are soluble in 100 mL of deionized water at 25 °C under weakly acidic or basic conditions. Other embodiments are also within the scope of the invention. In addition, in other uses the “hydrophilic” may be ascertained by methods other than solubility, and the term may be used as a condition and/or a modifier.

In some exemplary and specific embodiments, “hydrophobic” means that 5 grams of hydrophobic compound does not fully dissolve in 100 mL of deionized water at 25 °C under weakly acidic or basic conditions. The solubility of the compounds can be confirmed by visual observation, with any visible precipitants or turbidity indicating that the compound is hydrophobic. Solubility may be determined, for example, in some embodiments, after about eight hours of mixing or stirring. Other embodiments are also within the scope of the invention. In addition, in other uses the condition of “hydrophobic” may be ascertained by methods other than solubility, and the term may be used as a condition and/or a modifier.

One class of suitable hydrophilic monomers includes acrylic-or vinyl-containing monomers. Such hydrophilic monomers may themselves be used as crosslinking agents, however, where hydrophilic monomers having more than one polymerizable functional group are used, their concentration should be limited as discussed above to provide a prosthetic eye having the desired features.

The term vinyl-type or vinyl-containing monomers refers to monomers containing the vinyl grouping (—CH═CH2) and that are capable of polymerizing. Examples of hydrophilic vinyl-containing monomers include, but are not limited to, monomers such as N-vinyl amides, N-vinyl lactams (e.g., N-vinylpyrrolidone (NVP) ), N-vinyl-N-methyl acetamide, N-vinyl-N-ethyl acetamide, and N-vinyl-N-ethyl formamide, N-vinyl formamide. Alternative vinyl-containing monomers include, but are not limited to, 1-methyl-3-methylene-2-pyrrolidone, 1-methyl-5-methylene-2-pyrrolidone, and 5-methyl-3-methylene-2-pyrrolidone.

Acrylic-type or acrylic-containing monomers are those monomers containing the acrylic group: (CH2═CRCOX) wherein R is H or CH3, and X is O or N, which are also known to polymerize readily, such as N,N-dimethyl acrylamide (DMA) , 2-hydroxyethyl methacrylate (HEMA) , glycerol methacrylate, 2-hydroxyethyl methacrylamide, polyethyleneglycol monomethacrylate, methacrylic acid, and mixtures thereof.

Other hydrophilic monomers that can be employed in the invention include, but are not limited to, polyoxyethylene polyols having one or more of the terminal hydroxyl groups replaced with a functional group containing a polymerizable double bond. Examples include polyethylene glycol, ethoxylated C1-20 alkyl glucosides, and ethoxylated bisphenol A reacted with one or more molar equivalents of an end-capping group such as isocyanatoethyl methacrylate, methacrylic anhydride, methacryloyl chloride, vinylbenzoyl chloride, or the like, to produce a polyethylene polyol having one or more terminal polymerizable olefinic groups bonded to the polyethylene polyol through linking moieties such as carbamate or ester groups. Other suitable hydrophilic monomers will be apparent to one skilled in the art.

In some exemplary and nonlimiting embodiments, the hydrophilic component comprises at least one hydrophilic monomer such as DMA, HEMA, glycerol methacrylate, 2-hydroxyethyl methacrylamide, NVP, N-vinyl-N-methyl acrylamide, polyethyleneglycol monomethacrylate, and combinations thereof. In another embodiment, the hydrophilic monomers comprise at least one of DMA, HEMA, NVP and N-vinyl-N-methyl acrylamide and mixtures thereof. In another embodiment, the hydrophilic monomer comprises DMA and/or HEMA.

Hydrophilic component(s) (e.g., hydrophilic monomer(s)) may be present in a wide range of amounts, depending upon the specific balance of properties desired. In some embodiments, the amount of the hydrophilic component is up to 60 weight-%, such as from 5 to 40 weight-% based upon all reactive components.

Hydrophobic silicone-containing components (or silicone components) may be considered those that contain at least one [—Si—O—Si] group in a monomer, macromer or prepolymer. In some embodiments, the Si and attached O are present in the silicone-containing component in an amount greater than 20 weight percent, such as greater than 30 weight percent of the total molecular weight of the silicone-containing component. Useful silicone-containing components include polymerizable functional groups such as acrylate, methacrylate, acrylamide, methacrylamide, N-vinyl lactam, N-vinylamide, and styryl functional groups.

Also, in some exemplary and non-limiting embodiments, cross-linking monomers may be employed, either singly or in combination, and may include ethylene glycol dimethacrylate, trimethylolpropane trimethacrylate, glycerol trimethacrylate, polyethylene glycol dimethacrylate (wherein the polyethylene glycol has a molecular weight up to, e.g., 400), and other polyacrylate and polymethacrylate esters. The cross-linking monomer may be used in the usual amounts, e.g., from 0.1 to 5, and preferably in amounts of from 0.2 to 3, parts by weight per 100 parts by weight of the polymerizable mixture.

Another monomer that may also be used in some exemplary and nonlimiting embodiments, includes methacrylic acid, which may be used to influence an amount of water that a hydrogel may absorb at equilibrium. Methacrylic acid is usually employed in amounts of from 0.2 to 8 parts by weight per 100 parts of the hydrophilic monomers like HEMA. Other monomers that can be present in the polymerization mixture include methoxyethyl methacrylate, acrylic acid, and the like.

In some exemplary and nonlimiting embodiments, the polymerizable mixture comprises hydroxyethyl methacrylate (HEMA) or hydroxyethyl acrylate (HEA) monomers, preferably hydroxyethyl methacrylate (HEMA) monomers.

In some exemplary and nonlimiting embodiments, the polymerizable mixture comprises methacrylate or acrylate monomers not being hydroxyethyl methacrylate or hydroxyethyl acrylate monomers.

In some exemplary and nonlimiting embodiments, the polymerizable mixture comprises reactive silicone monomers or oligomers.

In some further exemplary and non-limiting embodiments, the polymerizable mixture after polymerization may provide a polymer which is non-swellable in water, e.g., a polymer that is not able to take up a water content of more than 2 weight-%.

One or more polymerization initiators may be included in the polymerizable mixture. Examples of polymerization initiators include, but are not limited to, compounds such as lauryl peroxide, benzoyl peroxide, isopropyl percarbonate, azobisisobutyronitrile, and the like, which generate free radicals at moderately elevated temperatures, and photo-initiator systems such as aromatic alpha-hydroxy ketones, alkoxyoxybenzoins, acetophenones, acylphosphine oxides, bisacylphosphine oxides, and a tertiary amine plus a diketone, mixtures thereof and the like. Illustrative examples of photo-initiators are 1-hydroxycyclohexyl phenyl ketone, 2-hydroxy-2-methyl-1-phenyl-propan-1-one, bis(2,6-dimethoxybenzoyl)-2,4-4-trimethylpentyl phosphine oxide (DMBAPO), bis(2,4,6-trimethylbenzoyl)-phenyl phosphineoxide (Irgacure 819), 2,4,6-trimethylbenzyldiphenyl phosphine oxide and 2,4,6-trimethylbenzoyl diphenylphosphine oxide, benzoin methyl ester and a combination of camphorquinone and ethyl 4-(N,N-dimethylamino)benzoate. Commercially available ultra-violet and visible light initiator systems include, but are not limited to, Irgacure 819® and Irgacure 1700® (from Ciba Specialty Chemicals) and Lucirin TPO initiator (available from BASF). Commercially available UV photo-initiators include Irgacure 651, Darocur 1173 and Darocur 2959 (Ciba Specialty Chemicals). These and other photo-initiators which may be used are disclosed in Volume III, Photoinitiators for Free Radical Cationic & Anionic Photopolymerization, 2nd Edition by J. V. Crivello & K. Dietliker; edited by G. Bradley; John Wiley and Sons; New York; 1998.

In some embodiments, a polymerization initiator is included in a polymerizable mixture in amounts capable of initiating polymerization of the polymerizable mixture, such as 0.1 to 2 weight-%. Polymerization of the polymerizable mixture can be initiated using the appropriate choice of heat or visible or ultraviolet light, or other energy, depending upon a polymerization initiator used. Alternatively, in some embodiments, initiation can be conducted without a photo-initiator using, for example, e-beam. However, when a photo-initiator is used, the preferred initiators are bisacylphosphine oxides, such as bis(2,4,6-trimethylbenzoyl)-phenyl phosphine oxide (Irgacure 819®) or a combination of 1-hydroxycyclohexyl phenyl ketone and DMBAPO, and in another embodiment the method of polymerization initiation is via visible light activation.

In some embodiments, a polymerizable mixture may include one or more internal wetting agents. Internal wetting agents may include, but are not limited to, high molecular weight, hydrophilic polymers. Examples of internal wetting agents include, but are not limited to, polyamides such as poly(N-vinyl pyrrolidone) and poly(N-vinyl-N-methyl acetamide).

The internal wetting agent(s) may be present in a wide range of amounts, depending upon the specific parameter desired. In some embodiments, the amount of the wetting agent(s) is up to 50 weight-%, such as from 5 to 40 weight-%, such as from 6 to 30 weight-% based upon all reactive components.

Moreover, a polymerizable mixture may contain one or more auxiliary components selected from, but not limited to, chelating agents, polymerization inhibitors, viscosity regulating agents, surface tension regulating agents, glass transition regulating agents, compatibilizing components, ultra-violet absorbing compounds, medicinal agents like ophthalmic pharmaceutical agents, ophthalmic demulcents, excipients, antimicrobial compounds, copolymerizable and non-polymerizable dyes, release agents, reactive tints, pigments, and chelating agents, and combinations thereof. In some embodiments, the sum of such auxiliary components may be up to 20 weight-%. Preferred embodiments may include photo initiators that create reactive species when exposed to one or more of: visible light, ultraviolet light, red light, and infrared light, and may include one or more of a: visible light, ultraviolet light, red light, and infrared light absorbing moiety,

A polymerizable mixture may be prepared, for example, by simple mixing of the constituents of the mixture. In some embodiments, reactive components (e.g., hydrophilic monomers, wetting agents, and/or other components) are mixed together with an inert diluent to form the polymerizable mixture. Such diluents may have the effect of controlling expansion of a prosthetic eye formed upon hydration, assisting in solubility of components, and regulating a glass transition temperature. Other embodiments may exclude the inert diluent.

Classes of suitable diluents include, without limitation, alcohols having 3 to 20 carbons, amides having 10 to 20 carbon atoms derived from primary amines, ethers, polyethers, ketones having 3 to 10 carbon atoms, and carboxylic acids having 8 to 20 carbon atoms. As the number of carbons increases, the number of polar moieties may also be increased to provide the desired level of water miscibility. In some embodiments, primary and tertiary alcohols are preferred. Preferred classes include alcohols having 4 to 20 carbons and carboxylic acids having 10 to 20 carbon atoms.

In some embodiments, the diluents are selected from 1,2-octanediol, t-amyl alcohol, 3-methyl-3-pentanol, decanoic acid, 3,7-dimethyl-3-octanol, 2-methyl-2-pentanol, 2-ethyl-1-butanol, 3,3-dimethyl-2-butanol, tripropylene methyl ether (TPME), butoxy ethyl acetate, mixtures thereof and the like.

In some embodiments, the diluents are selected from those that have some degree of solubility in water. In some embodiments at least about three percent of the diluent is miscible with water. Examples of water soluble diluents include, but are not limited to, 1-octanol, 1-pentanol, 1-hexanol, 2-hexanol, 2-octanol, 3-methyl-3-pentanol, 2-pentanol, t-amyl alcohol, tert-butanol, 2-butanol, 1-butanol, ethanol, decanoic acid, octanoic acid, dodecanoic acid, 1-ethoxy-2-propanol, 1-tert-butoxy-2-propanol, EH-5(commercially available from Ethox Chemicals), 2,3,6,7-tetrahydroxy-2,3,6,7-tetramethyl octane, 9-(1-methylethyl)-2,5,8,10,13,16-hexaoxaheptadecane, 3,5,7,9,11,13-hexamethoxy-1-tetradecanol, mixtures thereof and the like. Esters of alcohols, such as boric acid esters of alcohols, are other embodiments of diluents.

In some embodiments, an amount of diluent preferred is typically up to 60 weight-%, such as from 10 to 60 weight-%, such as from 20 to 50 weight-%, based upon the complete polymerizable mixture.

In another aspect, in some embodiments, a polymerizable mixture includes one or more cross-linkers in an amount of 0.5 to 5.0 weight-%, one or more non-reactive diluents (such as polyhydric alcohols, esters of polyhydric alcohols or ethers of polyhydric alcohols, e.g. glycerols and glycerol esters) in an amount of 0 to 60.0 weight-%, and one or more polymerization inhibitors in an amount of less than 100.0 ppm and preferably less than 50.0 ppm, based on the weight of the polymerizable mixture. A viscosity of the polymerizable mixture may also play an important role, and is typically 1-25 cP, such as 2-15 cP, in particular 3-10 cP, although other viscosities are within the scope of the present invention.

As mentioned above, the oxygen equilibrium concentration of the polymerizable mixture is preferably 0.05-8.0 volume-%, e.g., 0.2-6.0 volume-%, e.g., in the range of 0.5-6 volume-%. The lower limits (such as 0.05%, 0.1%, 0.2% etc.) are stated for practical reasons and it is quite possible to achieve even lower concentrations.

An Oxygen content of a polymerizable mixture may be adjusted to a desired level (X) by exposing the polymerizable mixture (previously being mixed under an ambient atmosphere (1013 mbar, 21 volume-% O2)) to a reduced pressure P, where P=X*1013/21 mbar. Subsequently, the reduced pressure (sometimes referred to as a “vacuum”) can be released and the oxygen-adjusted polymerizable mixture can be stored under an atmosphere having an oxygen concentration corresponding to a suitable atmosphere having an oxygen concentration of X.

In some preferred embodiments, the oxygen concentration in a controlled atmosphere ambient to the deposited polymerizable mixture, and a substrate in contact with the polymerizable mixture is lower than the oxygen equilibrium concentration in the polymerizable mixture.

### Three-Dimensional Printing Device

The deposition of a plurality of droplets is typically achieved by breaking a volume of Polymerizable Mixture into droplets and propelling the droplets in a desired direction at a predetermined velocity. In some embodiments, such generation of droplets of Polymerizable Mixture and propelling of the droplets in a given direction at a predetermined velocity may be accomplished via operation of an additive manufacturing printhead. Such printheads are capable of simultaneous deposition of a plurality of droplets of a liquid either in a one-dimensional pattern (e.g.; in the form of lines) or in a two-dimensional pattern. In some embodiments, a droplet is preferably in a smaller range for additive manufacturing, such as, for example, in a picolitre range, such as between about 3 picolitres and 20 picolitres per droplet and preferably 10 to 30 passes of a printhead in relation to a substrate. In some embodiments, a prosthetic eye is formed via 14 to 18 passes of a printhead expelling droplets in a desired pattern relative to a receiving surface.

In some embodiments, a desired speed and accuracy of a deposition of the plurality of droplets may be accomplished with an additive manufacturing printhead capable of simultaneous deposition of a two-dimensional pattern of polymerizable mixture so that a pattern (or multiple successive patterns) of droplets of the polymerizable mixture representing an integer map of digital value pattern (or a grayscale image) of a desired article (such as a prosthetic eye) can be printed.

In some preferred embodiments, dispersion and propulsion of droplets forming a two-dimensional pattern representing an integer map of digital value pattern in the form of a grayscale image is achievable in a single pass of a printhead depositing droplets in an area at least the size of the prosthetic eye. Commercially available printing heads suitable for this purpose include the Samba™ printhead from Fujifilm, e.g., Samba™ G3L Printhead which has 2048 nozzles per module and is capable of deposition of liquids in the order of 2.4 picolitre for native drop size to 13.2 picolitre maximum drop size at a 1200 native dpi accuracy.

A pattern of each pass of droplets expelled, propelled, and deposited by the 3D printing device, may be determined in relation to a desired transmissibility pattern of a prosthetic eye to be formed. A shape of the prosthetic eye being formed that correlates with the transmissibility pattern. For example (in case of a prosthetic eye), data gathered from measuring a patient's eye may be used to generate input. Data may include, for example, optical characteristics, surface properties, size and shape dimensions and observations of an ocular disease state.

Three-dimensional printable models may be created, by way of non-limiting example, based upon a computer aided design (CAD) software, or a scan of a patient's eye. Patient eye scanning may include collecting and analyzing digital data representative of a shape and appearance of the patient's eye. Based on collected data, a three-dimensional model of a target prosthetic eye may be produced. The 3D printable model may be processed by software to convert the model into a grayscale image (or other digital value patterns) and produce a file containing instructions tailored to a specific type of droplet expulsion devices, such as, for example, a 3D printer to repeatedly deposit polymerizable mixture according to the grayscale image or other digital value patterns.

### Substrate

The present invention provides for depositing a plurality of droplets of polymerizable mixture onto a surface of a substrate. Suitable materials for the substrate include one or more of: glass, polyolefins like polypropylene, polystyrene, and other smooth materials.

In some preferred embodiments, a form of a polymerizable mixture droplet receiving substrate represents a shape of one side of a resulting (non-hydrated) prosthetic eye, e.g., the substrate may include at least a portion that is arcuate or otherwise curved in a shape and size suitable for a prosthetic eye fitting a human eye socket. Similarly, a substrate may include a portion that is relatively flat in a size and shape suitable for a smaller prosthetic eye insertable into a smaller human eye socket.

A size of the substrate is preferably adjusted to fit a required dimension of a finished hydrated prosthetic eye. A substrate with a rotational axis may be formed by one or more of: lathing, grinding, injection molding, and additive manufacturing. A substrate that is not constrained to shapes with a rotational axis may be prepared, for example, via 3D printing. A substrate may therefore include an optical surface shape that is not spherical, such as a substrate surface shape duplicating, modeled after, or based upon a portion of a patient's eye exposed to air.

In some embodiments, in order to adjust wettability of a surface of a substrate that will receive polymerizable mixture, a surface of the substrate may be pre-treated with one or more of: a surfactant; exposed to UV; exposed to ozone; and exposed to plasma treatment; or a combination of such treatments. In some preferred embodiments, a receiving surface of a glass or polymer substrate may be pre-treated with Tween® 80 or a silicone surfactant such as Dow Corning™ Additive 67, Additive 14, Additive 57, Xiameter OFX-0193 etc. In some implementations of the present invention, a surfactant is included in the polymerizable mixture.

In some embodiments, a method of manufacture of a prosthetic eye includes bringing an oxygen concentration in the substrate into equilibrium with an oxygen concentration in the controlled atmosphere. Similarly, in some preferred embodiments, an oxygen concentration in the substrate is the same or less than an oxygen content of a polymerizable mixture deposited on the substrate.

In order to obtain an oxygen concentration in the substrate which is in equilibrium with the oxygen concentration in the controlled atmosphere, the substrate may be allowed to be exposed to the controlled atmosphere (or a corresponding atmosphere) prior to the deposition of the droplets, e.g., for a period of at least 8 hours.

In some embodiments, a substrate may only be capable of a limited amount of oxygen within it, hence, it may not be necessary to take any particular precautions with respect to the oxygen concentration in the substrate.

### Controlled Atmosphere

In some embodiments, an ambient atmosphere in which the deposition printing described herein takes place may be controlled. A controlled ambient atmosphere may include, by way of non-limiting example, one or both of: defined ranges of a specified gas, defined ranges of particulate and controlled wavelengths of light or other energy wavelengths. In some preferred embodiments, a suitably low concentration of oxygen is achieved in an atmosphere encompassing the polymerizable mixture such that an oxygen content of the polymerizable mixture is appropriately controlled. In some embodiments, the receiving surface of the substrate may be contained within the controlled ambient atmosphere.

By way of specific a non-limiting example, in some embodiments, a controlled atmosphere has an oxygen concentration of at the most 5.0 volume-%. In some embodiments, such as those including etafilcon A as a prosthetic eye material, an oxygen concentration in a controlled atmosphere is at most 2.0 volume-%, e.g., 0.01-2.0 volume-%, such as 0.03-1.5 volume-%, e.g., in the range of 0.05-1.2 volume-%, such as 0.1-1.1 volume-%, and more preferably at the most 1.0 volume-%. The lower limits (such as 0.01%, 0.03%, 0.05% etc.) are stated for practical reasons and it is quite possible to achieve even lower concentrations.

In another aspect, in some embodiments, a controlled atmosphere under which deposition of a polymerizable mixture takes place is most conveniently at a pressure of 1.0 atm. (1013 mbar) which corresponds to an oxygen concentration of 21 volume-%. A lower oxygen concentration than the 21 volume-% found in a normal atmosphere may suitably be obtained by mixing of atmospheric air with another gas, such as, in some preferred embodiments, an inert gas such as, one or more of: nitrogen, helium, argon, or other inert gas.

In some embodiments, a controlled atmosphere may include an inert gas, such as nitrogen, mixed in specific amounts with pure oxygen. One preferred approach for controlled atmosphere uses nitrogen as the inert gas to displace atmospheric oxygen and thereby achieve an oxygen concentration of a desired level.

An oxygen concentration may be monitored by an oxygen meter and regulated at one or more of: prior to an additive manufacturing process, at a start of an additive manufacturing procedure, and during the manufacturing procedure and preferably also intermittently or continuously controlled during a process preparing one or both of: an optical element and a substrate.

### Depositing Additive Droplets and Curing

The methods of the present invention include depositing successive passes of a plurality of droplets of a polymerizable mixture onto a receiving surface that may include one or more of: a substrate, an insert, and previously deposited droplets, under a controlled atmosphere. The droplets of polymerizable mixture are preferably emitted from a printhead and deposited based upon a two dimensional pattern representative of a digital value pattern or greyscale image, such as a grayscale image (or other light map representing light intensity). Following a deposition of droplets of the polymerizable mixture, the droplets may be exposed to controlled amounts of actinic radiation to cause a gelation process that pins the droplets of polymerizable mixture in place relative to the substrate.

A polymerizable mixture is typically deposited using a 3D printing device, such as, for example, the printing devices described herein. In embodiments, individual droplets have a volume of 0.5-50 pico liters (pL), such as 1-40 pL or 1.5-30 pL, and preferably 2.0-15 pL.

In some embodiments, each plurality of droplets of the polymerizable mixture is deposited onto a surface relative to a substrate. The surface relative to the substrate may include one or more of: an upper surface of the substrate; droplets previously deposited; and an article placed upon one or both of the substrate and the previously deposited droplets of polymerizable mixture. Polymerizable mixture that is deposited onto previously deposited polymerizable mixture may integrate into previously deposited polymerizable mixture such that a single mass of polymerizable mixture forms on the substrate with no discernable layers. The single mass of polymerizable mixture may include multiple optical interfaces and/or sub-optical elements formed by respective series of one or more steps of: deposition, integration, melding, gelling, and pinning of polymerizable mixture.

Droplets of polymerizable mixture deposited upon an article may attach to the article. The article may have a surface treated with a wetting agent. After coming into contact with the receiving surface, the polymerizable mixture may subsequently be exposed to limited actinic radiation or heat after the deposition of the final of the successive layers of droplets for forming the prosthetic eye.

In variations of the present invention where successive layers of deposited polymerizable mixture are exposed to actinic radiation (e.g., UV light), the polymerizable mixture may include a photo-initiator. In the variants of the present invention where the polymerizable material deposited in successive passes are exposed to heat, the polymerizable mixture may include a thermal initiator.

In some variants of the present invention, successive patterns of polymerizable mixture are exposed to intermittent radiation (e.g., UV light) after each deposition of a layer of droplets of the polymerizable mixture. A degree of polymerization obtained by such intermittent exposure to actinic radiation is typically the degree required for the purpose of pinning, or otherwise obtaining gelation of, the polymerizable mixture. Such obtaining of gelation may be used to control migration of deposited droplets from a first position on the receiving surface to another position on the receiving surface (and/or to control migration of the deposited polymerizable mixture off of the receiving surface).

In preferred embodiments, such control of migration allows for limited flow of deposited polymerizable mixture, but prevents unmitigated rearranging of the deposited polymerizable mixture. For example, in some embodiments, controlled migration allows gravity and other natural forces, such as surface tension and/or intermolecular forces to coalesce and smooth a surface of deposited polymerizable mixture. Deposited polymerizable mixture will also integrate deposited droplets with previously deposited polymerizable mixture, and meld into a smoothed surface of polymerizable mixture that may then be pinned in place with a gelation process.

Deposited polymerizable mixture is preferably exposed to pinning actinic radiation after each pass (or after a predetermined number of passes) of the printhead depositing the polymerizable mixture, and ultimately exposed to curing actinic radiation after a deposition of a final pass of a printhead depositing droplets polymerizable mixture to form the optical element.

A pass of the printhead may include one or both of: the printhead moving relative to a substrate onto which polymerizable mixture is received, a substrate and previously deposited polymerizable mixture) moving relative to a printhead; and/or a printhead and a substrate moving relative to each other as the polymerizable mixture is deposited.

In some preferred embodiments, at least a portion of a pattern of polymerizable mixture deposited via a pass subsequent to a first pass of the printhead, combines with, and integrates into, previously deposited polymerizable mixture, thereby forming a single volume of polymerizable mixture. Gravity induces a limited movement of the deposited polymerizable mixture resulting in a smoothing of the surface of the deposited polymerizable mixture. Movement of deposited polymerizable mixture based upon gravity may be limited by surface tension forces and micro forces. Preferably the single volume of polymerizable mixture is pinned and/or gelled by exposure to a limited amount of actinic radiation thereby limiting additional movement following the smoothing effect of gravity induced movement.

In some variants of the present invention, a series of successive passes of a printhead, such as, for example, between two to twenty (2-20) passes of polymerizable mixture are deposited before exposing the deposited polymerizable mixture to intermittent actinic radiation that is effective to pin and/or gel the deposited polymerizable mixture. Prior to exposure to the intermittent actinic radiation, the deposited polymerizable mixture may undergo limited migration as a single volume of polymerizable mixture.

In some embodiments, a thickness of polymerizable mixture deposited in a pass of the printhead relative to a receiving surface, on any particular portion of the receiving surface, may be up to 50 μm (fifty micrometers), but preferably a maximum of 25 μm (twenty-five micrometers). Some embodiments may include up to 10 μm of polymerizable mixture deposited in a single pass of the printhead relative to the receiving surface (which may include one or more of: a substrate, previously deposited polymerizable mixture, an insert, and a formed prosthetic eye).

In some variants of the present invention, a polymerizable mixture includes a plurality of photo-initiators, with two or more photoinitiators having responsiveness to different wavelengths of actinic radiation. This is particularly interesting when it is desirable to utilize UV light at one wavelength for the intermittent exposure (for pinning or gelation) and another wavelength for final curing of deposited polymerizable mixture to form the optical element. Accordingly, in some variants of the present invention, a first polymerization initiator is used (in conjunction with exposure to an appropriate first actinic radiation) to create a construct of partially polymerized polymerizable mixture, and a second polymerization initiator is used (in conjunction with exposure to an appropriate second actinic radiation) to complete a curing process.

In addition to controlling a level of oxygen in the polymerizable mixture to within a desired range of oxygen content, some embodiments of the present invention include controlling polymerization of the deposited polymerizable mixture such that an optical element is being fabricated using the processes described in this disclosure, a degree of polymerization of deposited polymerizable mixture within a specified timeframe following deposition is limited to a degree of gelation to stop, or substantially slow, a movement of the polymerization mixture while allowing droplets from subsequent passes to meld into previously deposited polymerizable mixture and form a structure for an optical element with limited distortion.

A process for intermittent gelation may sometimes be referred to as pinning or gelling of the deposited polymerizable mixture. In some variants of the present invention, a pinning process may include applying a dose of actinic radiation in an intensity, wavelength, and length of time suitable to cause gelation, such as, for example, application of ultraviolet (UV) light to a UV curable polymerizable mixture and/or ink (UV ink). Actinic radiation wavelengths may be matched to photochemical properties of a polymerizable mixture and/or UV ink used in a manufacturing process. The actinic radiation may be any radiation that produces a desired photochemical activity, which in this case is polymerization of the polymerizable material.

As a result of the intermittent gelation, deposited polymerizable mixture and/or ink droplets move to a higher viscosity state, but stop short of full cure. Variants of the present invention that include pinning or gelation (or gelling) have enhanced ability to manage a flow and form of deposited polymerizable mixture, which in turn provides high optical qualities in an optical element formed via final cure of the deposited polymerizable mixture. For example, sufficient flow to allow gravity and/or other natural forces, such as, without limitation: surface tension and microforces to smooth a surface of the gelled polymerizable mixture but not significantly change a shape of an optical element is preferred. In some variants, other forces, such as, for example, centrifugal force may be used to form a surface shape.

Processes for gelation and cure may be modified based upon a selection and/or a concentration of one or more of: photo-initiators, cross-linkers, source of actinic radiation (e.g., UV light source), intensity of actinic radiation and duration of exposure. Examples of sources of actinic radiation may include light emitting diodes (LED) or light bulbs, lasers, and other emitters.

In some specific embodiments, a polymerizable mixture may include two photo-initiators absorbing energy at two different wavelengths. The polymerizable mixture is used with corresponding UV LED light sources (e.g., at 365 nm and at 400 nm). One initiator may be present in a concentration capable of starting gelation of the polymerizable mixture but insufficient to complete the polymerization. This enables individual deposits of polymerizable mixture to come to a same (or similar) relative degree of conversion prior to final cure. A final polymerization throughout an article formed by multiple subsequent depositions and pinning of polymerizable mixture (e.g.; an optical element) may be done as a separate step using another photo-initiator/UV LED light combination (e.g.; second photoinitiator's actinic radiation energy, or third photoinitiator's etc.) actinic radiation energy resulting in a uniform polymer network required for optical function.

As an alternative hereto, a thermal initiator that is active at or above a Tg may be used instead of, or in addition to, the second (or other) photo-initiator to complete curing of the deposited polymerizable mixture. The present invention also provides that without control of an oxygen content of deposited polymerizable mixture throughout deposition process steps, and during a final curing step, the oxygen inhibition effects would adversely impact the uniformity of the polymer network and may lead to creation of incompletely cured polymer and therefore a device with a tacky surface.

In some embodiments, a polymerizable mixture is deposited onto a curved surface, a first deposition, or multiple depositions, may be deposited as patterns of droplets of polymerizable mixture onto a curved surface, according to the methods described herein. The patterns of droplets include a volume and distribution that permits surface tension to maintain the patterns of droplets of polymerizable mixture with limited flow or other movement until partially cured with a gelation process, thereby pinning the deposited polymerizable mixture in place (and limiting flow).

Subsequent deposition of additional droplets from the printhead may fill in spaces left by a first deposition or subsequent layers until a surface of a receiving substrate is completely covered with polymerizable mixture and the deposited polymerizable mixture establishes a foundation to build an optical element upon. Alternatives to dot patterns include deposition of droplets to form a very thin layer (e.g.; 1 micron to 8 microns) and building an optical element upon such very thin layer with the processes disclosed herein.

In some embodiments, it is useful to isolate a printhead containing monomer from receiving actinic radiation, such as UV radiation in order to prevent premature gelation or polymerization of monomers in a printhead which may render the printhead inoperable, or operable at diminished performance levels. Isolation from actinic radiation is particularly important when using reactive monomers with low levels of inhibitors and/or using a printhead in an environment with low oxygen levels. Isolation may be accomplished, for example, via physical separation of the printhead and actinic radiation.

In order to achieve concurrent printing and pinning of a material to stop or slow the movement of deposited ink or polymerizable mixture once deposited, some embodiments of the present invention include apparatus operative to isolate a source of actinic radiation (e.g., UV light source) from the printhead to essentially eliminate or substantially reduce a potential of gelation/polymerization of polymerizable mixture in the printhead. Isolating the printhead from a source of actinic radiation and controlling both oxygen levels and movement of polymerization mixture, enables the fabrication of precise shapes and optical devices without artifacts in the matrix that adversely affect an optical performance of a final prosthetic eye that is fabricated.

In some embodiments, it may be preferred to wash gelled deposited polymerizable mixture with a solvent or water, e.g., to remove excess monomers, after multiple depositions of polymerizable mixture(s) and gelation processes have been completed, but before a final curing process is performed.

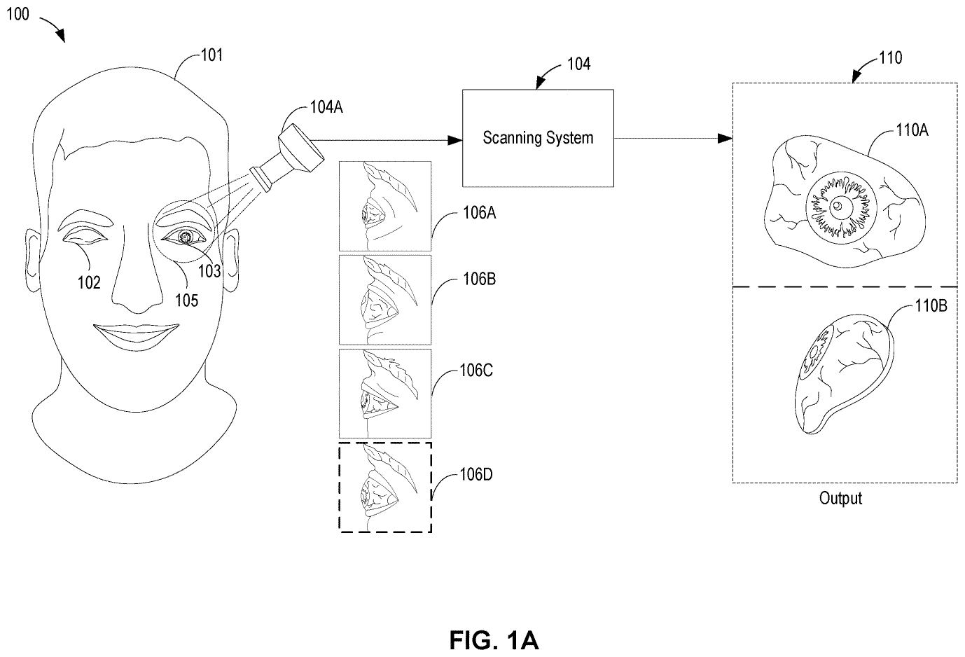

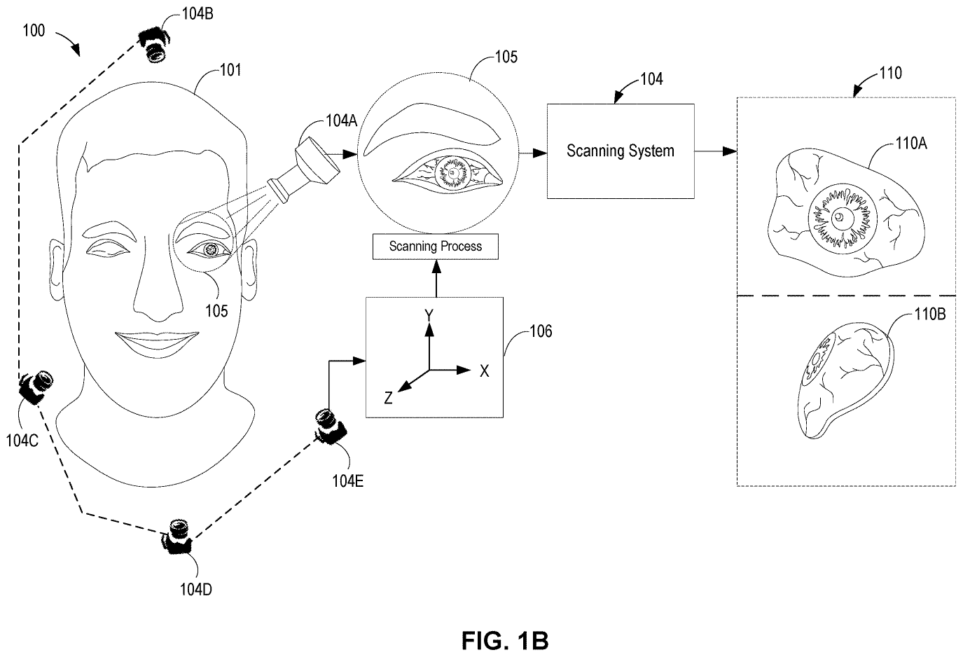

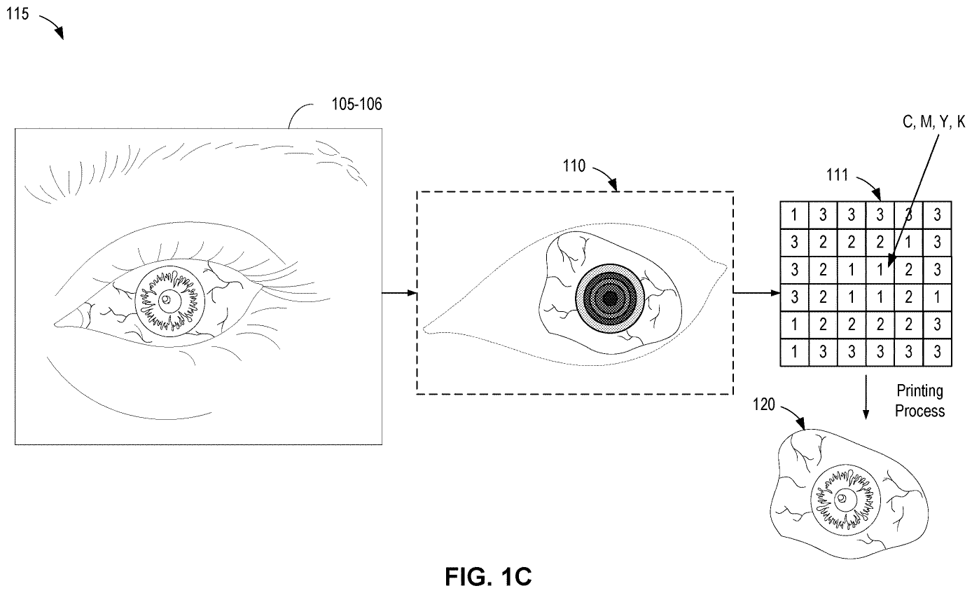

Referring to FIGS. 1A-1B, an exemplary scanning process 100 is illustrated for scanning a functional eye 103 of a user 101 to create a prosthetic eye model 110, according to some embodiments of the present invention. The user 101 may have a damaged eye 102 and a functional eye 103. In cases where the user 101 requires a prosthetic eye for the damaged eye 102, it is desirable for the prosthetic eye to resemble the functional eye 103 as closely as possible. This resemblance is important for both aesthetic appeal and psychological benefits, as the natural appearance of the prosthetic eye enhances the user's self-confidence and facilitates social interactions. The resemblance may also help maintain visual symmetry, which is significant for applications such as facial recognition and other visual-based systems.

The scanning process comprises a scanning system 104, which may include a first scanning device 104A and one or more other scanning cameras 104B-104E. The scanning system 104 may be equipped with various hardware components, including high-resolution optical sensors, image processors, and integrated light sources, as well as software systems for data analysis and model generation. The scanning system 104 may operate using advanced imaging algorithms, artificial intelligence, and machine learning techniques to accurately capture and interpret the physical and anatomical features of the functional eye 103. In some embodiments, the first scanning device 104A may alone be used to scan the functional eye 103, while in other embodiments, the additional scanning cameras 104B-104E may supplement the scanning process by capturing details from multiple angles.

The first scanning device 104A may be used to scan a front view 105 of the functional eye 103 to capture various anatomical and optical details. These details may include the shape, size, and color of the iris, the pupil's dimensions and location, the distribution of scleral veins, and the overall curvature of the eyeball. The first scanning device 104A may also capture the contours and patterns within the iris, which contribute to the eye's unique appearance. Advanced imaging technologies, such as optical coherence tomography (OCT) or high-resolution RGB cameras, may be used in the first scanning device 104A to achieve accurate and detailed image capture. The device may also incorporate depth sensors to assess the three-dimensional structure of the functional eye 103.

In some embodiments, the one or more other scanning cameras 104B-104E may be strategically positioned around the user 101 to scan the functional eye 103 from different angles and directions (106). These additional scanning cameras 104B-104E may capture details such as relative XYZ coordinates of various anatomical parts of the eye, including the sclera, eyelid contours, and peripheral features of the eyeball. The scanning cameras 104B-104E may generate side views and angular contours 106A-106D of the functional eye 103. These cameras may utilize structured light scanning, stereo imaging, or LiDAR technologies to capture precise measurements and create a comprehensive three-dimensional representation of the functional eye 103.

The working of the one or more other scanning cameras 104B-104E may involve capturing multiple images or data points from various angles around the functional eye 103. These data points may then be combined using image stitching algorithms to create a complete three-dimensional model. For example, scanning camera 104B may capture the upper-left quadrant of the functional eye 103, while scanning camera 104C may focus on the lower-left quadrant. Similarly, scanning cameras 104D and 104E may capture the corresponding right-side quadrants. The data from all cameras may be processed to form a unified representation of the functional eye 103, which includes both surface and subsurface details.

The scanning system 104 may then process the captured data to generate a prosthetic eye model 110 of the functional eye 103. The prosthetic eye model 110 may include a detailed front model view 110A and a side model view 110B. The front model view 110A may replicate the appearance of the iris, pupil, and scleral patterns, while the side model view 110B may include the contours, curvature, and overall geometry of the eye. The prosthetic eye model 110 serves as the digital blueprint for fabricating the prosthetic eye, which is specifically designed to replace the damaged eye 102 of the user 101.

The prosthetic eye model 110 may further be used in an additive manufacturing system to print the prosthetic eye with high precision. The additive manufacturing process may involve layer-by-layer deposition of biocompatible materials using patterned droplets, guided by the digital design of the prosthetic eye model 110. For example, the front model view 110A may guide the printing of the iris and pupil details, while the side model view 110B may dictate the overall curvature and structure of the prosthetic eye. This approach facilitates that the prosthetic eye accurately replicates the functional eye 103 in terms of both appearance and fit.

In embodiments where the first scanning device 104A is used alone, the scanning system 104 may incorporate advanced imaging capabilities to capture all necessary details from a single perspective. This may involve using multi-spectral imaging to capture different wavelengths of light, thereby revealing details that are not visible in standard RGB imaging. For example, near-infrared imaging may be used to highlight blood vessel patterns, while ultraviolet imaging may reveal surface irregularities.

In embodiments involving multiple scanning cameras 104B-104E, the scanning system 104 may employ synchronization mechanisms to coordinate data capture across all cameras. This synchronization may include time-stamped imaging or simultaneous triggering of all cameras to avoid discrepancies in the captured data. The processed data may then undergo computational analysis to correct any distortions or misalignments, resulting in a high-fidelity model of the functional eye 103.

The scanning system 104 may also include software modules for enhancing the captured images and data. These modules may apply noise reduction, edge detection, and pattern recognition algorithms to refine the details of the functional eye 103. For example, the software may enhance the contrast of the iris patterns or sharpen the edges of the pupil to improve the accuracy of the prosthetic eye model 110.

The prosthetic eye model 110 generated by the scanning system 104 may also include metadata, such as the colorimetric properties of the iris or the reflectivity of the sclera. This metadata may be used to select appropriate materials and pigments during the additive manufacturing process. For example, the colorimetric data of the iris may guide the selection of CMYK pigments for printing the prosthetic eye.

In some embodiments, the scanning system 104 may incorporate real-time feedback mechanisms to validate the captured data. For example, the system may compare the scanned data with pre-defined templates or reference models to identify any missing details or inconsistencies. This feedback may enable immediate adjustments to the scanning process, facilitating that all necessary details are captured before generating the prosthetic eye model 110.

The prosthetic eye model 110 may also include variations to account for individual user preferences or functional requirements. For example, the model may include a slightly larger scleral region to improve the fit within the ocular socket, or additional surface texturing to enhance comfort. These variations may be incorporated into the model during the data processing phase.

Referring now to FIG. 1C, an exemplary process 115 is illustrated for generating a digital value pattern 111 and an energy intensity pattern 120 from the scanned eye data, which may include both a front view 105 and views from different angles and directions 106 of a functional eye, for guiding the printing processes described later in various figures, according to some embodiments of the present invention. The scanned eye data 105-106, as previously described in FIGS. 1A-1B, provides comprehensive information about the anatomical and optical details of the functional eye (103), such as the iris patterns, pupil dimensions, scleral veins, and contours. This data may be used to generate a prosthetic eye model 110, which acts as a digital representation of the functional eye. The prosthetic eye model 110 may then be processed further to create a digital value pattern 111 and an energy intensity pattern 120, which may individually or collectively guide the printing of the prosthetic eye for the damaged eye (102) of the user (101).

The digital value pattern 111 may be generated by converting the prosthetic eye model 110 into a grid-like structure, where each grid value corresponds to a specific area of the prosthetic eye. Each value in the digital value pattern 111 may represent the amount and type of polymerizable mixture that needs to be emitted by a print head for that area during the printing process. For example, values in the digital value pattern 111 corresponding to the iris region of the prosthetic eye model 110 may encode information about the colors, textures, and material composition required for accurately replicating the intricate iris patterns of the functional eye. For example, a digital value of “3” may represent a high concentration of cyan pigment for a specific radial pattern in the iris, while a value of “1” may correspond to a low concentration of yellow pigment for a subtle shading effect.

In some embodiments, each value within the digital value pattern 111 may have an associated marking corresponding to one or more CMYK (Cyan, Magenta, Yellow, and Black) pigments, denoting which pigments are to be deposited at a specific location during the additive manufacturing process. The numerical values 1, 2, 3 within the digital value pattern represent the quantity or intensity of the corresponding pigment(s) to be emitted by the print heads. For example, a value of 1 may indicate a minimal deposition of a particular pigment, a value of 2 may indicate a moderate deposition, and a value of 3 may signify a higher concentration or saturation of the selected pigment. This structured deposition approach allows for precise control over the pigmentation process, enabling the formation of complex patterns such as vein structures within the sclera, radial gradients in the iris, or depth-enhanced pupil regions, thereby enhancing the overall realism of the prosthetic eye.

In embodiments where the energy intensity pattern 120 may be utilized, this pattern represents the optical and material properties of the prosthetic eye in a way that correlates with the intensity of energy required to polymerize or deposit the material in specific regions. For example, the energy intensity pattern 120 may dictate that the central region corresponding to the pupil is printed with an opaque polymerizable mixture that absorbs less energy, whereas the surrounding iris area is printed with semi-transparent or pigmented materials requiring higher energy levels for polymerization. This pattern enables precise control over the activation of print heads and the emission of the required polymerizable mixtures.

One or both of the digital value pattern 111 and the energy intensity pattern 120 may collectively be referred to as the prosthetic model or prosthetic eye model, which serves as the foundational input for the additive manufacturing processes that print the prosthetic eye. During the printing process, the prosthetic model guides the print heads to emit the appropriate polymerizable mixtures in specific quantities and types. For example, the digital value pattern 111 may trigger a print head equipped with ophthalmic-grade CMYK pigments to deposit small droplets of a cyan polymerizable mixture at precise locations, forming the radial patterns of the iris. Similarly, the energy intensity pattern 120 may instruct another print head to deposit transparent material for creating a glossy layer that mimics the natural corneal surface.

In some embodiments, the prosthetic model may be dynamically updated during the printing process based on real-time feedback from sensors or imaging systems integrated into the printing apparatus. For example, if the printed layers deviate from the prosthetic eye model 110 due to environmental factors such as temperature or material flow rate, the digital value pattern 111 and energy intensity pattern 120 may be recalibrated to correct the deposition parameters. This adaptability facilitates that the final prosthetic eye closely replicates the appearance and structure of the functional eye.

The prosthetic eye model 110, as represented by the prosthetic model, facilitates advanced customization options for the prosthetic eye. For example, users may choose to modify the color intensity of the iris or add aesthetic features such as starburst patterns. These customizations are encoded into the digital value pattern 111, which adjusts the material deposition accordingly during printing. Similarly, the energy intensity pattern 120 may be adapted to create specialized effects, such as a matte scleral surface or a reflective iris.

The prosthetic model also allows for precise replication of anatomical features that contribute to the natural appearance of the prosthetic eye. For example, the digital value pattern 111 may include data for printing fine scleral veins using red and blue pigments, with each vein's thickness and branching pattern derived from the scanned eye data 105-106. The energy intensity pattern 120 may regulate the polymerization process to achieve the desired translucency and depth for these veins, enhancing the realism of the prosthetic eye.

In another embodiment, the prosthetic model can be utilized to incorporate functional enhancements into the prosthetic eye. For example, regions of the digital value pattern 111 corresponding to the pupil may include instructions for embedding a dynamic pupil mechanism, while the energy intensity pattern 120 may guide the integration of electronic sensors or micro-cameras into specific layers of the prosthetic eye. These enhancements can provide additional functionality, such as simulating pupil dilation or capturing visual data for external processing.

The energy intensity pattern 120 can also optimize the printing process by reducing material waste and improving efficiency. For example, areas of the prosthetic eye model 110 that require minimal material deposition, such as thin scleral regions, may have lower energy values in the pattern 120, reducing the activation of print heads in those areas. Conversely, regions with complex features, such as the iris, may have higher energy values to provide precise layering and color blending.

In addition to guiding the printing process, the prosthetic model may serve as a quality control reference for the printed prosthetic eye. After printing, the prosthetic eye can be scanned and compared against the digital value pattern 111 and/or energy intensity pattern 120 to identify any discrepancies. If deviations are detected, the prosthetic model can be used to adjust the printing parameters for subsequent iterations, providing consistency across multiple prosthetic eyes.

The energy intensity pattern 120 may also support advanced printing techniques, such as multi-material deposition or gradient layering. For example, the pattern may instruct the print heads to transition from a transparent polymerizable mixture to a pigmented mixture within the same layer, creating seamless gradients that mimic the natural color variations of the iris. This capability enhances the aesthetic appeal and realism of the prosthetic eye.

The prosthetic model, comprising the digital value pattern 111 and energy intensity pattern 120, is a versatile and comprehensive representation of the prosthetic eye model 110. By integrating detailed anatomical data and advanced material properties, the prosthetic model enables the creation of highly realistic and functional prosthetic eyes through additive manufacturing. This process not only replicates the natural appearance of the functional eye but also allows for the incorporation of innovative features and customizations, addressing the diverse needs of users.

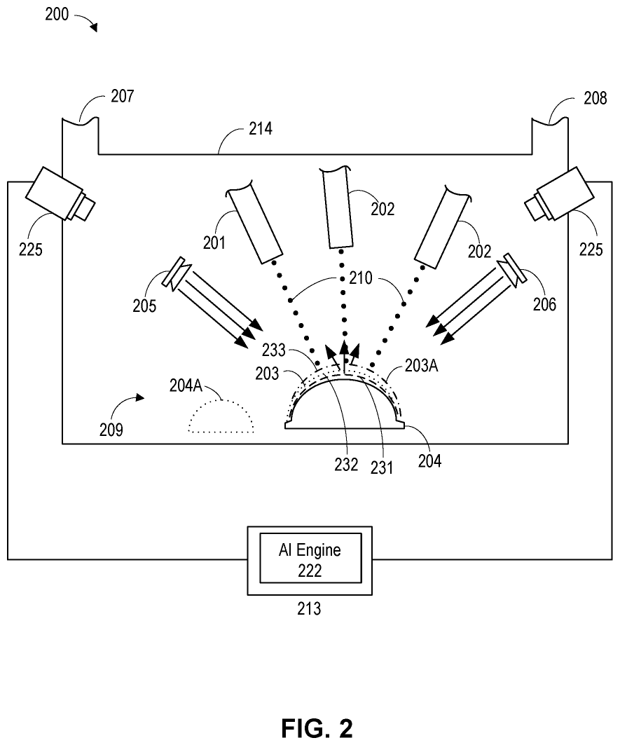

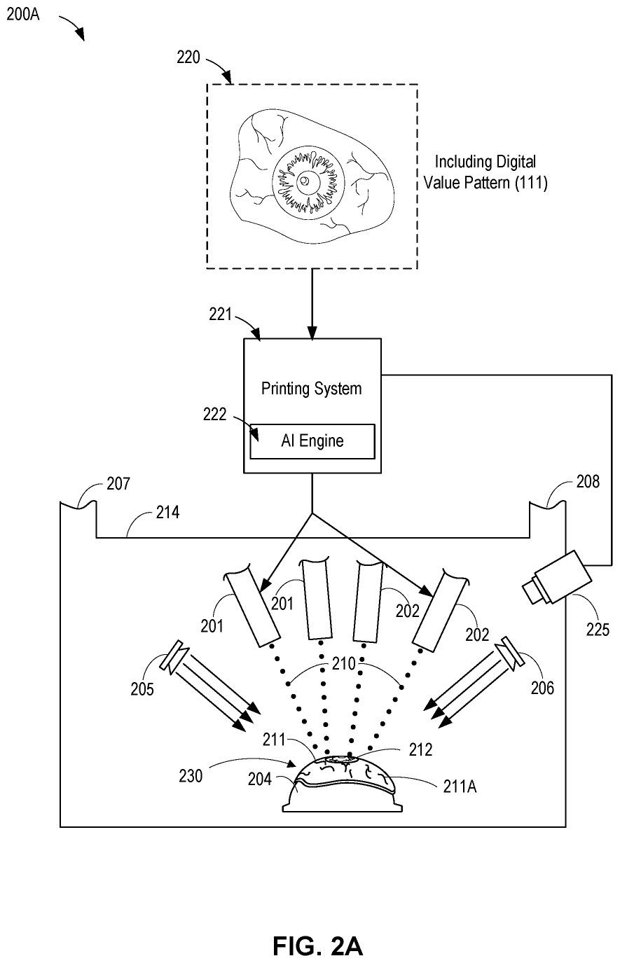

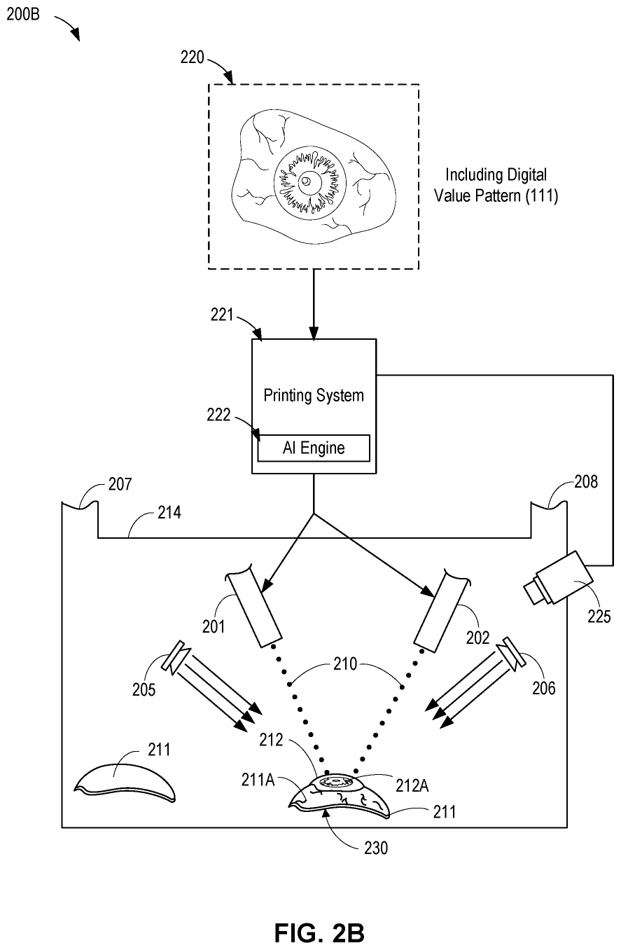

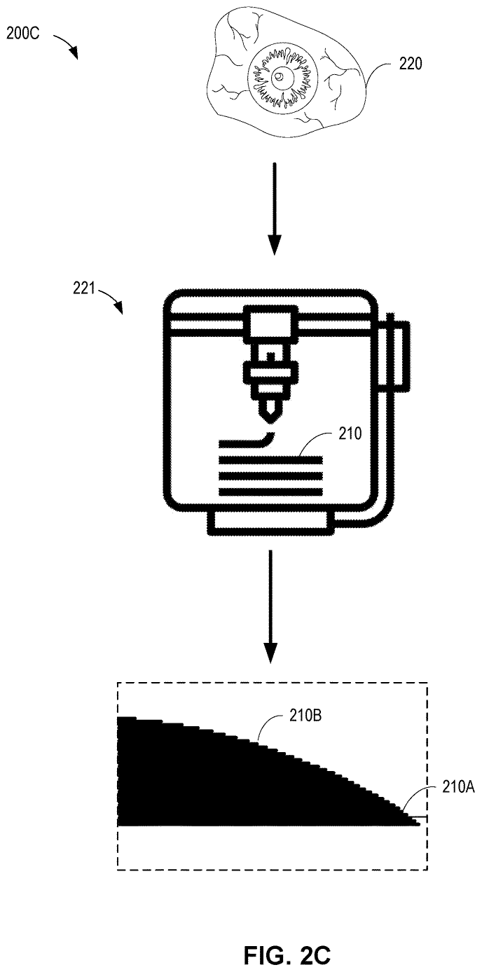

Referring now to FIG. 2, an exemplary additive manufacturing system 200 is illustrated for the additive manufacturing of a prosthetic eye, employing multiple print devices 201 and 202 (print heads), controlled by an AI engine 222, to deposit polymerizable mixtures 203 in a layer-by-layer fashion using patterned droplets. This process begins with the deposition of droplets 210 of the polymerizable mixture onto a receiving surface 203A, supported by a substrate 204. The substrate 204, which may include a smooth and arcuate receiving portion 204A, is designed to act as the base for building the prosthetic eye, offering structural support for the deposited layers. The additive manufacturing process uses a series of precisely calibrated steps, incorporating real-time adjustments facilitated by the AI engine 222 to achieve accuracy in replicating the prosthetic eye's complex anatomical features.

The print devices 201 and 202 are configured to emit droplets 210 in predefined patterns, as dictated by a prosthetic model (e.g., 111 and/or 120). Each droplet represents a specific volume of the polymerizable mixture 203, deposited to form layers such as the first layer 231, second layer 232, and third layer 233. These layers may include structural and optical components such as the base, iris, pupil, and scleral details of the prosthetic eye. For example, the first layer 231 may constitute the prosthetic base, while subsequent layers, such as 232 and 233, may include detailed patterns resembling the natural iris and sclera. Actinic radiation sources 205 and 206 are strategically positioned to provide intermittent curing, partially polymerizing each layer before the next layer is deposited. This pinning process stabilizes the material, maintaining the desired dimensions and alignment throughout the additive manufacturing process.

The AI engine 222, interfaced with the controller 213, orchestrates the operation of the print devices 201-202 and the actinic radiation sources 205-206. The AI engine 222 processes input data, including the energy intensity or digital value map or pattern of the prosthetic eye and real-time feedback from cameras 225. These cameras may be positioned to capture the deposition process and relay information about droplet placement, size, and alignment. Based on this data, the AI engine 222 dynamically adjusts the printhead trajectory, droplet emission rates, and curing intensity to correct any deviations from the digital design. For example, if an area of the second layer 232 exhibits uneven material distribution, the AI engine 222 can modify the deposition path or adjust the volume of polymerizable mixture 203 emitted by the print devices.

The controlled atmosphere 209 within the enclosure 214 enhances the precision and quality of the manufacturing process. Ports 207 and 208 allow for the introduction of gases such as nitrogen to regulate oxygen levels, reducing the risk of premature polymerization and providing consistent material properties. This atmosphere may also support the curing process by maintaining optimal environmental conditions for the actinic radiation sources 205-206 to function effectively. For example, the actinic radiation sources can emit UV light at specific wavelengths to cure different materials used in the prosthetic eye, such as transparent layers or pigmented regions.

The substrate 204, which supports the receiving surface 203A, may be transparent to actinic radiation, enabling the curing process to occur from both above and below the substrate. This flexibility allows for intricate patterns, such as iris details or vein-like structures in the sclera, to be cured uniformly without distortion. In some embodiments, the print devices 201-202 may work in tandem with alternate actinic radiation configurations, such as angled or lateral sources, to cure specific regions selectively, enhancing the precision of complex features.

The printing process incorporates a digital optical design encoded with information about the energy intensity pattern and digital value pattern of the prosthetic eye. One or both patterns may guide the additive manufacturing system 200 in determining the type, volume, and location of polymerizable mixtures to be deposited. For example, a grayscale energy intensity pattern may dictate that the central pupil region is printed with an opaque black polymerizable mixture, while the surrounding iris region transitions seamlessly to semi-transparent or pigmented layers. Similarly, digital values corresponding to the scleral region may include instructions for embedding fine veins using red and blue pigments, replicating the natural appearance of a human eye.

The cameras 225, integrated into the additive manufacturing system 200, continuously monitor the formation of the prosthetic eye, capturing high-resolution images of each layer. This data is analyzed by the AI engine 222 to verify alignment with the digital design. If the cameras detect a misalignment in the third layer 233, for example, the AI engine 222 can compensate by adjusting the subsequent deposition of material in the fourth or fifth layers. This real-time feedback loop facilitates that the prosthetic eye retains its intended structural and aesthetic properties throughout the manufacturing process.

The actinic radiation sources 205-206 play a vital role in the final curing stages of the prosthetic eye. After the final layer is deposited, the prosthetic eye undergoes a complete curing process, solidifying all layers and enhancing the durability and stability of the final product. This curing step may involve multiple cycles of actinic radiation at varying wavelengths, tailored to the specific materials used in the prosthetic eye. For example, UV-A radiation may be applied to cure transparent corneal layers, while UV-B radiation targets pigmented regions to enhance color retention and stability.

The AI engine 222 also facilitates advanced customization of the prosthetic eye. Based on input from diagnostic tools, such as measurements of the user's functional eye or preferences for iris patterns and colors, the AI engine 222 adjusts the deposition process to produce a prosthetic eye tailored to the user's requirements. For example, if the user desires a blue iris with subtle green accents, the print devices 201-202 can emit corresponding polymerizable mixtures in precisely controlled volumes to achieve the desired aesthetic effect.

After the additive manufacturing process is complete, the prosthetic eye undergoes a hydration step, where it is immersed in a biocompatible solution. This step allows the eye to achieve its final dimensions and flexibility, enhancing its fit and comfort for the user. The hydrated prosthetic eye is then polished to achieve a smooth, glossy finish, replicating the natural sheen of a human eye.

In some embodiments, each of the print heads 101-102 may be individually utilized to deposit a specific C, M, Y, K pigment based on the colors, sizes, and patterns defined by the prosthetic eye model and the values within the digital value pattern 111. The deposition process is dynamically controlled to achieve a realistic pigmentation effect, so that the appropriate pigments are deposited in precise locations to form the sclera, vein patterns, iris, and pupil. The timing of the deposition of each C, M, Y, K pigment may depend on the prosthetic eye model, such that different pigments are emitted at different stages of the additive manufacturing process to allow proper layering and blending. For example, a cyan and magenta combination may be deposited first to create a vein pattern with a bluish-red appearance, while yellow and black pigments may be deposited later to define depth variations within the iris and pupil. This controlled timing mechanism enhances the overall visual realism of the prosthetic eye, allowing for the formation of intricate patterns and subtle color transitions that closely replicate the appearance of a natural human eye.

Referring now to FIG. 2A, an exemplary printing process 200A is illustrated for manufacturing a prosthetic eye 230 on a substrate 204, including the printing of a prosthetic base 211 and the iris 212, according to some embodiments of the present invention. The process begins with a prosthetic model 220, which may be generated based on the scanned data of a functional eye, as previously described. The prosthetic model 220 is fed into a printing system 221, which may comprise an AI engine 222, controllers for coordinating the print heads 201-202, and actinic radiation sources 205-206 for curing the deposited polymerizable mixtures 210. The printing system 221 manages the entire printing process in real-time, so that the prosthetic eye 230 is constructed with high precision and accuracy.

The printing process first focuses on constructing the prosthetic base 211 layer by layer using deposited patterns of droplets on the substrate 204. The substrate 204, which may include a smooth and arcuate receiving surface (204A), serves as the foundation for the deposited polymerizable mixtures 210. The substrate 204 may be pre-fabricated from materials such as polymethyl methacrylate (PMMA) or silicone, offering stability and biocompatibility. The prosthetic base 211 may incorporate the scleral portion of the prosthetic eye, including the veins 211A, which are printed using the same or additional print heads under the guidance of the prosthetic model 220. Each layer of the prosthetic base 211 is formed by the controlled deposition of polymerizable mixtures 210, which may include a combination of opaque white inks for the scleral portion and pigmented inks for the vein patterns.

The polymerizable mixture used for printing the prosthetic base 211 may be tailored to meet the requirements of durability, biocompatibility, and visual realism. For example, the scleral portion may be printed using an opaque white polymerizable mixture that provides a natural-looking base color. Veins 211A are printed using red and blue pigmented inks, which are deposited in fine patterns to replicate the intricate vascular structure of a natural eye. The droplet size and placement are precisely controlled by the print heads 201-202, which are managed by the AI engine 222 based on the prosthetic model 220. After each layer is deposited, the actinic radiation sources 205-206 partially cure the material, stabilizing the structure and preparing it for the deposition of subsequent layers.

Once the major portion of the prosthetic base 211 is completed, the printing process transitions to the iris 212, including the pupil pattern. The iris 212 may be printed directly onto the prosthetic base 211 using one or more print heads 201-202, operating in a synchronized manner. The iris 212 may be constructed layer by layer (by depositing droplets), with each layer contributing to the intricate radial and concentric patterns that define the appearance of a natural iris. The pupil pattern is printed at the center of the iris 212 using an opaque black ink, providing a realistic representation of the functional eye's pupil. The polymerizable mixtures used for the iris 212 may include ophthalmic-grade CMYK pigments, which are capable of producing vibrant and lifelike colors. These pigments are deposited in precise volumes and patterns, as dictated by the prosthetic model 220.

The printing of the iris 212 is further enhanced by the use of transparent polymer layers, which are intermittently deposited between the pigmented layers. These transparent layers add depth and texture to the iris 212, creating a three-dimensional effect that closely resembles the natural structure of a human iris. The transparent layers also protect the pigmented layers from wear and provide a glossy finish that enhances the aesthetic appeal of the prosthetic eye 230. The AI engine 222 continuously monitors the printing process, analyzing real-time data from cameras 225 to verify the alignment and accuracy of the deposited layers.

In some embodiments, each of the individual print heads 201-202 (which may be more than one for each color pigmentation) may be utilized to deposit droplets 110 of different CMYK pigmentation, as well as a transparent polymerizable mixture, to form various components of the prosthetic eye, including the sclera, iris, pupil, and vein patterns. The deposition of these polymerizable mixtures is precisely controlled based on the prosthetic eye model 220 to achieve the intended color, shape, size, and pattern of each component. The sclera may require a predominantly white base with subtle red or blue veins, which is achieved by selectively depositing a low concentration of cyan and magenta (C+M) pigments from corresponding print heads 201-202 in thin, branching vein-like patterns while maintaining a mostly transparent or white base. The iris is formed using different combinations of CMYK pigments, where green and blue eye colors may be achieved by varying ratios of cyan and yellow (C+Y), whereas brown or hazel eye colors may be produced by increasing magenta and yellow (M+Y) pigments. The pupil is typically black, which is formed by depositing a high density of black (K) pigment in a circular pattern at the center of the iris. Additionally, a transparent polymerizable mixture may be intermittently deposited between layers to enhance the depth effect and replicate the natural refractive properties of a human eye.

The amount of each pigment to be deposited is determined based on the digital value pattern 111 (FIG. 1C), which dictates the intensity and layering of each CMYK pigment at specific locations of the prosthetic eye. The digital value pattern 111 assigns numerical values (e.g., 1, 2, 3) to define the concentration and frequency of deposition, where a value “1” may indicate low pigment deposition, a value “2” may indicate moderate deposition, and a value “3” may indicate high pigment density at a given coordinate. For example, in areas of the sclera where veins are required, a value of “1” for cyan and magenta (C+M) may be assigned to generate faint capillary structures, while a value of “3” in the pupil region ensures a dense black (K) deposition to create a dark, well-defined pupil. Similarly, in the iris, the deposition of CMYK pigments is dynamically adjusted based on the selected eye color, allowing for smooth color gradients and fine variations in pigmentation. The print heads 101-102 are controlled to dispense these droplets layer-by-layer, facilitating seamless integration of pigments and achieving an ultra-realistic prosthetic eye that closely resembles a natural human eye.

The cameras 225, which are strategically positioned around the printing area, capture high-resolution images and video feeds of the additive manufacturing process. These cameras are coupled to the printing system 221 and provide real-time feedback to the AI engine 222. If any discrepancies are detected, such as misalignment of the printed layers or uneven material deposition, the AI engine 222 adjusts the movement of the print heads 201-202 or modifies the deposition parameters to correct the issue. For example, if the cameras 225 detect that the vein patterns 211A on the scleral portion are not aligning with the intended design, the AI engine 222 can recalibrate the print heads to reposition the droplets 210 accurately.

The final step in the printing process involves the application of a protective top layer over the iris 212 and the prosthetic base 211. This layer may be printed using a transparent polymerizable mixture that enhances the durability and longevity of the prosthetic eye 230. The protective layer also provides a natural sheen, mimicking the glossy appearance of a human eye's cornea. The actinic radiation sources 205-206 are used to fully cure the protective layer, solidifying the structure and preparing the prosthetic eye 230 for post-processing.

The printing system 221, in conjunction with the AI engine 222 and the cameras 225, creates a seamless and adaptive manufacturing environment for the prosthetic eye 230. The real-time monitoring and adjustments made by the AI engine 222 facilitate that each prosthetic eye produced is unique to the user's requirements, accurately replicating the appearance of their functional eye. This level of customization may particularly be beneficial for individuals requiring prosthetic eyes that match specific aesthetic or functional criteria.

In some embodiments, to create a natural look for the prosthetic eye 230, various techniques can be implemented in the additive manufacturing process 200A, particularly for features such as the vein patterns 211A (or pupil patterns) on the scleral portion of the prosthetic base 211. The vein patterns, which include fine, branching structures, can be produced by depositing a transparent polymerizable mixture around a colored polymerizable mixture designed to replicate the veins. This process begins with the precise deposition of a colored polymerizable mixture, such as red or blue pigments, using one or more print heads capable of emitting fine droplets of material. The colored polymerizable mixture forms the primary structure of the veins, accurately replicating their natural color and pattern.

The transparent polymerizable mixture is then deposited around and, in some cases, over the colored mixture to create a three-dimensional effect that enhances the realism of the veins. This transparent mixture may serve multiple purposes: it provides depth and texture to the vein patterns, creates a natural transition between the veins and the surrounding scleral region, and protects the pigmented layers from wear or damage. The deposition of the transparent mixture can occur immediately after the colored polymerizable mixture is deposited, or it can be applied as part of a subsequent layer, depending on the desired effect. The transparent mixture can also be formulated to have a slightly refractive index, creating a subtle light-scattering effect that mimics the way light interacts with natural blood vessels beneath the sclera.

Different types and colors of polymerizable mixtures can be emitted using the same or different print heads (201-202), depending on the complexity of the design and the capabilities of the printing system. In one embodiment, a single multi-material print head is used, equipped with separate nozzles or channels for each type of polymerizable mixture. For example, one nozzle may emit the red-colored polymerizable mixture for the veins, while another nozzle emits the transparent polymerizable mixture to encapsulate the veins. This configuration allows for seamless transitions between materials and facilitates that the mixtures are deposited in precise alignment with the prosthetic model 220.

In another embodiment, separate print heads may be used for each type of polymerizable mixture. For example, one print head may specialize in depositing pigmented materials for the veins, while a second print head deposits transparent or semi-transparent materials around the pigmented regions. The print heads operate in a coordinated manner, controlled by the AI engine 222, which dynamically adjusts their movement and emission rates based on real-time feedback from the cameras 225. This setup allows for greater flexibility in material selection and deposition strategies, enabling the creation of intricate and lifelike features.

For further customization, the deposition process may involve variable droplet sizes and flow rates to replicate the natural variability in vein thickness and branching. For example, larger droplets may be used to create the main branches of the vein patterns, while smaller droplets form finer capillary structures. The transparent polymerizable mixture can then be deposited with a slight overlap onto the pigmented regions to smooth transitions and add depth, creating the appearance of veins embedded beneath the scleral surface.

Unique effects can also be achieved by adjusting the curing process. For example, actinic radiation sources 205-206 may cure the pigmented and transparent layers at different rates, allowing the transparent material to settle slightly before curing fully. This technique can create a subtle convexity or texture around the veins, further enhancing their three-dimensional appearance. Additionally, varying the wavelength or intensity of the radiation may selectively cure certain layers, enabling fine-tuning of the optical and tactile properties of the finished prosthetic eye 230.

In another embodiment, the printing process may incorporate gradients within the transparent polymerizable mixture. By gradually transitioning from a clear mixture to a slightly tinted one, the veins can appear to fade naturally into the surrounding scleral region, mimicking the way real blood vessels transition beneath the surface of the eye. These gradients can be programmed into the digital value pattern of the prosthetic model 220, guiding the print heads to deposit the mixtures with precise spatial and material variations.

Furthermore, advanced patterns can be achieved by integrating micro-textures into the transparent polymerizable mixture. For example, the mixture may include nanoparticles or additives that scatter light, creating a slight iridescent effect around the veins. This feature not only enhances the visual realism of the prosthetic eye but also adds a dynamic quality as the appearance changes subtly with lighting conditions.

For applications requiring additional functionality, the transparent polymerizable mixture may also incorporate materials with specific optical or therapeutic properties. For example, UV-blocking agents can be added to protect the underlying pigmented layers from light-induced fading, or hydrophilic additives may be included to improve the moisture retention of the prosthetic eye. These enhancements can be tailored to the individual needs of the user, offering both aesthetic and functional benefits.

The described techniques for creating vein patterns and other natural features of the prosthetic eye 230 demonstrate the versatility and precision of the additive manufacturing process. By combining advanced material deposition strategies with real-time control and customization capabilities, the prosthetic eye can achieve a level of realism and functionality that closely replicates the appearance of a natural eye. These embodiments highlight the potential for innovation in ocular prosthetics, offering users highly personalized solutions that meet both aesthetic and practical requirements.

Referring now to FIG. 2B, another exemplary printing process 200B is illustrated for manufacturing an iris 212 and other detailed patterns on a pre-printed standard prosthetic base 211, according to some embodiments of the present invention. The standard prosthetic base 211 may be pre-fabricated using similar additive manufacturing techniques and stored in bulk, available in a variety of sizes and shapes to accommodate the diverse anatomical and aesthetic needs of different users. By utilizing standardized bases, the system allows for the rapid customization of prosthetic eyes, significantly reducing the time and material requirements for creating fully personalized ocular prostheses. The prosthetic base 211 may already include general features such as a smooth scleral region and predefined curvature, which serve as the foundation for further customization.

The process begins with the selection of an appropriate standard prosthetic base 211 based on the prosthetic model 220 generated for a specific user. The prosthetic model 220, which encapsulates the detailed anatomical and aesthetic data of the user's functional eye, is fed into the printing system 221. The printing system 221, which may include an AI engine 222, processes this data to guide the precise deposition of polymerizable mixtures 210 for constructing intricate features such as the iris 212, the pupil pattern 212A, and the vein pattern 211A on the selected prosthetic base 211. The integration of the AI engine 222 allows the system to dynamically adapt the printing parameters, such as the material flow rate, droplet size, and print head movement, to achieve a highly accurate replication of the prosthetic model 220.

The standard prosthetic base 211 may include features that enhance its compatibility with the customization process. For example, the base may have a pre-formed scleral region with slight grooves or textured areas to improve the adhesion of subsequent layers. The vein patterns 211A may be printed on the scleral region using fine droplets of pigmented polymerizable mixtures, such as red and blue ophthalmic-grade inks. These vein patterns are created to replicate the user's natural blood vessels, with variations in thickness and branching captured directly from the prosthetic model 220. A transparent polymerizable mixture may also be deposited around the vein patterns 211A to encapsulate and protect them, creating a seamless and lifelike transition between the veins and the surrounding scleral region.

Once the vein patterns 211A are completed, the printing system 221 transitions to constructing the iris 212, one of the most visually distinct components of the prosthetic eye 230. The iris 212 may be printed directly onto the standard prosthetic base 211 using a combination of colored and transparent polymerizable mixtures. The process involves the layer-by-layer droplet deposition of ophthalmic-grade CMYK pigments to recreate the intricate radial and concentric patterns of a natural iris. The pupil pattern 212A, which is positioned at the center of the iris 212, may be printed using an opaque black ink to replicate the appearance of a natural pupil. The AI engine 222 synchronizes the movement of the print heads 201 and 202 to precisely align the patterns with the underlying base, using real-time feedback from the cameras 225.

The cameras 225, which are strategically positioned around the printing enclosure 214, continuously capture high-resolution images of the printing process. These images are analyzed by the AI engine 222 to verify the alignment, accuracy, and consistency of the printed layers. If any discrepancies are detected, such as misalignment of the iris 212 with the prosthetic base 211, the AI engine 222 dynamically adjusts the deposition parameters to correct the issue. This real-time monitoring and adjustment capability allows for the creation of highly detailed and visually accurate prosthetic eyes.

In addition to the iris 212 and pupil pattern 212A, the printing process may include the application of a final protective layer over the customized components. This transparent layer not only protects the pigmented regions from environmental factors such as UV exposure and moisture but also provides a glossy finish that enhances the aesthetic appeal of the prosthetic eye 230. The protective layer may be printed using a transparent polymerizable mixture, which is cured using actinic radiation sources 205 and 206 to achieve a durable and stable finish.

The standard prosthetic base 211 also enables the integration of advanced functionalities into the prosthetic eye 230. For example, the base may include pre-designed cavities or grooves for embedding sensors, electronic components, or liquid reservoirs. During the customization process, the printing system 221 can deposit additional materials or devices into these cavities, guided by the prosthetic model 220. This approach allows for the creation of prosthetic eyes with features such as dynamic pupil movement, hydration mechanisms, or even sensor-based feedback systems.

In some embodiments, the printing process may utilize multiple print heads, each configured for a specific type of polymerizable mixture. For example, one print head may deposit pigmented inks for the iris 212, while another print head emits transparent materials for the protective layer. The use of specialized print heads allows for greater flexibility and precision in material deposition, enabling the system to achieve complex patterns and textures that closely mimic the natural eye.

The described process also supports the use of advanced techniques for enhancing the realism of the prosthetic eye 230. For example, gradient transitions between pigmented and transparent regions can be achieved by varying the material flow rates and deposition patterns. This technique may particularly be useful for creating subtle color transitions in the iris 212 or blending the vein patterns 211A into the scleral region. Additionally, the protective layer can be engineered to include micro-textures or optical coatings that replicate the light-reflective properties of a natural cornea.

The modular nature of the standard prosthetic base 211 simplifies the production and customization of prosthetic eyes, making it feasible to create high-quality prostheses at scale. By pre-fabricating bases in different sizes and shapes, the system reduces the time and material required for manufacturing while maintaining the ability to deliver fully personalized solutions. The combination of advanced additive manufacturing techniques, real-time AI-driven control, and user-specific customization facilitates that the final prosthetic eye 230 meets both aesthetic and functional requirements. This innovative approach represents a significant advancement in the field of ocular prosthetics, offering users highly realistic and adaptive solutions tailored to their individual needs.

Referring now to FIG. 2C, an exemplary printing process 200C is illustrated for manufacturing a prosthetic eye layer-by-layer droplet deposition using advanced additive manufacturing techniques, according to some embodiments of the present invention. The process begins with a prosthetic model 220, which is generated based on detailed scans of a user's functional eye or derived from pre-designed templates. The prosthetic model 220 encapsulates the anatomical and optical details required to replicate the appearance and structure of a natural eye. This digital model is fed into a printing system 221, which executes the layer-by-layer droplet deposition of one or more polymerizable mixtures 210 to form the prosthetic eye.

The printing system 221 may comprise a highly precise 3D printer equipped with a nozzle or print head capable of emitting polymerizable mixtures 210 in droplet form. These mixtures are deposited onto a receiving surface in a controlled manner, creating successive layers such as 210A and 210B. Each layer corresponds to a specific segment of the prosthetic eye's structure, including the scleral base, iris, pupil, and protective outer coatings. The printing system 221 operates under the guidance of the prosthetic model 220, so that each layer aligns with the intended design.

The polymerizable mixtures 210 used in the printing process are carefully formulated to achieve the desired mechanical, optical, and aesthetic properties of the prosthetic eye. These mixtures may include biocompatible monomers, cross-linking agents, and ophthalmic-grade pigments. For example, the layers forming the scleral portion may use an opaque white mixture, while the iris and pupil layers incorporate CMYK pigments to replicate the intricate color patterns of the user's functional eye. The polymerizable mixtures may also include additives to enhance their stability, viscosity, and curing properties.

After each layer is deposited, it is allowed to stabilize through controlled methods such as heat treatment, actinic radiation exposure, or UV curing. For example, the first layer 210A may undergo a brief exposure to UV light to initiate partial polymerization, creating a stable foundation for the deposition of the next layer 210B. This process, often referred to as pinning, prevents the material from spreading or deforming under the weight of subsequent layers. The actinic radiation sources integrated into the printing system 221 may include LEDs or laser emitters capable of emitting wavelengths tailored to the specific polymerizable mixtures being used.

The layer-by-layer droplet deposition approach depicted in FIG. 2C allows for a high degree of customization and precision in the manufacturing process. Each layer can be tailored to include specific details, such as vein patterns, iris textures, or surface contours. For example, the layer 210A may include grooves or recesses designed to accommodate vein patterns, which are subsequently filled with pigmented polymerizable mixtures in layer 210B. Similarly, the pupil may be constructed by depositing multiple concentric layers of opaque black material, achieving the desired depth and opacity.

The additive manufacturing process also enables the integration of functional enhancements into the prosthetic eye. For example, specific layers may include embedded sensors, micro-cameras, or dynamic pupil mechanisms. These components are added during the printing process, either by embedding them directly into the polymerizable mixture or by pausing the printing system 221 to place the components manually. The AI engine controlling the printing system 221 adjusts the deposition path and parameters to accommodate these additions, facilitating seamless integration with the surrounding material.

The precision and adaptability of the printing system 221 allow for the creation of complex geometric features, such as curved scleral regions or textured iris patterns. For example, the system can vary the droplet size and deposition rate to create fine details, such as radial lines in the iris or branching vein patterns in the sclera. The AI engine may use data from the prosthetic model 220 to optimize these parameters in real-time, so that the printed layers accurately replicate the user's functional eye.

Once the final layer of the prosthetic eye is printed, a comprehensive curing process is initiated to fully polymerize the material and stabilize the structure. This process may involve exposing the prosthetic eye to multiple wavelengths of actinic radiation, each targeting specific components of the polymerizable mixtures. For example, UV-A radiation may be used to cure transparent layers, while UV-B radiation targets pigmented regions to enhance color retention and durability. The curing process may also include heat treatment to improve the mechanical properties of the prosthetic eye, such as its flexibility and resistance to wear.

In some embodiments, the printing system 221 incorporates feedback mechanisms to monitor the quality of the printing process. Sensors and cameras integrated into the system capture real-time data on droplet placement, layer alignment, and material flow. This data is analyzed by the AI engine, which adjusts the printing parameters as needed to correct any deviations from the prosthetic model 220. For example, if the system detects that a droplet of polymerizable mixture 210 has been misplaced, it can modify the trajectory of subsequent droplets to compensate for the error.

The described process allows for the creation of prosthetic eyes with unparalleled realism and functionality. By combining advanced materials, precise deposition techniques, and real-time monitoring, the system achieves a level of customization that meets the unique needs of each user. The layer-by-layer droplet deposition approach not only replicates the appearance of a natural eye but also provides a robust foundation for incorporating advanced features, such as hydration mechanisms or dynamic pupil movement.

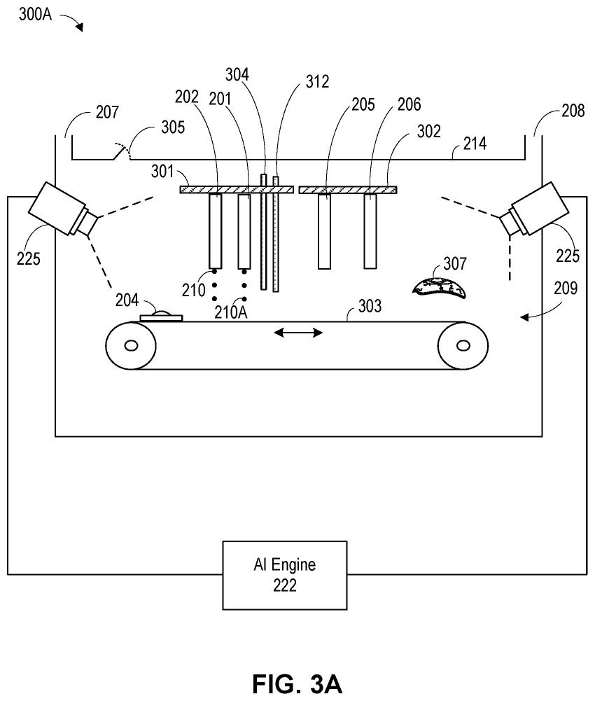

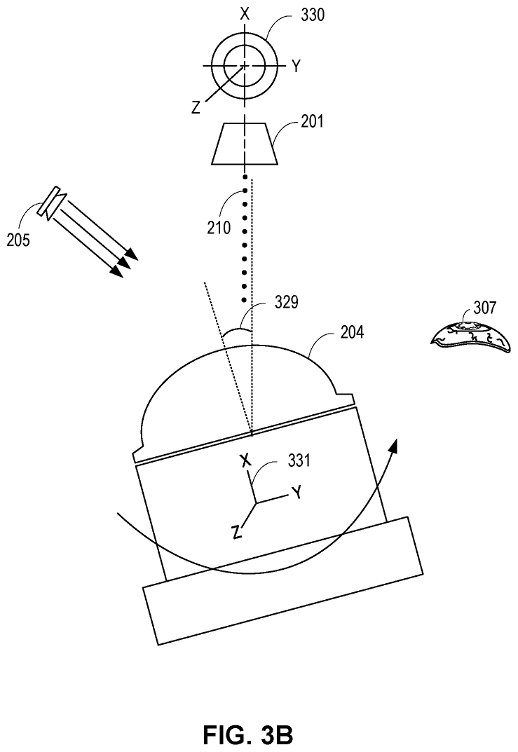

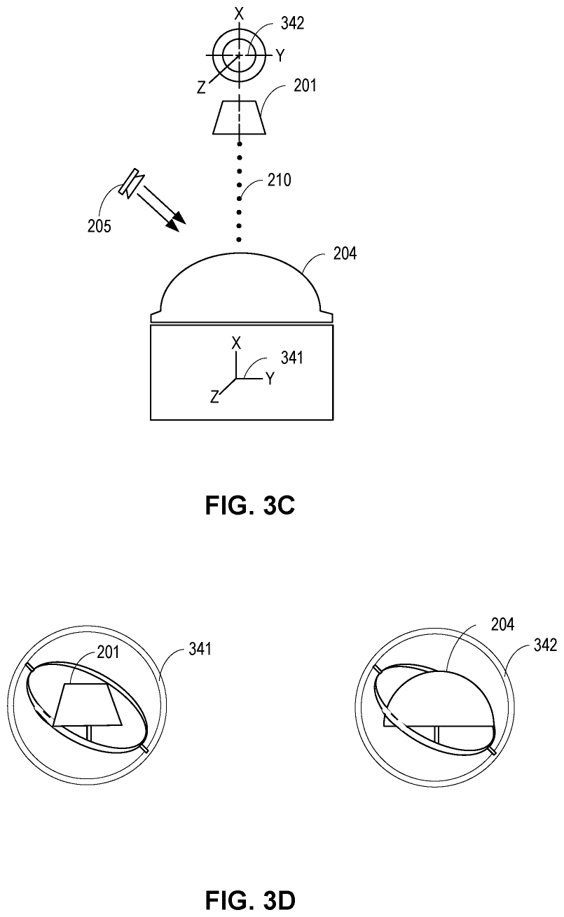

Referring now to FIG. 3A, a schematic illustration of an alternative 3D printing system 300A is illustrated, showcasing various components and processes used to manufacture a prosthetic eye 307 through additive manufacturing, according to some embodiments of the present invention. The system 300A includes key components such as printheads 201 and 202, actinic radiation sources 205 and 206, a substrate 204, and an enclosure 214, all integrated into a controlled environment facilitated by ports 207 and 208. The system may be designed to deposit and cure polymerizable mixtures 210 layer-by-layer droplet deposition, forming the prosthetic eye 307 with exceptional precision and customization.

The substrate 204 serves as the foundational support for the printing process and features a smooth receiving surface that can accommodate the deposition of the polymerizable mixture 210. During the process, the substrate 204 may be moved relative to the printheads 201 and 202 using an actuation structure 303, such as a belt drive or linear stepper motor system. This movement is synchronized with the deposition of material to enable precise control of the additive manufacturing process. The receiving surface of the substrate 204 may be pre-treated with release agents or textured to facilitate the eventual removal of the prosthetic eye 307 without damage.

The printheads 201 and 202 are capable of emitting droplets 210 of polymerizable mixtures in highly controlled patterns. These mixtures may vary in composition to produce different layers of the prosthetic eye 307, including structural layers, aesthetic features such as iris and vein patterns, and functional coatings. For example, the printhead 201 may emit an opaque white mixture to form the scleral base, while the printhead 202 deposits pigmented mixtures to create the intricate radial and concentric patterns of the iris. Additionally, transparent polymerizable mixtures may be deposited over the pigmented layers to enhance depth and provide a natural glossy appearance. The printheads 201 and 202 may also be moved using the respective actuator 301.

The actinic radiation sources 205 and 206 are strategically positioned to cure the deposited polymerizable mixture 210 in real-time. These sources may emit radiation at specific wavelengths, such as UV-A or UV-B, depending on the curing requirements of the material being deposited. The radiation sources are mounted on actuators 302, enabling precise movement and alignment with the substrate 204 to selectively cure specific regions of the prosthetic eye 307. This selective curing capability allows for the incorporation of complex features, such as textured surfaces or embedded components, without affecting adjacent areas.

The enclosure 214 creates a controlled atmosphere 209, which is important for maintaining the quality and consistency of the printed prosthetic eye 307. The atmosphere within the enclosure 214 can be regulated through ports 207 and 208, which allow the introduction of gases such as nitrogen to control oxygen levels. This regulation minimizes the impact of oxygen on the polymerization process, so that the material cures uniformly and retains its intended properties. Oxygen sensors 304 integrated into the system provide real-time feedback to the AI engine 222, which adjusts the atmosphere 209 as needed. Oxygen inlet 305 may be opened or closed based on the oxygen sensors 304 to adjust the atmosphere 209.

The system 300A also includes a UV-blocking screen 312, which can be deployed to shield specific areas of the prosthetic eye 307 from unintended exposure to actinic radiation. This screen may particularly be useful when curing selective layers or regions, allowing for greater control over the polymerization process. For example, the screen 312 can block UV light from reaching the scleral base while curing the iris layers, preserving the distinct optical properties of each region.

An AI engine 222 manages the overall operation of the system 300A, analyzing data from cameras 225 and other sensors to guide the printing and curing processes. The cameras 225 capture high-resolution images and videos of the manufacturing process, providing real-time feedback on droplet placement, layer alignment, and material distribution. If any discrepancies are detected, such as misalignment of layers or inconsistent curing, the AI engine 222 adjusts the movement of the printheads 201 and 202, the substrate 204, or the actinic radiation sources 205 and 206 to correct the issue.

The actuation structure 303 facilitates the movement of components relative to each other, enabling the system to print complex geometries and intricate details. For example, the substrate 204 can be moved in precise increments to allow the deposition of micro-patterns, such as vein structures or textured scleral regions. Similarly, the printheads 201 and 202 can move independently to deposit multiple materials in a single pass, streamlining the manufacturing process.

The curing process is an important step in the formation of the prosthetic eye 307. After sufficient layers of polymerizable mixture 210 have been deposited, the actinic radiation sources 205 and 206 expose the material to light, initiating the polymerization reaction. The exposure parameters, such as wavelength, intensity, and duration, are optimized for each layer to achieve the desired mechanical and optical properties. For example, the scleral base may require longer curing times to enhance its durability, while the iris layers may be cured at lower intensities to preserve their color vibrancy.

Once the prosthetic eye 307 is fully formed, it is carefully released from the substrate 204. This release process may involve soaking the substrate and prosthetic eye in a solution, such as buffered saline, to weaken the adhesion between the layers and the substrate. Alternatively, mechanical methods such as gentle peeling or lifting may be used. The released prosthetic eye 307 undergoes additional post-processing steps, including cleaning, sterilization, and hydration, to prepare it for use.

Referring now to FIG. 3B, a schematic diagram illustrates mechanisms for introducing tilt into an additive manufacturing system, enabling precise control over the deposition and alignment of polymerizable mixture droplets 210 to form a prosthetic eye 307, according to some embodiments of the present invention. The system includes a printhead 201 positioned above a receiving substrate 204, with both components capable of dynamic adjustments to achieve specific topographical and structural requirements during the additive manufacturing process.

In the depicted arrangement, the printhead 201 emits droplets 210 of polymerizable mixture in a controlled manner to form successive layers on the receiving substrate 204. The receiving substrate 204 may be mounted on a tiltable platform controlled by a tilt mechanism 330-331, which allows precise adjustments along the X, Y, and Z axes. The tilt 329 introduced by the mechanism facilitates accurate alignment of the droplets 210 with the predefined design parameters for the prosthetic eye 307. This adjustment may particularly be beneficial when creating complex features such as curved scleral regions or intricate vein patterns on the scleral portion.

The tilt mechanism 330-331 may include electro-mechanical devices such as servos, gimbals, or stepper motors, capable of micrometer-level adjustments. These mechanisms are synchronized with the deposition process to introduce the desired tilt 329 after each pass of the printhead 201 relative to the substrate 204. For example, after depositing a layer of material corresponding to the scleral base, the tilt mechanism 330-331 can adjust the substrate 204's position to enable the accurate deposition of subsequent layers for the iris or vein patterns.

The actinic radiation source 205 may be strategically positioned to cure the polymerizable mixture droplets 210 as they are deposited on the substrate 204. This source emits radiation at specific wavelengths tailored to the curing requirements of the material, such as UV-A or UV-B light. The precise placement of the radiation source 205 relative to the substrate 204 and the printhead 201 allows for selective curing of the deposited material, so that each layer retains its shape and integrity while bonding seamlessly with the next.

The additive manufacturing process illustrated in FIG. 3B allows for the creation of prosthetic eyes 307 with highly customized features. For example, the system can replicate the natural curvature and texture of a user's eye by depositing successive layers of polymerizable mixture in a pattern guided by the prosthetic model. The tilt 329 introduced by the mechanism 330-331 enables the creation of non-planar geometries, such as the curved scleral region and the domed iris area, which are important for achieving a realistic appearance.

In some embodiments, the tilt adjustment may be achieved through software modifications to the deposition pattern rather than physical movement of the substrate 204. The software, controlled by an AI engine, analyzes the prosthetic model and adjusts the droplet trajectory and deposition parameters to compensate for the desired tilt. This approach may particularly be useful when manufacturing prosthetic eyes with highly intricate patterns, as it allows for precise control without requiring complex mechanical adjustments.

The system may also include cameras and sensors for real-time monitoring and feedback during the manufacturing process. High-resolution cameras capture images of the deposited material, which are analyzed by the AI engine to verify alignment and layer consistency. If discrepancies are detected, the AI engine can adjust the tilt 329, printhead 201 position, or deposition parameters to correct the issue. This continuous feedback loop ensures the accuracy and quality of the prosthetic eye 307.

The described process enables the integration of advanced features into the prosthetic eye, such as dynamic pupil mechanisms or hydration systems. For example, specific layers may include embedded channels or cavities designed to hold liquid reservoirs or micro-actuators. The tilt mechanism 330-331 facilitates the precise deposition of these functional components, facilitating that they are correctly aligned with the surrounding structure.

Referring now to FIG. 3C, a schematic diagram illustrates a printhead 201 positioned relative to a receiving substrate 204, with droplets 210 of polymerizable mixture being dispensed in the presence of an actinic radiation source 205. The arrangement incorporates mechanisms for introducing controlled movements or inclinations to either the printhead 201 or the substrate 204 through actuators 341 and 342, respectively. This configuration provides precise control over the deposition process, enabling the creation of a prosthetic eye with highly detailed and accurate features.

The printhead 201, guided by a printhead camber actuator 342, can move along or tilt relative to the X, Y, and Z axes, as shown in the diagram. This movement allows the printhead 201 to adjust the trajectory of the droplets 210 being emitted, aligning them with the intended deposition pattern. Such adjustments are particularly beneficial when printing curved or contoured surfaces, such as the scleral base or the iris dome of a prosthetic eye. The actuator 342 may comprise a servo motor, stepper motor, or piezoelectric device capable of sub-micron-level precision, facilitating that the droplets 210 are deposited exactly as dictated by the prosthetic model.

The substrate 204, on which the droplets 210 are received, may be mounted on a substrate camber actuator 341. This actuator allows the substrate 204 to tilt or move relative to the printhead 201, providing additional flexibility in aligning the deposited material with the desired topography of the prosthetic eye. For example, the substrate 204 can be inclined to match the curvature of the scleral region, allowing for a uniform deposition of material without the need for complex printhead adjustments. The combination of printhead and substrate movements, controlled by actuators 342 and 341, respectively, enables the system to achieve a high degree of precision in layer alignment and material distribution.

The droplets 210 of polymerizable mixture are dispensed in a controlled stream from the printhead 201. These droplets are deposited onto the substrate 204 to form successive layers, each contributing to the final structure of the prosthetic eye. The material used for the droplets 210 may vary depending on the specific layer being formed. For example, opaque white polymerizable mixtures may be used for the scleral base, while colored mixtures are employed for the iris and vein patterns. The consistency and flow rate of the droplets 210 are dynamically adjusted by the printing system to maintain alignment and achieve the desired thickness and texture of each layer.

The actinic radiation source 205 may be positioned to expose the droplets 210 to light as they are deposited, initiating a polymerization reaction that stabilizes the material. This process, known as pinning, partially cures the material to prevent it from spreading or deforming, creating a stable foundation for subsequent layers. The wavelength and intensity of the radiation emitted by the source 205 can be adjusted based on the curing requirements of the polymerizable mixture. For example, UV-A light may be used for transparent layers, while UV-B light is suitable for pigmented regions. The radiation source 205 may also move in synchronization with the printhead 201 to maintain consistent exposure across the substrate 204.

The system's ability to tilt the printhead 201 and the substrate 204 independently allows for the creation of complex geometries and detailed features. For example, the iris of the prosthetic eye, which may include intricate radial and concentric patterns, can be printed with exceptional accuracy by adjusting the inclination of the printhead 201 to match the curvature of the iris dome. Similarly, vein patterns on the scleral region can be deposited with varying thicknesses and branching angles by tilting the substrate 204 to optimize the droplet placement.

The integration of actuators 341 and 342 into the additive manufacturing system also facilitates the incorporation of advanced functionalities into the prosthetic eye. For example, cavities or channels designed to hold liquid reservoirs or electronic components can be precisely positioned by tilting the substrate 204 to expose the target area to the printhead 201. This capability enhances the customization and functionality of the prosthetic eye, making it suitable for a wide range of user needs.

The described system can also adapt to variations in material properties or environmental conditions. For example, the AI engine controlling the manufacturing process can analyze real-time data from sensors and cameras to detect deviations in material flow or curing consistency. If an issue is identified, the AI engine can adjust the movements of the printhead 201 or the substrate 204 to compensate, facilitating that the final prosthetic eye meets the specified quality standards.

In addition to its application in printing prosthetic eyes, the system depicted in FIG. 3C may be used for other medical or optical devices requiring high precision and customization. The ability to control the tilt and alignment of the printhead and substrate makes it suitable for producing contact lenses, camera lenses, or medical imaging devices with complex geometries and optical properties.

Referring now to FIG. 3D, a schematic illustration is provided for electro-active devices configured to impart camber movement or tilt to one or both of a printhead 201 and a receiving substrate 204, according to some embodiments of the present invention. The depicted camber actuators 341 and 342 are representative of electro-mechanical systems, such as servos or gimbals, which allow for multi-directional adjustments along various axes to facilitate precise alignment and deposition of material in the additive manufacturing process.

The substrate camber actuator 341 is shown as a mechanism capable of tilting the receiving substrate 204 in multiple directions, enabling the substrate 204 to adjust its orientation relative to the printhead 201. Such movement may particularly be advantageous when printing prosthetic eyes, as the spherical geometry and varying contours of the scleral base and iris require precise alignment for accurate material deposition. For example, the camber actuator 341 may tilt the substrate 204 along the X and Y axes while maintaining a stable Z position to achieve optimal curvature during the printing of the scleral region or vein patterns.

Similarly, the printhead camber actuator 342 is depicted as a system capable of tilting the printhead 201 relative to the substrate 204. This functionality enables the printhead 201 to adjust its orientation dynamically, facilitating that the emitted droplets of polymerizable mixture reach their intended target on the substrate 204. The printhead camber actuator 342 may also facilitate angular adjustments to compensate for any deviations in the substrate's position, maintaining the integrity of the printed patterns and layers.

In some embodiments, the camber actuators 341 and 342 may be implemented as gimbal systems, providing full rotational freedom along all three axes. Such systems allow for precise and independent adjustments to both the printhead 201 and the substrate 204, enabling the additive manufacturing process to accommodate complex geometries and intricate designs. For example, when printing the iris of a prosthetic eye, the gimbal mechanism may rotate the printhead 201 to deposit concentric rings of colored material while tilting the substrate 204 to match the curvature of the iris dome.

Alternatively, the camber actuators 341 and 342 may utilize advanced electro-active materials, such as shape memory alloys or piezoelectric actuators, to achieve the desired movement. These materials offer the advantage of compactness and high precision, making them suitable for applications where space constraints or fine adjustments are required. For example, a shape memory alloy-based actuator may provide minute tilting movements to the substrate 204 during the deposition of vein patterns, allowing for seamless integration of these features into the prosthetic eye.

The electro-active devices depicted in FIG. 3D are also compatible with real-time monitoring and feedback systems. Cameras and sensors integrated into the manufacturing setup can capture high-resolution data on the alignment and movement of the printhead 201 and the substrate 204. This data is processed by an AI engine, which analyzes the positional accuracy and makes adjustments to the camber actuators 341 and 342 as needed. For example, if the AI detects a misalignment between the printhead 201 and the substrate 204, it can command the camber actuators to correct the orientation, providing precise material deposition.

The incorporation of camber movement mechanisms into the additive manufacturing system enables the creation of prosthetic eyes with unparalleled realism and functionality. By allowing for dynamic adjustments during the printing process, these mechanisms facilitate that each layer of the prosthetic eye is accurately aligned with the intended design. For example, when printing the scleral base, the substrate camber actuator 341 can tilt the receiving surface to match the natural curvature of the sclera, resulting in a more anatomically accurate prosthetic eye. Similarly, the printhead camber actuator 342 can adjust the angle of material deposition to create intricate features, such as vein patterns or textured surfaces, without compromising the overall design.

In some embodiments, the electro-active devices may also be used to incorporate advanced features into the prosthetic eye. For example, the substrate camber actuator 341 may tilt the substrate 204 to create cavities or channels for embedding electronic components, such as sensors or micro-cameras. The printhead camber actuator 342, on the other hand, may adjust the orientation of the printhead 201 to deposit materials with different viscosities or optical properties, enabling the integration of dynamic pupil mechanisms or hydration systems into the prosthetic eye.

The described system also offers scalability and versatility, making it suitable for various applications beyond prosthetic eyes. For example, the same principles may be applied to the production of contact lenses, camera lenses, or medical imaging devices, where precise control over geometry and material properties is important. By leveraging the capabilities of the camber actuators 341 and 342, the additive manufacturing system can produce optical devices with complex designs and high precision.

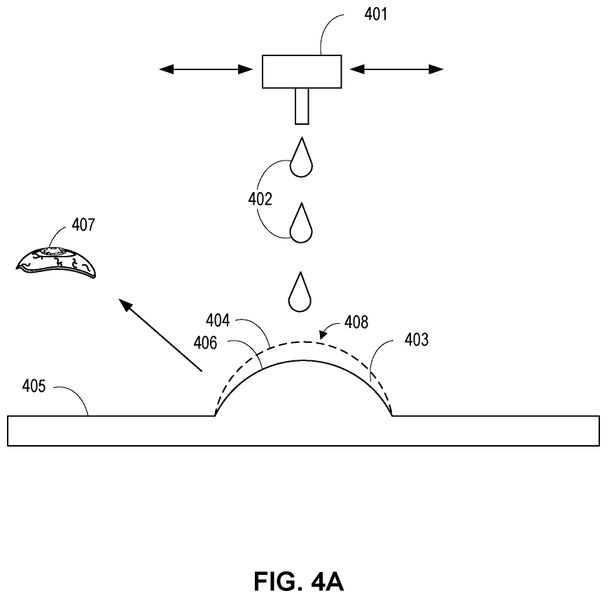

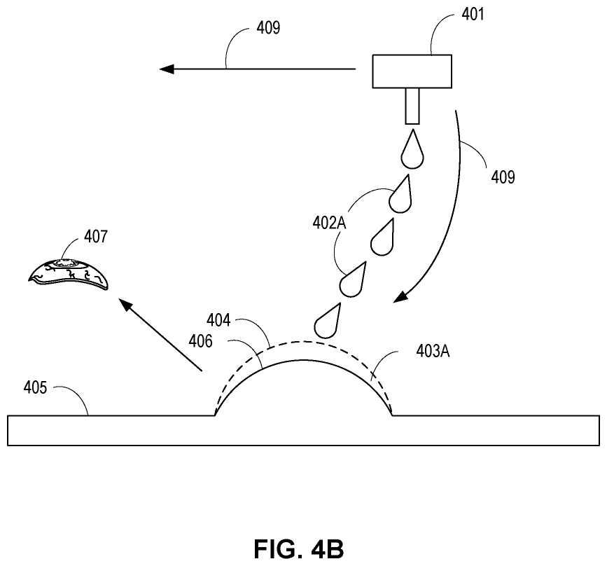

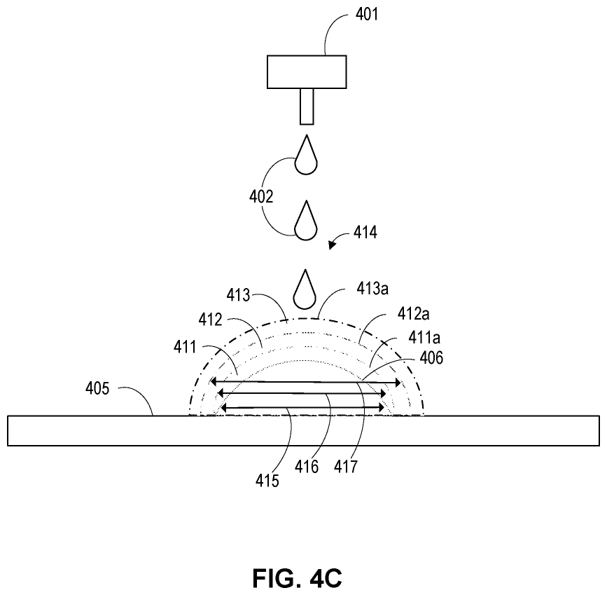

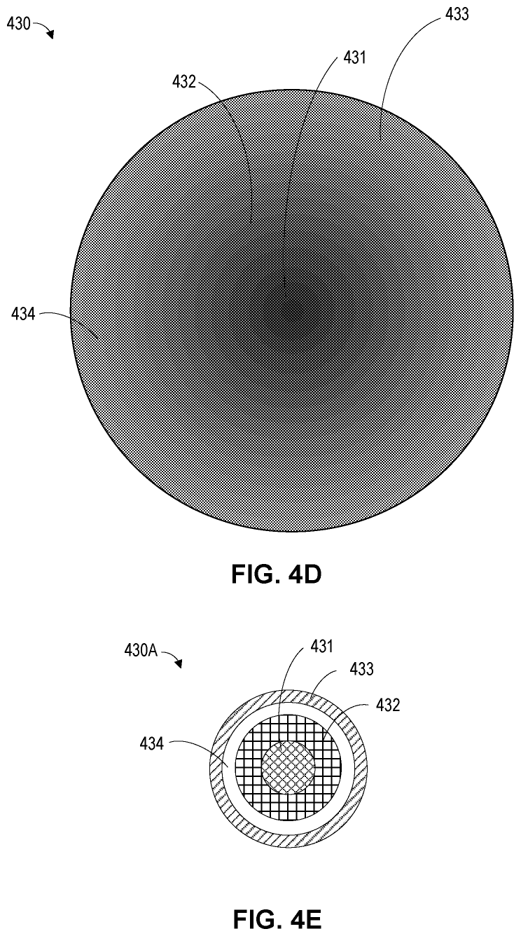

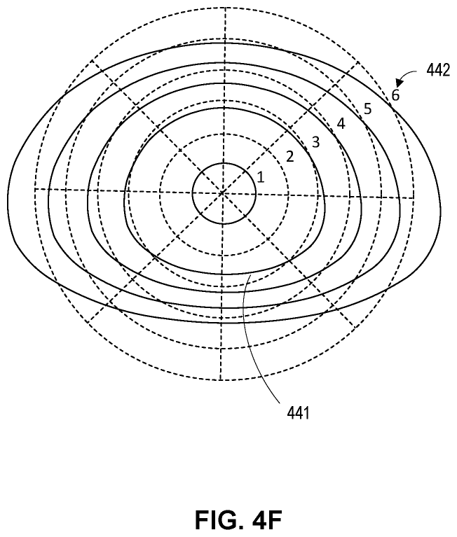

Referring now to FIG. 4A, a schematic diagram illustrates an additive manufacturing process in which a printhead 401 dispenses droplets 402 of polymerizable mixture onto a receiving surface 404 of a substrate 405 to form a prosthetic eye 407. The process demonstrates a precise deposition of the polymerizable mixture 402, controlled by a mathematical model that guides the printhead 401 in relation to the substrate 405.

The printhead 401 moves along an X-Y axis plane above the substrate 405, dispensing droplets 402 of polymerizable mixture in a controlled trajectory. Each droplet 402 is directed toward specific positions on the receiving surface 404 of the substrate 405 based on a pre-defined two-dimensional print pattern. The print pattern, derived from an energy intensity pattern or map, may dictate the volume and type of polymerizable mixture 402 to be deposited at each X-Y position, allowing the printed structure to achieve the desired shape and optical properties. For example, the numerical values in the print pattern may correspond to areas requiring more or less light to pass through the prosthetic eye 407, determining the density and color intensity of the printed material.

As droplets 402 accumulate, a three-dimensional volume of polymerizable mixture 403 begins to form on the receiving surface 404. The volume 403 gradually conforms to the intended geometry of the prosthetic eye 407, including the scleral base, the curvature of the iris, and any embedded vein patterns. The apex 408 of the deposited volume 403, representing the highest point of the structure, serves as a reference for perpendicular alignment with the printhead 401. This perpendicular trajectory facilitates precise droplet placement, reducing the likelihood of aberrations or uneven layering.

The receiving area 406 of the substrate 405 provides an initial foundation for the deposition process. This area is pre-treated to optimize adhesion and smoothness, facilitating that the polymerizable mixture 402 forms a consistent base layer. Over successive passes of the printhead 401, the receiving surface 404 transitions from the substrate 405 to the previously deposited layers of polymerizable mixture 403. This progression allows for the seamless integration of new material with existing layers, creating a unified structure without visible striations or interlayer artifacts.

To enhance the optical qualities of the prosthetic eye 407, the surface of the polymerizable mixture 403 is subject to natural smoothing forces before curing. These forces, including Van der Waals interactions, ion-dipole bonding, and hydrogen bonding, act on the surface 404 to reduce interstitial irregularities and create a polished appearance. For example, the apex 408 of the volume 403 benefits from these forces, achieving a smooth curvature that closely mimics the natural anatomy of a human eye.

Pinning or partial curing of the polymerizable mixture 403 occurs at intervals during the deposition process. An actinic radiation source (not shown) may emit UV or visible light to stabilize the material, preventing unwanted spreading or deformation while maintaining its capacity to bond with subsequent layers. This intermediate stabilization process may particularly be useful for creating detailed features, such as the fine veins embedded within the scleral base or the intricate radial patterns of the iris.

The final curing step involves exposing the fully formed volume of polymerizable mixture 403 to actinic radiation or heat. This process induces a complete polymerization reaction, transforming the liquid material into a solid, biocompatible structure. The cured prosthetic eye 407 exhibits the desired mechanical properties, including flexibility, durability, and optical clarity, making it suitable for long-term wear.

In some embodiments, the printing process incorporates advanced customization features. For example, the printhead 401 may dispense multiple types of polymerizable mixtures 402 simultaneously, each tailored to a specific function or aesthetic requirement. A translucent mixture may be used for the scleral base, while pigmented mixtures create the colored patterns of the iris. Additionally, specialized mixtures containing functional additives, such as sensors or hydration channels, may be integrated into the design.

The prosthetic eye 407, once printed, undergoes further post-processing to enhance its usability and comfort. For example, the eye may be immersed in a hydration solution to achieve its final dimensions and flexibility. Optional sterilization and packaging steps prepare the prosthetic eye for distribution and use.

Referring now to FIG. 4B, the diagram illustrates a dynamic printing process where a printhead 401 dispenses droplets of polymerizable mixture 402A along a droplet trajectory 409, relative to a receiving surface 404 of a substrate 405. The printing path of the printhead 401 is represented by bidirectional arrows 409, indicating movement across the substrate 405. The droplet trajectory 409 is determined by the velocity, direction, and angular orientation of the printhead 401 during the deposition of polymerizable mixture 402A. This process provides a continuous and controlled integration of the deposited material with previously formed layers of polymerizable mixture 403A.

The deposition process begins with the printhead 401 releasing droplets of polymerizable mixture 402A at specific intervals and angles, informed by a pre-defined energy intensity pattern. The trajectory 409 of the droplets may be calculated to align with the curvature of the receiving surface 404, including the apex of the formed volume of polymerizable mixture 403A. The trajectory 409 may particularly be advantageous for forming complex structures, such as the curved scleral base or domed iris of the prosthetic eye 407. The angular alignment of the droplets relative to the receiving surface 404 minimizes the risk of misalignment, thereby achieving a seamless integration of the newly deposited material.

As the droplets 402A are deposited, they merge and meld into the previously deposited polymerizable mixture 403A. This melding process occurs due to the compatibility of the polymerizable material and the precision of the droplet trajectory 409. The integration is enhanced by surface tension forces and the material's rheological properties, which promote uniform spreading and bonding at the molecular level. This process differs from conventional additive manufacturing techniques, as the droplets do not form discrete, isolated layers but instead contribute to a unified structure with no discernible striations.

To stabilize the deposited material, actinic radiation sources (not shown) may expose the polymerizable mixture 402A to ultraviolet (UV) or visible light, initiating a gelation or pinning process. The pinned material retains its shape and positional accuracy while remaining receptive to subsequent layers of droplets. This iterative process of deposition, melding, and pinning may be repeated until the desired geometry of the prosthetic eye 407 is achieved. For example, the process may be used to create intricate vein patterns within the scleral base, facilitating that these patterns are seamlessly integrated with the surrounding material.

The dynamic movement of the printhead 401 along the path 409 offers flexibility in adapting the printing process to various design requirements. For example, the printhead 401 may accelerate or decelerate along the path 409 to adjust the droplet deposition rate, creating regions with varying material densities or optical properties. This capability may particularly be useful for printing the iris and pupil of the prosthetic eye 407, where precise color gradations and transparency levels are required to replicate a natural appearance.

In some embodiments, the printhead 401 may operate at an angle other than a right angle to the receiving surface 404, as shown in FIG. 4B. This angular deposition technique allows the droplets 402A to follow a curved trajectory 409, enabling the creation of complex geometries and features. For example, the printhead 401 may be tilted to deposit material along the contours of the scleral base, facilitating that the curvature matches the anatomical structure of the eye socket.

The receiving surface 404, initially provided by the substrate 405, transitions to the previously deposited volume of polymerizable mixture 403A as the printing process progresses. This transition is managed seamlessly, with the droplets 402A adhering to both the substrate 405 and the gelled material of the prior layers. This capability allows for the gradual construction of the prosthetic eye 407, layer by layer with deposited patterns of droplets, with each layer contributing to the overall structural integrity and aesthetic quality of the final product.

The described process may be further enhanced by incorporating multiple printheads 401, each dispensing a different type or color of polymerizable mixture 402A. For example, one printhead may deposit a translucent mixture for the scleral base, while another deposits pigmented material for the iris or vein patterns. The coordination of these printheads may be managed by an AI-controlled system, which synchronizes the deposition rates, angles, and trajectories to achieve a cohesive and realistic prosthetic eye.

The prosthetic eye 407, once fully formed, undergoes a curing process to solidify the polymerizable mixture 403A. This step involves exposure to controlled actinic radiation or heat, which completes the polymerization reaction and transforms the material into a durable, biocompatible structure. The final prosthetic eye 407 exhibits the desired mechanical and optical properties, making it suitable for extended wear and aesthetic purposes.

Referring now to FIG. 4C, the figure illustrates a detailed additive manufacturing process for creating a prosthetic eye through successive deposition events using a printhead 401 and a substrate 405 with a receiving area 406. The deposition process comprises multiple sequential events where polymerizable mixture 402 may be deposited to form overlapping and melded layers 411, 412, and 413. Each deposition event creates a corresponding layer, with distinct receiving surfaces 411a, 412a, and 413a, which serve as a base for the next layer.

The printhead 401 dispenses polymerizable mixture 402 in droplet form during each deposition event, guided by a mathematical model of the prosthetic eye design. The trajectory 414 of the droplets may be precisely calculated to align with the contours of the receiving area 406 or the previously deposited surface (e.g., 411a, 412a, or 413a). This alignment is important for achieving a smooth and uniform structure. The printhead 401 may be controlled to move laterally or rotationally relative to the substrate 405 to cover the entire intended footprint of the prosthetic eye. The droplets merge seamlessly with the previously deposited material, forming a unified volume without discernible striations or artifacts.

During the first deposition event, polymerizable mixture 411 may be emitted from the printhead 401 to form the foundational layer. The receiving area 406 of the substrate 405 acts as the base, providing an initial surface for the polymerizable mixture 411 to adhere to. The receiving surface 411a of this layer becomes the base for the second deposition event, during which polymerizable mixture 412 is deposited. The second deposition event further refines the structure by adding to the back curve diameter 415 and enhancing the prosthetic eye's contours. Subsequent deposition events, such as the third event forming polymerizable mixture 413, continue to build the structure layer by layer, with the corresponding receiving surfaces (412a, 413a, etc.) serving dual roles as both the back curve and the foundation for subsequent layers.

Each layer's deposition may be followed by a gelation or pinning process, achieved by exposing the deposited polymerizable mixture 411, 412, or 413 to actinic conditions, such as UV light or heat. This process stabilizes the material, preventing undesired spreading or deformation while maintaining its compatibility with additional layers. For example, actinic radiation may be applied after the first deposition event to partially polymerize mixture 411, creating a stable yet receptive surface 411a. The same process may be repeated after each subsequent deposition event to maintain structural integrity.

The cumulative result of these successive deposition events is the formation of a prosthetic eye 414 with precise geometric and optical properties. The back curve diameters 415, 416, and 417 increase incrementally with each layer, allowing for the creation of a natural curvature that mimics the anatomical shape of a human eye. The ability to control the dimensions and alignment of each layer provides exceptional accuracy in replicating the user's functional eye or achieving specific aesthetic and functional requirements.

In some embodiments, the deposition process involves multiple types of polymerizable mixtures dispensed from the printhead 401. For example, a translucent mixture may be used for the foundational layers to form the scleral base, while pigmented mixtures are applied in later layers to create the iris and vein patterns. These mixtures may be dispensed from a single printhead 401 or multiple printheads operating in unison. The incorporation of pigmented mixtures into specific layers, such as 412 or 413, allows for the creation of detailed and realistic patterns that enhance the prosthetic eye's visual appeal.

The final curing step follows the completion of all deposition events. The prosthetic eye 414 may be exposed to actinic radiation or heat to complete the polymerization process, transforming the deposited polymerizable mixture into a solid, durable, and biocompatible structure. The curing process also eliminates any residual stresses within the material, facilitating that the final prosthetic eye is free of distortions and artifacts.

In some embodiments, additional deposition events may be performed to apply a transparent protective layer over the prosthetic eye's surface. This layer, deposited as polymerizable mixture 416 or 417, provides a smooth finish and enhances the optical clarity of the iris and pupil patterns. The protective layer also contributes to the prosthetic eye's durability, making it resistant to wear and tear during prolonged use.

Referring now to FIG. 4D, the figure illustrates an exemplary energy intensity pattern represented as a grayscale image 430, which may be utilized to control the deposition of polymerizable mixture during the additive manufacturing process to print the iris portion of a prosthetic eye. The grayscale image 430 comprises varying shades, ranging from lighter areas 434 to darker areas 431, with gradients represented as mid-tone regions 432 and 433. Each shade corresponds to specific deposition instructions that regulate the amount and type of polymerizable mixture deposited at a particular location on a receiving surface.

The grayscale image 430 serves as a visual and computational map that translates pixel values into additive manufacturing control commands. These pixel values may either directly represent or be converted into numerical data, such as integers or floating-point values, associated with the thickness and intensity of the deposit at each location. For example, darker areas 431 in the grayscale image 430 represent regions requiring thicker deposits of polymerizable mixture, while lighter areas 434 correspond to thinner deposits. Mid-tone regions 432 and 433 indicate intermediate thicknesses that seamlessly transition between the darker and lighter areas.

The printing system interprets the pixel data of the grayscale image 430 to generate control commands for the printhead. These commands direct the printhead to deposit polymerizable mixture in precise amounts and patterns that replicate the grayscale image 430. For example, a pixel value associated with a darker area 431 may command the printhead to emit a greater volume of polymerizable mixture to form a thicker deposit. Conversely, a pixel value for a lighter area 434 may instruct the printhead to release a smaller volume of polymerizable mixture, resulting in a thinner deposit.

In one embodiment, the printhead executes these commands during successive passes over the receiving surface, building up layers of polymerizable mixture to form the iris's detailed structure. For example, the printhead may deposit a relatively larger amount of material for the thicker deposit 431 during one pass, followed by depositing a relatively smaller amount for the thinner deposit 434 in adjacent areas. The seamless melding of these deposits creates a natural gradient that replicates the intricate patterns and shading of a human iris.

The energy intensity pattern represented by the grayscale image 430 enables the precise recreation of optical properties, such as light absorption and reflection, within the prosthetic eye. For example, the thicker deposit 431 may correspond to regions of the iris designed to reduce light transmission, mimicking the darker pigmentation of a natural eye. Similarly, the thinner deposit 434 may correspond to lighter, more translucent areas of the iris. This level of precision allows for the creation of prosthetic eyes with highly realistic aesthetics and functional light transmissibility or reflectivity.

In another embodiment, the grayscale image 430 may be modified dynamically during the printing process based on real-time feedback from sensors or cameras monitoring the deposition. For example, if the system detects deviations in the thickness of the deposited material, the grayscale values may be adjusted, and new control commands may be generated to correct the discrepancies. This feedback loop, managed by an AI engine integrated into the printing system, provides accurate replication of the desired iris pattern.

The grayscale image 430 can also be customized for each user, allowing for the creation of personalized prosthetic eyes. For example, a scanned image of the user's functional eye may be converted into a grayscale energy intensity pattern, with pixel values corresponding to the unique pigmentation and patterns of the iris. This customized grayscale image 430 is then used to generate control commands for printing a prosthetic eye that closely resembles the user's natural eye.

In some embodiments, the grayscale image 430 may incorporate additional layers of information, such as vein patterns or scleral features, enabling the printing system to create a complete prosthetic eye with integrated details. For example, separate grayscale maps may be used for the iris, pupil, and sclera, each guiding the deposition of polymerizable mixtures specific to those regions. This multi-map approach allows for the simultaneous printing of complex structures with varying material properties and thicknesses.

Referring now to FIG. 4E, the figure illustrates an enhanced energy intensity pattern 430A represented as a grayscale image that may guide additive manufacturing processes for creating a prosthetic eye, an iris structure of a prosthetic eye, or other related components. The grayscale image 430A incorporates various zones, including a thicker deposit region 431, an upper surface gradient 432, an edge profile 433, and a thinner deposit area 434. Each region is characterized by distinct levels of grayscale intensity, corresponding to different material deposition requirements during the printing process.

The grayscale image 430A serves as a computational model to generate control commands for a 3D printhead, enabling precise deposition of a polymerizable mixture at specific locations. Darker regions, such as the thicker deposit 431, are associated with higher pixel intensity values and correspond to greater material deposition. Conversely, lighter regions, such as the thinner deposit 434, represent lower intensity values and correspond to reduced material deposition. The gradient region 432 and the edge profile 433 facilitate smooth transitions between areas of varying thickness, providing a realistic and functional replication of natural eye features, such as the iris and pupil.

In some embodiments, the control commands derived from the grayscale image 430A are executed over multiple printing passes, with each pass incrementally building up the required material volume. For example, the printhead may deposit a larger volume of polymerizable mixture in the thicker deposit region 431 during one pass and a smaller volume in the thinner deposit area 434 during subsequent passes. This layered deposition technique allows for the creation of a highly detailed and accurate prosthetic eye structure.

The edge profile 433 of the grayscale image 430A may particularly be important for achieving the natural curvature and boundary definition of the prosthetic eye. By varying the deposition intensity at the edges, the manufacturing system can create smooth, rounded transitions that replicate the natural contours of the eye. This feature may be important for providing both the aesthetic appeal and functional integration of the prosthetic eye.

In one example, the grayscale image 430A may be generated from a high-resolution scan of a user's functional eye, providing personalized replication of the iris pattern and other features. The scanned data is converted into a digital grayscale map, with pixel values representing the optical and physical properties of the original eye. This map is then used as the basis for generating control commands for the printing system.

In another embodiment, the grayscale image 430A may be dithered to enhance the smoothness and accuracy of the printed structure. Dithering algorithms, such as Floyd-Steinberg or Burkes, can be applied to distribute pixel intensity variations across the image, resulting in a more seamless deposition of the polymerizable mixture. This process minimizes artifacts and interstitial gaps in the final printed structure.

The upper surface gradient 432 depicted in the grayscale image 430A may be designed to mimic the natural depth and reflectivity of the iris. By varying the thickness and transparency of the deposited material, the printing system can create a multi-layered structure that interacts with light in a manner similar to a natural eye. For example, the gradient region 432 may be printed with semi-translucent polymerizable mixtures to achieve the desired optical effect.

The grayscale image 430A also supports the integration of additional functional features, such as hydration channels or electronic components. For example, specific regions within the thicker deposit 431 may be programmed to include micro-channels for oxygen permeability, enhancing the comfort and usability of the prosthetic eye. Similarly, conductive materials can be deposited in designated areas to enable electronic functionalities, such as dynamic pupil adjustment.

In some embodiments, the grayscale image 430A may be combined with other design maps, such as vein patterns or scleral features, to create a comprehensive model for printing the entire prosthetic eye. Each map provides specific instructions for material deposition, facilitating that all components are seamlessly integrated into the final product.

Following the deposition process, the polymerizable mixture may undergo a series of curing and pinning steps to stabilize and solidify the printed structure. For example, actinic radiation of specific wavelengths may be applied to partially polymerize the deposited material after each pass, allowing for subsequent layers to meld seamlessly with the underlying structure. A final curing process may involve exposure to higher-intensity radiation or heat, resulting in a fully solidified and functional prosthetic eye.

Referring now to FIG. 4F, the figure schematically illustrates an exemplary dynamic shape of a droplet of polymerizable mixture 441 at various time intervals 442 after being emitted from a printhead (not shown). Each contour within the figure represents a successive stage in the evolution of the droplet 441 as it travels through an ambient atmosphere toward a receiving substrate. The changing shapes depicted reflect the interplay of various forces acting upon the droplet 441, including gravitational pull, air resistance, surface tension, and the velocity imparted by the printhead during emission.

The droplet 441, as depicted at its initial state (time interval 1), emerges from the printhead as a substantially spherical entity due to the dominance of surface tension forces. As time progresses and the droplet 441 accelerates under the influence of the printhead's velocity, aerodynamic forces begin to deform the droplet into progressively elongated shapes, as shown by intervals 2 through 4. By the later stages, such as intervals 5 and 6, the droplet 441 may exhibit a flattened or even slightly distorted form, especially if subjected to turbulence or differential air pressures during its trajectory.

In embodiments of the present invention, the dynamic evolution of the droplet 441's shape may be accounted for when calibrating the additive manufacturing system. Specifically, the velocity, angle, and trajectory of the printhead are configured to align the droplet's final impact state on the receiving substrate with the desired deposition pattern. For example, during the manufacturing of a prosthetic eye, the polymerizable mixture droplets 441 are programmed to meld seamlessly with previously deposited layers, regardless of any transient shape deformations experienced during flight. The AI engine controlling the printhead can adapt deposition parameters in real-time to compensate for deviations in droplet shape or trajectory caused by external factors such as airflow fluctuations or variations in droplet volume.

The figure further highlights that the droplet 441's mass and viscosity play an important role in determining its trajectory and eventual deposition accuracy. Higher-viscosity polymerizable mixtures may retain a more uniform shape throughout their flight, making them particularly suitable for applications requiring precise layer definition, such as the creation of vein patterns or intricate iris structures within a prosthetic eye. Conversely, lower-viscosity mixtures may be leveraged for applications demanding broader, more diffuse deposition patterns, such as printing transparent outer layers.

In some embodiments, the printhead emits droplets 441 at varying sizes to achieve specific optical or structural effects in the prosthetic eye. For example, smaller droplets may be utilized to create fine details in the iris or pupil, while larger droplets are used for building bulk structures such as the scleral portion of the prosthetic eye. Each droplet's travel dynamics are precisely modeled and incorporated into the printing system's control algorithms, facilitating that even minute variations in droplet behavior do not compromise the final product's fidelity or functionality.

Additionally, FIG. 4F underscores the importance of pinning and curing processes in stabilizing the deposited droplets 441 upon contact with the substrate. Upon deposition, the droplet's dynamic energy dissipates, allowing it to spread slightly and meld with adjacent material. The system may employ actinic radiation, UV light, or thermal exposure to initiate gelation or pinning, preventing undesired movement or coalescence of the deposited material before subsequent layers are added.

In another aspect of the invention, the trajectory and deformation behavior of droplets 441 are leveraged to create complex geometries and textures on the prosthetic eye's surface. For example, by varying the emission angle and velocity of the printhead, the system can produce multi-directional deposition patterns, enabling the creation of naturalistic vein networks or light-reflective structures within the iris. This approach eliminates the need for post-processing or manual adjustments, significantly enhancing manufacturing efficiency and reducing production costs.

In some embodiments, the droplet shapes and trajectories depicted in FIG. 4F are also influenced by environmental conditions within the printing enclosure, such as temperature, humidity, and atmospheric pressure. The system may incorporate sensors to monitor these variables in real-time and dynamically adjust printhead operation to maintain consistent deposition quality. For example, higher humidity levels may increase air resistance and alter droplet trajectory, necessitating compensatory adjustments in printhead velocity or angle.

The figure also demonstrates the potential for integrating advanced modeling techniques, such as computational fluid dynamics (CFD), into the printing system's design and operation. By simulating the behavior of droplets 441 under various conditions, the system can preemptively optimize printhead configurations and deposition strategies, minimizing the need for iterative testing and manual recalibration.

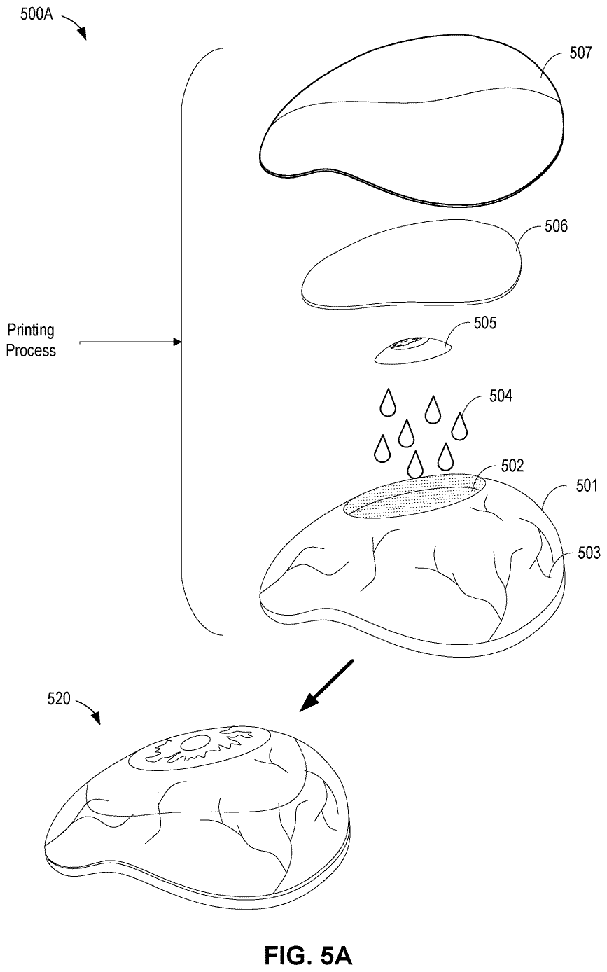

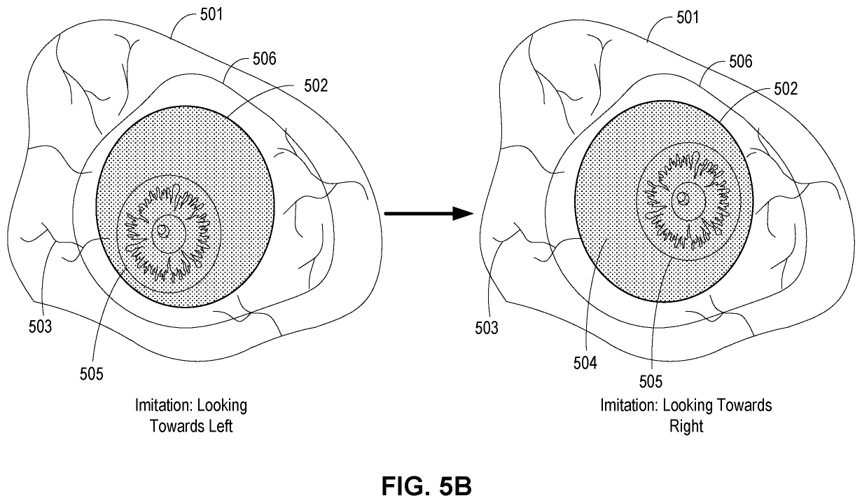

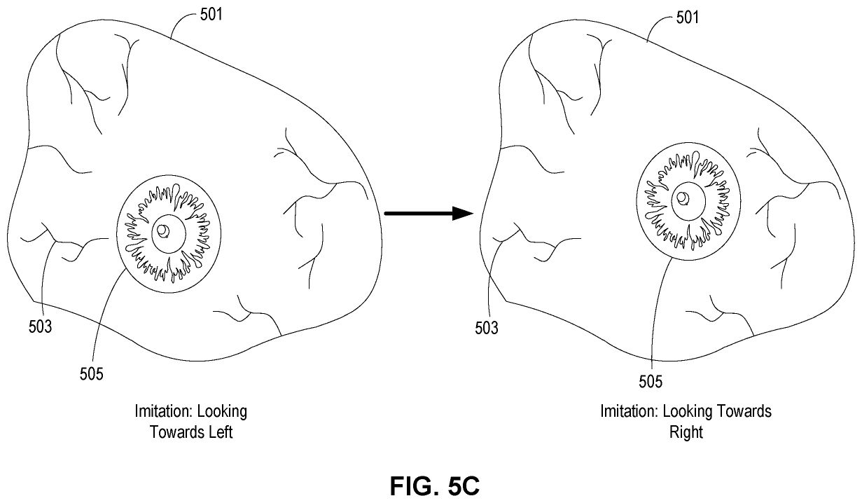

Referring now to FIG. 5A, an exemplary printing process 500A is illustrated for printing a prosthetic eye 520 with components mimicking the natural motion of the iris, according to some embodiments of the present invention. The printing process 500A begins with the additive manufacturing of a prosthetic base 501, which serves as the foundational structure of the prosthetic eye 520. The prosthetic base 501 may be printed layer by layer using patterned droplets, using one or more polymerizable mixtures that are selectively deposited and cured to form a solid structure. The cavity 502 may be formed during the printing process by strategically leaving a void space within the prosthetic base 501. This cavity 502 may be designed to house the iris 505 and allow controlled movement within the prosthetic eye 520. The formation of the cavity 502 may be controlled by adjusting the deposition pattern of the polymerizable material and utilizing temporary support structures that are removed after the cavity 502 is completed.

As the prosthetic base 501 is printed, vein patterns 503 may also be incorporated into its outer surface. These vein patterns 503 are designed to replicate the natural appearance of veins in a human eye. The vein patterns 503 may be printed using a different polymerizable mixture that contains pigmented or colored additives to match the aesthetics of the user's functional eye. The printing system used for this process 500A may include multiple printheads capable of depositing various polymerizable mixtures in different layers to achieve the desired vein pattern 503. The vein patterns 503 may also be printed in varying thicknesses to create a more realistic and three-dimensional appearance on the surface of the prosthetic base 501.

During the printing process 500A, a liquid 504 may be added to the cavity 502 to create a suspension medium for the iris 505. The liquid 504 may be injected into the cavity 502 through a precisely controlled dispensing mechanism. The liquid 504 may be selected to be transparent and to possess optical properties that enhance the visibility and movement of the iris 505 within the prosthetic eye 520. In some embodiments, the liquid 504 may be a saline solution, a biocompatible oil, or a specially formulated transparent gel that maintains stability and prevents evaporation over an extended period. The viscosity of the liquid 504 may be adjusted to allow the iris 505 to move naturally in response to external forces such as head movement and blinking.

Following the addition of the liquid 504, an iris 505 may be placed within the cavity 502. The iris 505 may be a 3D-printed component that is customized to match the user's functional eye. The iris 505 may be printed separately using an energy intensity pattern (e.g., 430 in FIG. 4D) derived from a scanned image of the user's functional eye, facilitating that details such as the iris texture, pupil size, and coloration are replicated accurately. Alternatively, the iris 505 may be manufactured using inkjet printing techniques to layer colored pigments onto a transparent or semi-transparent polymer substrate, giving it a natural depth and complexity.

The diameter of the iris 505 is intentionally smaller than the diameter of the cavity 502, allowing for slight movement within the cavity 502. This movement may be designed to mimic the natural motion of a human iris when the user changes their gaze direction. The liquid 504 acts as a suspension medium that facilitates the floating movement of the iris 505, reducing friction and enabling a smooth gliding motion within the prosthetic eye 520. The movement of the iris 505 may be influenced by gravitational forces, external accelerations from head movement, or magnetic interactions in some embodiments where additional control mechanisms are integrated.

Next, a transparent membrane 506 may be sealed over the cavity 502 to enclose the iris 505 and the liquid 504 securely within the prosthetic eye 520. The transparent membrane 506 serves multiple functions, including providing a smooth optical surface, maintaining the integrity of the liquid 504, and protecting the iris 505 from contamination or external damage. The transparent membrane 506 is slightly larger than the cavity 502 but smaller than the overall prosthetic base 501, allowing it to be seamlessly integrated into the prosthetic eye 520. The sealing process may involve a combination of UV-curable adhesive application and thermal bonding to achieve a permanent and leak-proof closure.

Following the sealing of the transparent membrane 506, one or more layers of polymerizable mixture 507 are deposited over the prosthetic base 501 and the transparent membrane 506. The purpose of these additional layers 507 is to refine the overall shape, enhance durability, and create a more natural-looking ocular surface. The deposited polymerizable mixture 507 may include a final clear coating that enhances the refractive properties of the prosthetic eye 520, allowing it to reflect and transmit light similarly to a natural cornea. This final layer also acts as a protective barrier that shields the internal components from environmental exposure and potential degradation.

To optimize the visual aesthetics and functional integration of the prosthetic eye 520, the outermost layers of polymerizable mixture 507 may be treated with finishing techniques such as microtexturing, polishing, and additional coloration. These techniques contribute to the overall realism of the prosthetic eye 520, making it visually indistinguishable from a natural eye when worn by the user. The final shape and curvature of the prosthetic eye 520 are designed to align with the user's ocular socket, providing a comfortable fit and natural appearance.

In some embodiments, additional functional enhancements may be integrated into the prosthetic eye 520. For example, embedded sensors may be included within the prosthetic base 501 to detect eye movement and adjust the position of the iris 505 accordingly. Additionally, magnetic components may be incorporated into the cavity 502 to control the movement of the iris 505 with external magnetic fields, allowing for fine-tuned adjustments that mimic pupil tracking.

The overall fabrication of the prosthetic eye 520 using the printing process 500A provides a high degree of customization and precision, enabling the production of prosthetic eyes that closely match the natural eyes of individual users. The ability to print intricate vein patterns 503, control the movement of the iris 505, and manipulate the optical properties of the prosthetic eye 520 through advanced layering techniques represents a significant advancement in prosthetic eye manufacturing.

The prosthetic eye 520 created through this method can be further modified based on user-specific requirements. For example, users with varying eye colors can have their prosthetic eye 520 precisely color-matched through pigment blending techniques incorporated into the polymerizable mixture 507. Likewise, for users requiring enhanced optical functionalities, specialized coatings may be applied to the outermost layers to reduce glare, improve contrast, or enhance UV protection.

The printing process 500A may be executed using a multi-step quality control system to verify the accuracy of each printed layer and provide the structural integrity of the final prosthetic eye 520. Optical scanners and high-resolution imaging systems may be employed to examine the printed prosthetic base 501, the integration of the vein patterns 503, the positioning of the iris 505, and the clarity of the transparent membrane 506. These quality control measures help maintain high manufacturing standards and improve the reliability of the prosthetic eye 520.

Referring now to FIGS. 5B-5C, an exemplary prosthetic eye system is illustrated, demonstrating the movement of an iris 505 within a cavity 502 of a prosthetic base 501, according to some embodiments of the present invention. The prosthetic base 501 comprises an internal cavity 502 in which the iris 505 is suspended, allowing it to shift within the cavity 502 in response to external stimuli or gravitational forces. A vein pattern 503 may be printed onto the prosthetic base 501 to mimic the natural appearance of an eye. The transparent membrane 506, which encloses the cavity 502, enables visibility of the iris 505 while securing the internal components.

FIG. 5B provides a detailed representation of the prosthetic eye system in a state where the iris 505 is suspended within a cavity 502 of the prosthetic base 501. The left-side diagram shows the iris 505 shifting towards the left, replicating a natural gaze imitating the user looking left, while the right-side diagram demonstrates the iris 505 moving towards the right, imitating a gaze to the right. The cavity 502 contains a liquid 504, which suspends the iris 505, facilitating its movement within the cavity.

The transparent nature of the cavity 502, the liquid 504, and the membrane 506 enclosing the cavity is highlighted for illustrative purposes. The liquid 504 is important for enabling the smooth motion of the iris 505, allowing it to move freely yet maintain its position relative to the user's head tilt or external stimulation. The movement is limited by the physical boundaries of the cavity 502, which has been meticulously designed to closely mimic the natural limits of iris motion within a human eye. The vein pattern 503 on the prosthetic base 501 provides an additional aesthetic layer, resembling the scleral veins of a natural eye.

The membrane 506, which seals the cavity 502, plays a dual function: it retains the liquid 504 and iris 505 securely within the cavity while allowing for the visual appearance of a seamless ocular surface. The transparency of the membrane 506 is carefully calibrated to avoid any optical distortion of the underlying iris 505 while providing structural integrity. This illustration, with its transparency, offers a comprehensive understanding of how the internal components of the prosthetic eye interact dynamically.

FIG. 5C presents a realistic visualization of the prosthetic eye in action, illustrating the movement of the iris 505 as it would appear in actual use, without the internal components such as the cavity 502, liquid 504, or the membrane 506 being visible. The design facilitates that the prosthetic eye looks indistinguishable from a natural eye, even during iris movement.

The left-side diagram demonstrates the prosthetic eye in a state where the iris 505 shifts towards the left, while the right-side diagram shows the iris 505 moving towards the right. Unlike FIG. 5B, this figure focuses on the external appearance of the prosthetic eye, emphasizing its lifelike quality. The iris 505, embedded within the prosthetic base 501, appears to move naturally, as though connected to the user's gaze direction. The vein pattern 503 on the prosthetic base 501 further enhances the realism, providing a lifelike scleral appearance.

The seamless movement of the iris 505 may be achieved through the dynamic interaction between the liquid 504 and the cavity 502. However, the encapsulating transparent membrane 506 is invisible, contributing to the illusion of a natural eye. The movement range of the iris 505 is precisely calibrated to mimic human ocular motion, limited by the cavity's dimensions while appearing fluid and unrestricted. FIG. 5C, therefore, highlights the end-user perspective of the prosthetic eye, demonstrating its ability to replicate natural eye movements convincingly.

By combining the technical transparency in FIG. 5B and the realistic view in FIG. 5C, these figures collectively provide a complete understanding of the prosthetic eye's functional and aesthetic capabilities. This design allows the prosthetic eye to seamlessly integrate into the user's facial appearance, providing a balance of realism, functionality, and adaptability.

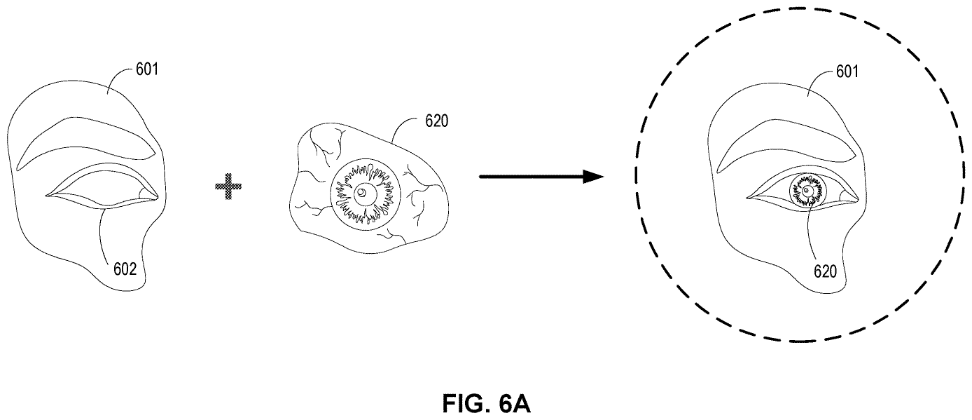

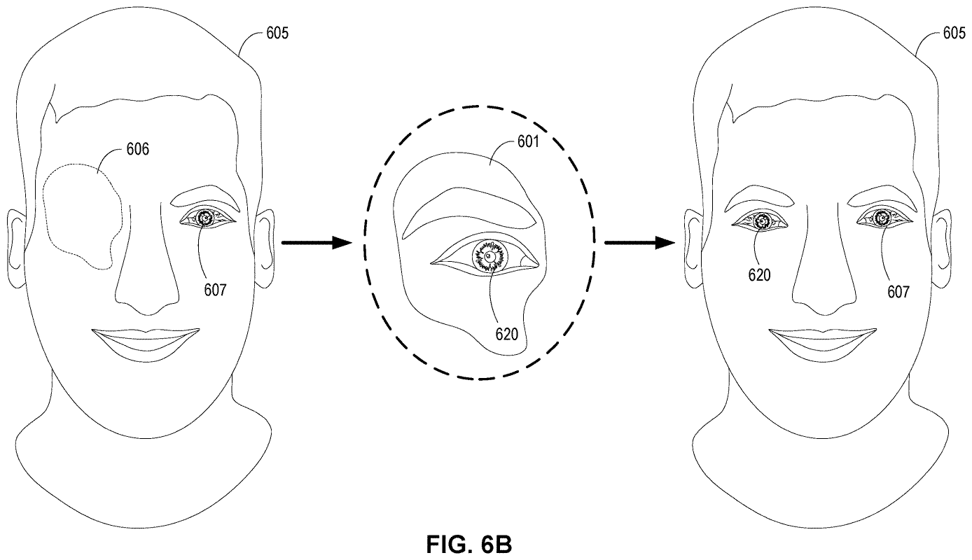

Referring now to FIGS. 6A-6B, an exemplary implant 601 with a prosthetic eye 620 is illustrated, wherein the implant 601 is structured to be attached to a damaged eye portion 606 of a user 605. According to some embodiments of the present invention, the implant 601 is designed to integrate a prosthetic eye 620 in a manner that restores a natural aesthetic appearance while also enabling functional movement to imitate the natural motion of an eye. FIG. 6A depicts an exploded view of the implant 601 and the prosthetic eye 620 being combined together, while FIG. 6B illustrates a broader perspective in which the implant 601, including the prosthetic eye 620, is affixed to the damaged eye portion 606 of the user 605.

The user 605 may have a damaged eye portion 606, which refers to a condition where the natural eye, including its surrounding soft tissues and structural support, has undergone trauma, disease-related degeneration, or surgical removal. In some cases, the damage may extend beyond the eyeball itself, affecting the eyelids, orbital bones, and connective tissue, making it difficult to accommodate traditional ocular prosthetics. The functional eye 607 remains intact, allowing for scanning and modeling processes that facilitate the customization of the prosthetic eye 620 to match the appearance of the functional eye 607.

The prosthetic eye 620 may be a 3D-printed prosthetic eye manufactured using an additive manufacturing technique as discussed in various embodiments of the present invention. The printing process may incorporate high-resolution scans of the functional eye 607 to replicate the shape, iris pattern, scleral details, and vein structures in the prosthetic eye 620, providing a natural and indistinguishable resemblance to the real eye. In some embodiments, the prosthetic eye 620 may be designed to include an artificial iris capable of limited movement within a cavity, as illustrated in FIGS. 5B-5C, allowing the prosthetic eye 620 to simulate gaze motion.

The implant 601 may include an eye socket 602, which serves as the receiving cavity for the prosthetic eye 620. The eye socket 602 may be shaped and dimensioned to securely hold the prosthetic eye 620 while allowing limited rotational or translational motion within predefined constraints. The eye socket 602 may be fabricated from a biocompatible material such as medical-grade silicone, polyethylene, or titanium-based composites, allowing for long-term integration with the user's orbital structure.

The attachment of the implant 601 to the damaged eye portion 606 may be performed using various methods depending on the severity of the damage and the structural requirements. In some embodiments, the implant 601 may be adhesively bonded to the remaining orbital tissue using medical-grade adhesives, providing a stable and secure fit. In other embodiments, the implant 601 may include embedded attachment points designed for integration with surgical anchors or titanium mesh implants that provide a rigid fixation to the orbital bones.

In cases where the surrounding orbital muscles are partially intact, the implant 601 may incorporate mechanical or electroactive elements that interact with residual muscle movements, allowing the prosthetic eye 620 to mimic limited natural motion. For example, the implant 601 may include a flexible membrane layer or micro-actuators embedded within the structure to respond to slight muscular contractions, enabling the prosthetic eye 620 to move in a synchronized manner with the user's functional eye 607.

The assembly process for the prosthetic eye 620 within the implant 601 may involve a stepwise approach wherein the prosthetic eye 620 is first positioned within the eye socket 602 and then secured using a retention mechanism. In some embodiments, the retention mechanism may include a magnetic attachment system, where small magnets embedded in the prosthetic eye 620 interact with corresponding magnetic points within the eye socket 602, enabling both secure placement and controlled movement.

Furthermore, the prosthetic eye 620 may incorporate additional layers or coatings that enhance durability and comfort. A final layer of polymerizable mixture may be deposited over the prosthetic eye 620, creating a protective and aesthetic finish that provides a glossy, life-like appearance similar to that of a natural eye. This outermost layer may also function as a hydrophilic surface, reducing friction and improving comfort for long-term wear.

In some embodiments, the prosthetic eye 620 may include embedded microfluidic channels or gel-like materials that mimic the tear film distribution found in a natural eye. These features may enhance the visual realism of the prosthetic eye 620, preventing excessive dryness and irritation while also maintaining an optimal surface texture.

The customization of the implant 601 may be further enhanced through digital modeling and AI-based design processes. By leveraging AI algorithms, the system can analyze the user's facial structure, functional eye 607 characteristics, and residual muscle functionality to generate an optimized design for both the implant 601 and the prosthetic eye 620. This customization process may also include the selection of vein patterns, scleral coloration, and iris textures that match the user's natural appearance.

Additionally, the prosthetic eye 620 may incorporate augmented reality or sensor-based technology for advanced functionalities. In some embodiments, a miniature camera or light sensor embedded within the prosthetic eye 620 may provide visual feedback or allow users to detect light intensity changes, enhancing spatial awareness for individuals with severe visual impairments.

The process of affixing the implant 601 and the prosthetic eye 620 to the user 605 may involve pre-surgical preparation, during which a digital scan of the damaged eye portion 606 is conducted to create a precise model for fitting. This process facilitates that the final assembly seamlessly conforms to the user's anatomical structure, eliminating discomfort and promoting long-term usability.

In cases where additional stabilization is required, the implant 601 may be designed with modular components that allow for post-surgical adjustments. For example, small adjustment screws or removable inserts may be incorporated within the implant 601, enabling minor refinements to the positioning and alignment of the prosthetic eye 620 over time.

Post-implantation care for the user 605 may include periodic maintenance and inspections to assess the integrity and functionality of the prosthetic eye 620. The prosthetic eye 620 may be removable, allowing the user to clean or replace specific components as needed. In some embodiments, a self-cleaning feature may be integrated into the prosthetic eye 620, utilizing microfluidic channels to distribute a cleaning solution across the surface, reducing the buildup of debris or irritation.

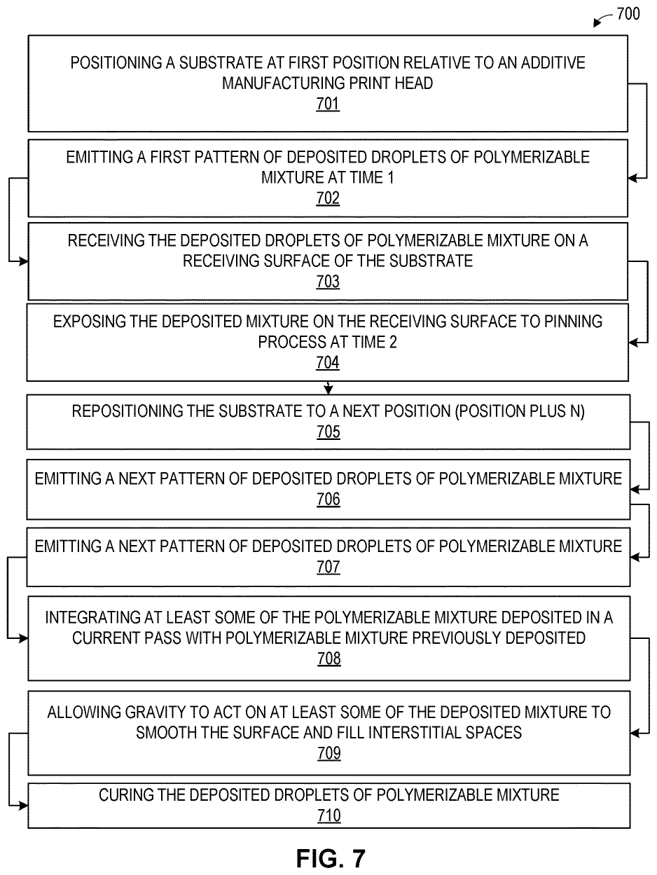

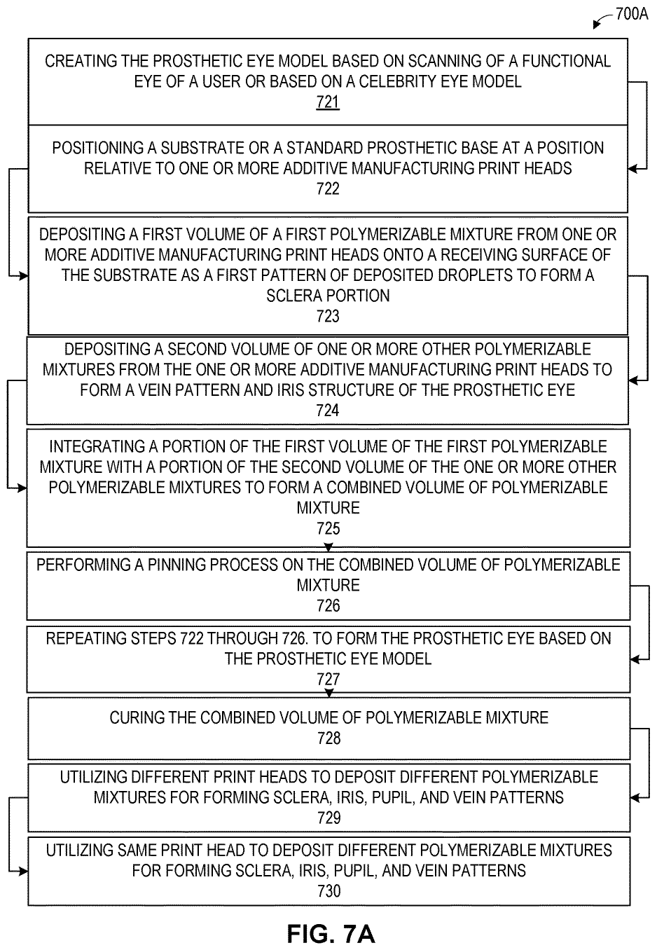

Referring now to FIG. 7, method steps 700 illustrate an exemplary sequence of operations that may be executed while practicing some implementations of the present invention. The method 700 generally pertains to an additive manufacturing process for forming a prosthetic eye or an ophthalmic device by depositing a polymerizable mixture in a controlled manner. The steps involve precise positioning, emission, and deposition of a polymerizable mixture, followed by processing steps that allow the deposited material to form a stable, structured, and functional final product. Each step of the method 700 is designed to contribute to the accuracy, strength, and functional integrity of the printed prosthetic eye, facilitating that the layered material properly integrates, smooths out, and cures to form the intended design.

At step 701, a substrate is positioned at a first position relative to an additive manufacturing print head. This initial positioning step is necessary to define the starting point for the deposition process. The substrate serves as a foundational receiving surface upon which the polymerizable mixture will be deposited in subsequent steps. The positioning process may involve precision-controlled movements facilitated by a robotic arm or a mechanical stage that adjusts the location of the substrate to match predefined coordinates. The positioning may also be guided by computer-aided design (CAD) data to align with the intended dimensions of the prosthetic eye being printed.

At step 702, the print head emits a first pattern of deposited droplets of polymerizable mixture at time 1. The print head may be configured to dispense droplets in a defined sequence, with parameters such as droplet size, velocity, and spacing precisely controlled. The emitted pattern may correspond to a specific layer of the prosthetic eye, facilitating that the base structure is gradually built up through successive depositions. The composition of the polymerizable mixture may include monomers, oligomers, photoinitiators, and other functional additives that facilitate curing in subsequent steps.

At step 703, the deposited droplets of polymerizable mixture are received on a receiving surface of the substrate. The receiving surface may be a pre-treated material designed to enhance adhesion while allowing for subsequent detachment of the final printed object. As the droplets make contact with the substrate, they begin to spread and partially integrate with any previously deposited layers. The surface properties of the substrate, such as temperature, texture, and wettability, may be optimized to facilitate uniform spreading and prevent undesired defects.

At step 704, the deposited polymerizable mixture is exposed to a pinning process at time 2. The pinning process may be performed to stabilize the deposited material before additional layers are applied. This process may involve exposure to actinic radiation, such as ultraviolet (UV) light, visible light, or other energy sources that initiate partial polymerization of the deposited material. Pinning helps in preventing excessive spreading of the polymerizable mixture, maintaining the structural integrity of the deposited pattern. The level of pinning may be controlled to allow subsequent layers to bond effectively with the partially cured material.

At step 705, the substrate may be repositioned to a next position, identified as position plus N. This movement prepares the printing system for the next deposition sequence. The repositioning may be performed in a linear or rotational manner, depending on the design and shape of the prosthetic eye being fabricated. In some embodiments, the repositioning may involve adjustments to the print head itself rather than moving the substrate. The system may rely on high-precision positioning mechanisms, such as stepper motors or piezoelectric actuators, to achieve accurate alignment.

At step 706, the print head emits a next pattern of deposited droplets of polymerizable mixture. This step involves the application of an additional layer of polymerizable material, following the same controlled deposition principles used in the previous emission step. The pattern may vary depending on the layer being printed, with different droplet arrangements contributing to the overall shape and function of the prosthetic eye. Layer thickness, droplet overlap, and deposition speed may be dynamically adjusted to accommodate complex geometries.

At step 707, another pattern of polymerizable mixture droplets may be emitted, continuing the layer-by-layer build-up process. This iterative process allows for precise shaping of internal structures, including cavities that may house movable components, liquid pouches, or embedded electronics. The material composition may also be modified between layers to introduce varying mechanical properties, such as stiffness or flexibility, where needed.

At step 708, the deposited polymerizable mixture from the current pass may be integrated with the previously deposited material. Integration may occur at a molecular level, with polymer chains from successive layers interpenetrating to form a cohesive structure. Thermal or chemical interactions between layers help in achieving strong interlayer adhesion, preventing delamination. This integration step may particularly be important for providing the structural durability of the prosthetic eye, as weak bonding may lead to material failure during use.

At step 709, gravity acts on the deposited mixture to smooth the surface and fill interstitial spaces. Natural forces such as surface tension and capillary action contribute to the self-leveling of the polymerizable material. This passive smoothing process helps in achieving a uniform surface texture without requiring additional mechanical intervention. In some embodiments, controlled vibrations or temperature modulation may be applied to further enhance material flow and eliminate inconsistencies.

At step 710, the deposited polymerizable mixture is cured to complete the printing process. Curing may involve a final exposure to actinic radiation, which triggers full polymerization of the material. The curing process may be performed in stages, with different wavelengths or intensities of light used to achieve optimal results. Post-curing treatments, such as annealing or chemical stabilization, may also be employed to enhance material properties. Once curing is complete, the printed prosthetic eye is ready for additional finishing steps, such as polishing, coloring, or the integration of additional functional components.

Referring now to FIG. 7A, a series of additional method steps 700A are illustrated for forming a prosthetic eye through an additive manufacturing process. The method involves multiple precise steps designed to replicate the appearance and function of a natural human eye while allowing for customization based on user-specific or celebrity-based eye models. The process begins with generating a prosthetic eye model using digital scans, followed by the strategic deposition of polymerizable mixtures that construct different anatomical features, including the sclera, vein patterns, and iris structure. Each stage in this process is carefully executed to facilitate that the deposited materials integrate properly, allowing for a lifelike prosthetic eye that closely resembles the natural human eye.

At step 721, the prosthetic eye model may be created based on scanning of a functional eye of a user or based on a celebrity eye model. The scanning process may involve high-resolution optical imaging, laser scanning, or structured light scanning to capture intricate details of the user's eye, including the shape of the sclera, the specific vein network, and the iris structure. The collected scan data is processed through advanced computational algorithms to generate a 3D digital model that serves as a blueprint for printing the prosthetic eye. In cases where the user selects a celebrity eye model, digital eye images from publicly available sources are processed to extract anatomical details. The model may be enhanced with additional textures and shading variations to improve the final printed output. The generated prosthetic eye model is then optimized for additive manufacturing, facilitating that the print head deposition parameters align with the structural and aesthetic requirements of the final prosthetic eye.

At step 722, a substrate or a standard prosthetic base may be positioned at a defined location relative to one or more additive manufacturing print heads. The positioning of the substrate is an important factor, as it directly influences the accuracy of the deposition process. In some embodiments, the substrate may be a pre-manufactured standard prosthetic base selected from a set of available sizes and shapes, allowing for customization at later stages of printing. The positioning mechanism may involve robotic arms, adjustable clamps, or a conveyor belt system to hold the substrate in place while the print heads operate. The substrate may also be pre-treated with adhesion-promoting coatings to facilitate the bonding of the deposited polymerizable mixtures. Once secured, the substrate undergoes an alignment check to verify its position relative to the print heads, preventing deviations that may lead to inaccuracies in the printed eye structure.

At step 723, a first volume of a first polymerizable mixture may be deposited from one or more additive manufacturing print heads onto a receiving surface of the substrate as a first pattern of deposited droplets to form a sclera portion of the prosthetic eye. The first polymerizable mixture may be specifically engineered to replicate the appearance of a natural human sclera, incorporating micro-pigments or nanoparticles that create the appropriate opacity, reflectance, and coloration. The print heads operate with high precision, depositing droplets in a controlled manner to achieve smooth layering and even distribution. In some embodiments, a transparent polymerizable mixture may be intermittently deposited along with the first polymerizable mixture to introduce subtle variations in translucency, mimicking the layered structure of a real sclera. The deposition process may be executed with real-time monitoring to assess droplet placement, volume consistency, and layer integrity, facilitating that the sclera portion of the prosthetic eye develops correctly.

At step 724, a second volume of one or more other polymerizable mixtures may be deposited from the one or more additive manufacturing print heads onto the receiving surface of the substrate to form a vein pattern and iris structure of the prosthetic eye. The second polymerizable mixture may contain CMYK pigments, allowing precise color blending to replicate the complex vascular structures of a human eye. The vein pattern may be generated using a predefined droplet arrangement, facilitating that the printed veins follow an organic, non-uniform path resembling natural blood vessels. Additionally, the iris structure may be printed using a separate droplet arrangement, incorporating radial fiber details that simulate the natural pigmentation and depth of a human iris. In some implementations, refractive index-modifying additives are incorporated into the second polymerizable mixture to enhance light scattering properties, improving the natural appearance of the printed iris. The deposition of the vein pattern and iris structure may be conducted in multiple passes, allowing for intricate layering that contributes to the depth and realism of the prosthetic eye.

At step 725, a portion of the first volume of the first polymerizable mixture may be integrated with a portion of the second volume of the one or more other polymerizable mixtures to form a combined volume of polymerizable mixture. The integration of these materials is a controlled process, where material intermixing occurs at precise junctions to create a seamless transition between the sclera, vein pattern, and iris structure. This step may involve controlled heating or localized exposure to actinic radiation to partially polymerize the combined mixture, promoting adhesion between the layers while maintaining the ability to deposit additional material in subsequent steps. The integration process may also involve adjusting deposition parameters such as droplet size, material flow rates, and curing intensities to facilitate that the combined volume develops in a structurally sound and visually accurate manner. In certain embodiments, a transparent polymerizable mixture may be intermittently introduced during this step to enhance three-dimensional effects, giving the prosthetic eye a more dynamic and lifelike appearance.

At step 726, a pinning process is performed on the combined volume of polymerizable mixture to achieve partial polymerization of the deposited materials. The pinning process involves controlled exposure to actinic radiation, such as ultraviolet (UV) or visible light, to initiate cross-linking of the polymer chains within the mixture without fully curing it. The purpose of this partial polymerization is to stabilize the deposited layers while maintaining sufficient flexibility for additional layers to be integrated. The pinning process may be executed using a calibrated exposure system that adjusts light intensity, wavelength, and duration based on the specific composition of the polymerizable mixtures. The localized polymerization prevents droplet diffusion and maintains high-resolution detailing in the vein patterns, iris structure, and sclera formation. The process also facilitates seamless integration of subsequent material layers, facilitating that each deposited droplet adheres securely without unintended spread or deformation.

At step 727, steps 722 through 726 are repeated iteratively to progressively build the prosthetic eye based on the prosthetic eye model. This iterative deposition facilitates that all anatomical components, including the sclera, vein network, iris, and pupil, are constructed with precision. Each repetition of the process refines the detail of the printed structures, allowing for greater depth, enhanced coloration, and improved textural fidelity. During each iteration, real-time monitoring systems may be employed to assess deposition accuracy, layer thickness, and any potential defects that require correction. The repetition process also allows for incremental modifications in material composition, where different polymerizable mixtures with varying viscosities, pigmentation, and refractive indices may be introduced to optimize the final prosthetic eye's appearance. In some embodiments, intermittent deposition of a transparent polymerizable mixture may be carried out to create subtle refractive effects that enhance the three-dimensional realism of the printed iris and sclera.

At step 728, the combined volume of polymerizable mixture is fully cured to finalize the structural integrity of the prosthetic eye. Curing involves comprehensive exposure to actinic radiation, heat, or other curing agents to solidify all deposited layers into a cohesive structure. The curing process eliminates any residual fluidity in the polymerizable mixtures, facilitating that all printed components retain their intended shapes and optical properties. The curing process parameters are carefully controlled to prevent excessive shrinkage or warping, which may compromise the anatomical accuracy of the prosthetic eye. In some embodiments, post-curing treatments may be applied, such as controlled thermal annealing or chemical stabilization, to enhance the mechanical strength and long-term durability of the prosthetic eye. Additionally, the curing phase allows for final adjustments in coloration and translucency, as specific polymerizable formulations may undergo slight spectral shifts upon complete polymerization.

At step 729, different print heads are utilized to deposit different polymerizable mixtures for forming the sclera, iris, pupil, and vein patterns. The use of multiple print heads enables precise control over the deposition of distinct materials, facilitating that each anatomical feature receives its appropriate formulation. A dedicated print head may be assigned for depositing the sclera portion, using a polymerizable mixture with embedded micro-pigments that match the natural scleral tone. Another print head may be configured to deposit a polymerizable mixture containing CMYK pigments for the vein patterns, allowing precise replication of vascular structures with realistic color variations. A separate print head may handle the iris structure, incorporating refractive index-modifying additives to enhance the optical depth of the printed iris. The pupil region may be printed using yet another print head, which deposits a high-opacity polymerizable mixture that effectively blocks light, so that the pupil appears deep and well-defined. The coordinated operation of multiple print heads optimizes the overall quality of the prosthetic eye, minimizing potential errors associated with single-head deposition.

At step 730, instead of utilizing multiple print heads, the same print head may be employed to deposit different polymerizable mixtures for forming the sclera, iris, pupil, and vein patterns. In this approach, a single print head is dynamically controlled to switch between different polymerizable mixtures, enabling sequential deposition of various anatomical features. This methodology is advantageous in scenarios where precise nozzle calibration is required to achieve seamless transitions between different material compositions. The print head may feature an integrated mixing chamber, allowing controlled blending of polymerizable mixtures before deposition to produce gradient effects, particularly in the iris region. The ability to utilize a single print head for multiple material depositions reduces system complexity and minimizes alignment errors that may arise from multiple print head configurations. In some embodiments, an adaptive printing algorithm is employed to modulate droplet ejection parameters based on real-time feedback from an imaging system, facilitating that each anatomical feature is constructed with the highest fidelity.

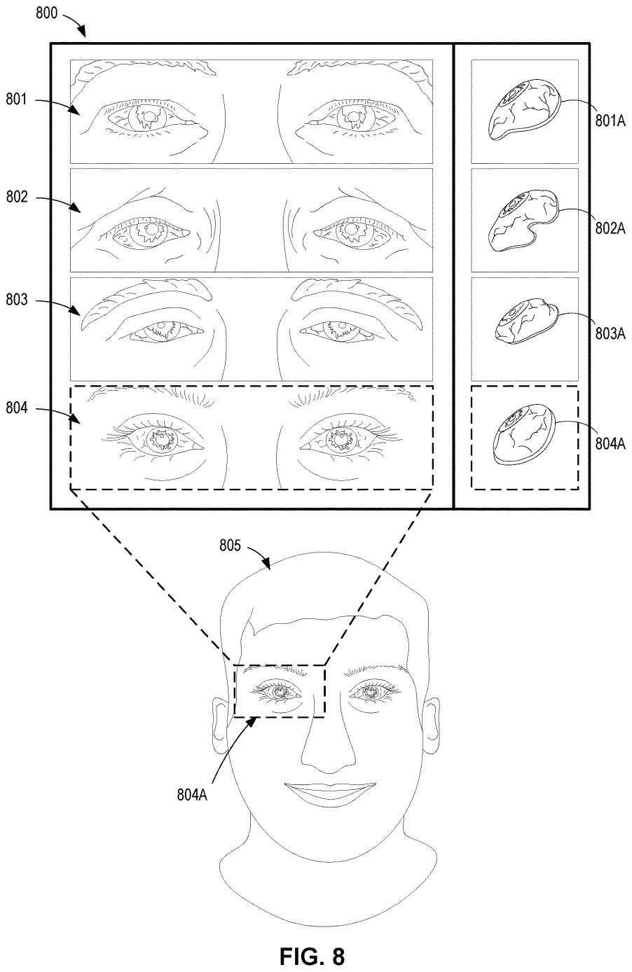

Referring now to FIG. 8, an exemplary system 800 is illustrated for generating prosthetic eyes based on eye samples 801-804 collected from various celebrities, public figures, or movie characters, with respective 3D-printed prosthetic models 801A-804A being fabricated for selection and use by a user 805, according to some embodiments of the present invention. The eye samples 801-804 may be acquired through multiple sources, including direct high-resolution scans from individuals who voluntarily participate in the process or through publicly available images and videos. Advanced image processing and artificial intelligence techniques may be utilized to reconstruct an accurate 3D model of the eye, including the shape, curvature, iris patterns, vein structures, and scleral details, providing a realistic replication of the selected eye.

In one embodiment, the eye samples 801-804 may be obtained by scanning celebrities' eyes during live events, professional photoshoots, or medical imaging, wherein a biometric scanner may generate detailed structural and colorimetric data of their eyes. Alternatively, for deceased or unavailable individuals, the reconstruction process may involve analyzing archived photographs and videos, where machine learning algorithms are used to interpolate missing features, enhancing the prosthetic's realism and depth perception. The process of 3D modeling and digital reconstruction allows for precise scaling and alignment, facilitating that the prosthetic eyes 801A-804A match the user's anatomical structure.

The reconstructed eye models 801-804 are then fed into an additive manufacturing system, such as those illustrated in FIGS. 2A-2C, where an advanced printing system (e.g., 200, 200A, 200B) fabricates the prosthetic eyes 801A-804A using multi-material polymerizable mixtures. The printing process may involve successive deposition of photopolymer resins to form the scleral portion, followed by the layered formation of the iris region with high-resolution pigment accuracy to match the scanned eye data. The process may also include printing microscopic vein-like structures in the sclera, thereby improving the natural appearance of the prosthetic.

Each prosthetic model 801A-804A may be designed to be customizable in terms of size, color tone, and shape, providing a seamless fit with the user's facial anatomy. Once the prosthetic models 801A-804A are fabricated, a user 805 may be presented with multiple options, allowing them to select a prosthetic that best suits their aesthetic preferences. For example, prosthetic eye 804A may be selected by user 805 based on a preference for a specific celebrity's eye design, or based on compatibility with the natural eye 607 for maintaining a symmetrical facial appearance.

In an alternative embodiment, the selection process of prosthetic eyes 801A-804A may involve augmented reality (AR) or virtual reality (VR) simulations, where a digital interface allows the user 805 to visualize how different prosthetic options would appear on their face before finalizing the selection. This may involve real-time facial mapping and depth-based 3D projection to align the prosthetic eye 804A within the user's ocular socket, facilitating that the prosthetic blends naturally with surrounding tissues.

Furthermore, in some embodiments, the prosthetic eyes 801A-804A may be designed to mimic natural eye movement, utilizing fluid-filled cavities, magnetically controlled iris structures, or miniaturized actuators to facilitate movement synchronization with the functional eye 607. This may enable the prosthetic eye 804A to replicate blinking, pupil dilation, or gaze shifting, enhancing the realism of the prosthetic.

In yet another embodiment, the prosthetic selection system 800 may be integrated with artificial intelligence-driven recommendations, where a deep-learning model analyzes the user's facial structure, skin tone, and existing eye features, suggesting the most aesthetically suitable prosthetic eye from the database of available models 801A-804A. The AI system may also allow users to adjust various parameters, such as iris intensity, scleral vein density, and pupil dilation range, customizing the final prosthetic eye 804A to their specifications.

After the user 805 has selected the desired prosthetic, additional post-processing steps may be carried out, such as UV curing, hydration treatment, and surface texturing, to enhance the durability and biocompatibility of the prosthetic. The finalized prosthetic eye 804A is then fitted onto the user 805, either as a removable ocular prosthetic or as a permanently affixed implant, depending on the nature of the eye condition.

In an advanced implementation, the prosthetic eye fitting process may involve biometric calibration, where the selected prosthetic eye 804A is mapped to the user's neuromuscular signals, enabling subtle movement coordination with the healthy eye 607. This process may be particularly useful for patients with partial ocular mobility, as the system can leverage existing neural feedback mechanisms to create an enhanced, lifelike motion experience.

Additionally, some embodiments may involve smart prosthetic enhancements, where the selected prosthetic eye 804A integrates microelectronics and embedded sensors, enabling vision augmentation for users with partial blindness. In such cases, the prosthetic may be equipped with low-light vision processing, object detection systems, or AR-based visual overlays, providing real-time enhancements to the user's perception of their surroundings.

Furthermore, the prosthetic eye 804A may be remotely adjusted after implantation using a mobile application or AI-based tuning system, where real-time customization of pupil dilation, color correction, and gaze alignment is performed based on environmental lighting conditions or user preferences. This approach may also allow for scheduled adjustments, where the prosthetic system autonomously optimizes its visual characteristics over time.

The 3D printing approach described in FIG. 8 allows for mass customization, meaning that the same base printing technology used for 801A-804A may be further adapted to create custom prosthetic designs for various medical, aesthetic, and cosmetic applications. This may include custom prosthetic designs for cosplay, theatrical performances, or advanced facial reconstruction surgeries.

Moreover, the scalability of this approach makes it suitable for mass production of affordable and high-quality ocular prosthetics, eliminating the need for traditional manual prosthetic fabrication, which is both costly and time-consuming. The system 800 can be extended to include an automated prosthetic dispensing system, where users worldwide can submit their eye scans via telemedicine platforms, receive customized prosthetic recommendations, and have their selected prosthetic eye 804A delivered to them.

In another embodiment, prosthetic models 801A-804A may include a biometric security feature, where embedded iris recognition technology allows the prosthetic to function as a biometric authentication system, enabling users to unlock devices, verify identity in security systems, or even interact with smart home automation platforms using a gaze-based interface.

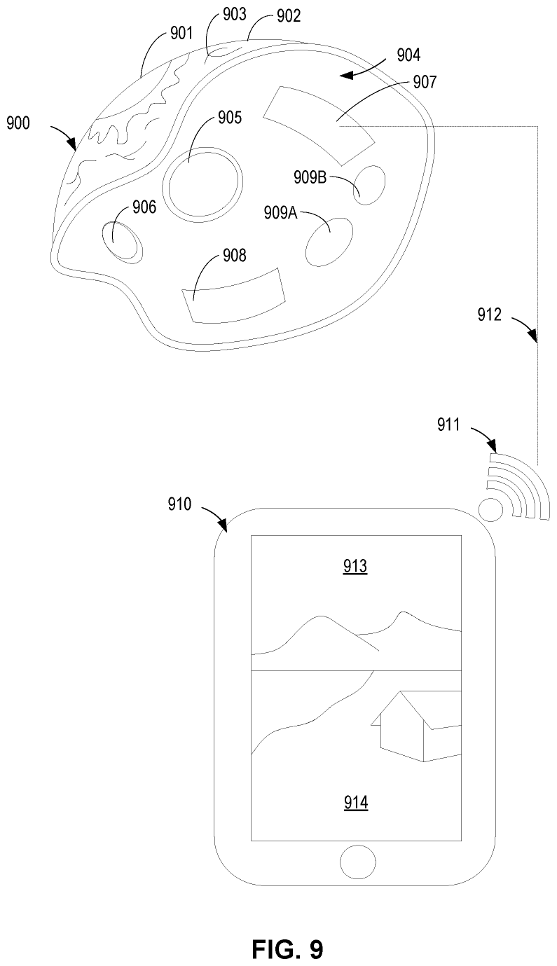

Referring now to FIG. 9, an exemplary prosthetic eye 900 is illustrated, incorporating advanced imaging, electronic circuitry, and sensor systems that enable real-time image capture, physiological monitoring, and wireless communication. The prosthetic eye 900 may be designed to not only replicate the appearance of a natural eye but also integrate functional elements that enhance usability for individuals with vision impairments or those requiring cosmetic prosthetic solutions. The prosthetic eye 900 comprises an iris portion 901 and a sclera 902, wherein the sclera 902 incorporates vein patterns 903, providing a realistic aesthetic appearance. The sclera 902, including the vein patterns 903, may be manufactured using an additive manufacturing system, employing layer-by-layer droplet deposition of polymerizable materials to form a structure that mimics the organic textures of a natural eye.

The prosthetic eye 900 may further include a plurality of integrated components designed to enhance functionality. A first camera 905 may be positioned towards the iris portion 901, enabling direct image capture from a frontal perspective, similar to the natural field of view of a biological eye. This first camera 905 can be an ultra-miniature imaging sensor capable of capturing high-resolution images and videos, which may be processed locally or transmitted to an external device for further analysis. The first camera 905 may employ autofocus mechanisms, optical image stabilization, and advanced processing algorithms to enhance clarity under varying lighting conditions.

A second camera 906 may be positioned at a lower portion 904 of the prosthetic eye 900, capturing a side perspective that is typically not visible through the primary iris-based viewpoint. The second camera 906 may be beneficial for providing peripheral vision or augmenting situational awareness for the user. This secondary imaging system may be equipped with a wide-angle lens or a fisheye lens, enabling a broader field of view and detecting objects approaching from non-frontal directions. Such a feature may particularly be advantageous for individuals engaged in activities where spatial awareness is important, such as navigating crowded environments or operating machinery.

The prosthetic eye 900 also incorporates an electronic circuitry 907, which may be fabricated on a flexible printed circuit board (PCB) and embedded within the scleral structure during or after the additive manufacturing process. The electronic circuitry 907 may include a microprocessor, memory storage, power regulation components, and wireless communication modules. The microprocessor may execute image processing tasks, data compression, and real-time analysis of captured visuals. The memory storage may be used for short-term buffering of image data before transmission or long-term storage for offline review.

The communication modules within the electronic circuitry 907 enable data transmission between the prosthetic eye 900 and external computing devices. Wireless communication protocols such as Bluetooth, Wi-Fi, or 5G connectivity may be integrated, allowing seamless interaction with mobile phones, smart glasses, or cloud-based servers. These modules facilitate live streaming of visual feeds, remote diagnostics, and software updates to enhance the prosthetic's capabilities over time. In certain embodiments, near-field communication (NFC) capabilities may be included to support short-range interactions with compatible medical devices.

The prosthetic eye 900 further comprises sensing elements 909A-909B, which function as physiological sensors to monitor various parameters related to ocular and systemic health. The sensing elements 909A-909B may include temperature sensors, pressure sensors, motion detectors, and biosensors capable of detecting biochemical markers in the tear film. Temperature sensors may monitor the thermal profile of the ocular surface, detecting abnormalities indicative of inflammation or infections. Pressure sensors may measure intraocular pressure, providing valuable data for individuals at risk of conditions such as glaucoma. Motion sensors may track eye movements, assisting in gaze-based interaction systems, while biosensors may detect glucose levels, hydration status, or infection markers in the tear film.

A battery 908 may be integrated within the prosthetic eye 900, providing power to the electronic components, imaging systems, and communication modules. The battery 908 may be a compact lithium-ion or solid-state battery optimized for prolonged usage with efficient power consumption. In some embodiments, the battery 908 may be rechargeable wirelessly through inductive charging or replaceable via a discreet compartment embedded within the prosthetic structure. Power management algorithms within the electronic circuitry 907 regulate energy distribution, optimizing battery longevity while maintaining functional performance.

In some implementations, the prosthetic eye 900 may include additional enhancement features such as artificial night vision, augmented reality overlays, or gesture-based control mechanisms. The integration of infrared sensors within the imaging modules may enable vision in low-light environments, providing adaptive visual support for users. Augmented reality overlays may be projected onto the captured visuals, enhancing navigation through real-time object recognition, textual annotations, or visual contrast adjustments. Gesture-based controls may involve blinking sequences or specific eye movements to trigger functions such as zooming, switching between camera views, or activating communication protocols.

The prosthetic eye 900 may also incorporate machine learning algorithms to analyze captured visual data, adapting to the user's environment and improving recognition accuracy. These algorithms may identify recurring patterns, detect faces, recognize text, or interpret gestures, enabling a more intuitive user experience. Data collected from the sensors and imaging modules may be processed in real time, allowing the prosthetic eye 900 to respond dynamically to environmental stimuli. For example, if the first camera 905 detects a sudden change in brightness, the electronic circuitry 907 may automatically adjust contrast and exposure settings to optimize visual clarity.

In some embodiments, the prosthetic eye 900 may be designed to synchronize with brain-machine interfaces (BMIs), allowing direct neural control of eye movements and functions. Electrodes embedded within the scleral structure may interface with neural pathways, enabling thought-controlled camera activation, zoom adjustments, or real-time visual augmentation. This feature may be particularly beneficial for individuals with neurological impairments, providing an intuitive means of interacting with their surroundings.

Furthermore, the prosthetic eye 900 may include adaptive biomimetic materials that respond to environmental changes, enhancing user comfort. These materials may adjust their hydration levels to maintain ocular moisture, reduce irritation, or change color to better match the user's natural eye under different lighting conditions. The incorporation of nanostructured coatings on the surface of the sclera 902 may also provide self-cleaning properties, repelling dust and microbial contaminants.

The design of the prosthetic eye 900 facilitates modular integration, allowing future upgrades or component replacements without requiring a complete re-manufacturing process. Individual modules, such as the cameras 905 and 906, battery 908, or sensors 909A-909B, may be configured as detachable units, allowing for easy maintenance and customization. This modular approach facilitates that advancements in imaging technology, communication protocols, or power management can be seamlessly incorporated into existing prosthetic models.

Additionally, the prosthetic eye 900 may be configured to interact with digital assistants or smart home systems, enabling hands-free control of connected devices. Voice commands or predefined eye movement gestures may be used to adjust lighting conditions, operate security cameras, or receive real-time notifications. Such integrations may enhance accessibility and independence for users with mobility impairments or visual disabilities.

In alternative embodiments, the prosthetic eye 900 may incorporate facial recognition capabilities to identify individuals and retrieve relevant information. This functionality may be useful in security applications, assisting individuals with memory impairments, or enabling personalized augmented reality experiences. Facial recognition data may be securely stored and processed within the electronic circuitry 907, with encryption measures implemented to protect user privacy.

The prosthetic eye 900 may also include emergency alert features, such as an integrated distress signal mechanism. In situations where the user requires immediate assistance, predefined gestures or voice commands may trigger an emergency notification sent to a designated contact or medical service. This feature may be particularly beneficial for elderly users, individuals with disabilities, or those in high-risk environments.

In some embodiments, the communication module embedded within the electronic circuitry 907 may be configured to establish a secure connection 912 with an external device such as a smartphone 910. The communication module may incorporate multiple wireless transmission protocols, such as Bluetooth, Wi-Fi, 5G, or near-field communication (NFC), allowing seamless interaction with modern mobile devices. The smartphone 910 may feature corresponding communication capabilities 911, which enable synchronization with the prosthetic eye 900, thereby facilitating real-time data exchange.

The smartphone 910, upon successful pairing with the prosthetic eye 900, may be utilized as a user interface for displaying visual data captured by the prosthetic eye 900. The first camera 905, oriented towards the iris portion 901, is capable of capturing a direct frontal view 913, providing real-time imagery of the scene directly in front of the user. The second camera 906, positioned at a lower portion 904 of the prosthetic eye 900, captures a side view 914, offering a perspective of objects or people approaching from an angle. These dual perspectives, displayed on the smartphone 910, provide the user or an assisting caretaker with comprehensive situational awareness.

In some embodiments, the smartphone 910 may also be configured to receive and display measurements obtained from the sensing elements 909A-909B integrated into the prosthetic eye 900. These sensing elements may include, but are not limited to, temperature sensors, pressure sensors, motion detectors, and biosensors. The smartphone 910 may process and visualize such data through a dedicated application, providing real-time physiological metrics such as ocular temperature, intraocular pressure, and hydration levels. This information may be beneficial for monitoring the user's ocular health, particularly for individuals susceptible to conditions such as dry eye syndrome, infections, or glaucoma.

The ability to relay real-time imaging and physiological data to the smartphone 910 may particularly be advantageous for individuals who are blind in one eye and rely on the prosthetic eye 900 for environmental perception. The smartphone 910, acting as an auxiliary visual aid, allows the user to obtain live feedback on their surroundings, enabling them to navigate more effectively. For example, the smartphone 910 may provide auditory cues based on image recognition algorithms, describing objects or individuals detected in the visual frame. Additionally, haptic feedback mechanisms may be employed, wherein vibrations or tactile signals are generated in response to detected obstacles, guiding the user through unfamiliar environments.

In another use case, if the user is completely blind in both eyes, the smartphone 910 may serve as an assistive tool for a caretaker. The caretaker can access live feeds from the prosthetic eye 900, gaining a real-time view of the user's surroundings through the displayed images 913 and 914. This functionality may be instrumental in guiding the user in daily activities, providing real-time assistance in mobility, social interactions, and safety monitoring. For example, the caretaker may observe whether the path ahead is clear or whether there are potential hazards in the user's environment.

In addition to live streaming of visual data, the smartphone 910 may include augmented reality (AR) features that enhance the usability of the prosthetic eye 900. AR overlays may be applied to highlight key objects, recognize faces, or provide contextual information about the surroundings. For example, the smartphone 910 may display object labels, street names, or directional arrows, offering enhanced spatial awareness for the user.

Furthermore, in some embodiments, the prosthetic eye 900 may incorporate voice recognition capabilities, allowing users to interact with the smartphone 910 using verbal commands. A built-in microphone may enable voice input, facilitating hands-free control of various functionalities. For example, the user may request the smartphone 910 to zoom in on a particular object, adjust contrast settings, or activate specific assistive features such as facial recognition or text-to-speech conversion.

The smartphone 910 may also serve as a storage device for recorded visual data, enabling users to review past footage captured by the prosthetic eye 900. This feature may be beneficial for security applications, personal documentation, or forensic analysis. Additionally, an AI-powered analytics engine within the smartphone 910 may analyze stored footage, identifying patterns or anomalies that may be relevant to the user.

Moreover, the smartphone 910 may include functionalities for remote monitoring and telemedicine. In such embodiments, the prosthetic eye 900 may periodically transmit health-related data to the smartphone 910, which can then relay the information to a healthcare provider. This system may allow medical professionals to track the user's ocular health remotely, providing proactive interventions when necessary.

In certain embodiments, the smartphone 910 may also function as a control panel for adjusting the prosthetic eye 900's settings. The user may modify brightness levels, toggle between different viewing modes, or activate specific camera functions directly from the smartphone 910. Additionally, the smartphone 910 may allow customization of the prosthetic eye 900's responses, enabling users to tailor settings according to their preferences and environmental conditions.

The integration of the smartphone 910 with the prosthetic eye 900 further enables data encryption and security features. Sensitive biometric data and visual recordings may be protected through encryption protocols, providing privacy and preventing unauthorized access. Multi-factor authentication mechanisms may be implemented to safeguard access to the stored data.

Additionally, the smartphone 910 may facilitate software updates for the prosthetic eye 900, enabling the deployment of firmware enhancements, bug fixes, and new features. Wireless update capabilities may allow continuous improvements in imaging algorithms, sensor calibration, and communication protocols, extending the lifespan and functionality of the prosthetic eye 900.

The communication link 912 between the prosthetic eye 900 and the smartphone 910 may also support emergency alert mechanisms. If the prosthetic eye 900 detects an emergency situation, such as a sudden fall, an automatic distress signal may be sent to a designated contact through the smartphone 910. This feature may be particularly useful for elderly users or individuals with mobility impairments.

In alternative embodiments, the smartphone 910 may be integrated with smart glasses, allowing the user to receive visual feedback through augmented reality displays. The smart glasses may wirelessly receive imaging data from the prosthetic eye 900, offering an immersive experience where digital overlays enhance real-world interactions.

Furthermore, in some implementations, the prosthetic eye 900 may synchronize with cloud-based services via the smartphone 910, enabling remote access to stored images and health metrics. Cloud synchronization may facilitate data sharing with medical professionals, enabling comprehensive diagnostics and long-term tracking of ocular health.

Additionally, the smartphone 910 may support multi-device connectivity, allowing integration with other assistive technologies such as hearing aids, smart home systems, or AI-powered personal assistants. This ecosystem of connected devices may enhance accessibility and convenience for users with disabilities.

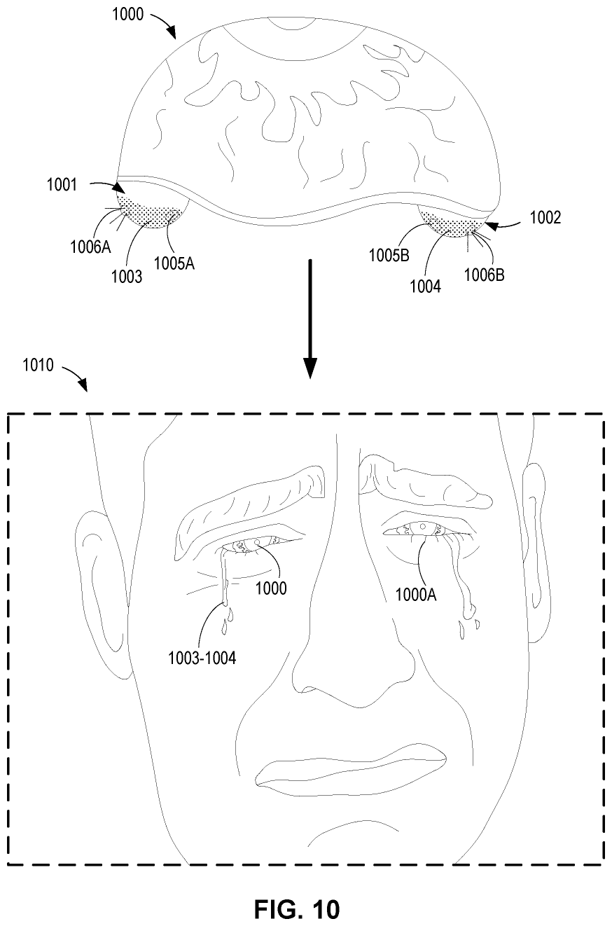

Referring now to FIG. 10, an exemplary prosthetic eye 1000 is illustrated comprising one or more liquid pouches 1001-1002 for medication delivery, hydration, and a weeping imitation, according to some embodiments of the present invention. The one or more liquid pouches 1001-1002 may be structurally integrated within the prosthetic eye 1000 during an additive manufacturing process, where the pouches are formed as part of the internal layers of the prosthetic eye 1000. Alternatively, the one or more liquid pouches 1001-1002 may be inserted after fabrication, allowing for modular replacement, maintenance, or refilling. The integration process may involve embedding flexible reservoirs in a designated compartment within the scleral structure of the prosthetic eye 1000, providing a seamless and natural appearance.

Each of the one or more liquid pouches 1001-1002 may feature one or more input ports 1005A-1005B, facilitating the controlled refilling of the pouches with a suitable liquid 1003-1004. The liquid used for refilling may be tailored for different functional aspects of the prosthetic eye 1000. For example, a first liquid 1003 may comprise a lubricating solution that hydrates the prosthetic eye 1000 and prevents dryness, providing prolonged wear comfort. A second liquid 1004 may include medicinal formulations, such as antibiotic or anti-inflammatory eye drops, intended for controlled therapeutic delivery over an extended duration. Alternatively, a third liquid composition may be introduced to simulate natural tear flow, creating a weeping effect when activated under specific conditions.

The prosthetic eye 1000 may be configured to store the same or different liquids in each of the liquid pouches 1001-1002 based on user preferences or medical recommendations. In some embodiments, the liquid pouches 1001-1002 may contain a multi-chamber system that segregates different liquids within a single pouch, allowing independent release based on user requirements. For example, a first chamber may hold an artificial tear solution for hydration, while a second chamber within the same pouch may hold a prescribed medication for treating ocular conditions.

The one or more liquid pouches 1001-1002 may be constructed using a flexible membrane that conforms to the curvature of the prosthetic eye 1000. The flexible membrane may be fabricated from biocompatible polymers, hydrogels, or silicone-based materials to provide durability and elasticity. The pouches may also feature micro-perforations or one or more orifices 1006A-1006B that regulate the release of liquid 1003-1004 in a controlled manner. The release mechanism may be designed to mimic the natural secretion of tears, dispensing liquid at a slow, steady rate to maintain hydration or deliver medication effectively.

In one embodiment, the release of liquid 1003-1004 may be activated through user-applied pressure on the prosthetic eye 1000. This activation mechanism may be designed such that gentle squeezing of the prosthetic eye 1000, for example, during blinking or intentional eye closure, induces a controlled discharge of liquid from the liquid pouches 1001-1002. The internal structure of the prosthetic eye 1000 may incorporate mechanical valves or pressure-sensitive membranes that regulate fluid flow only when a specific threshold of force is applied.

Another embodiment may involve an automatic activation mechanism wherein liquid 1003-1004 may be dispensed in response to natural biological triggers. For example, when the user exhibits emotional distress, such as crying, the prosthetic eye 1000 may replicate the release of liquid 1003-1004 to create a synchronized weeping effect. This may be achieved using embedded microfluidic channels that detect increased muscle contractions or tension around the eye socket, triggering a release from the liquid pouches 1001-1002.

As illustrated in FIG. 10, a user 1010 wearing the prosthetic eye 1000 is shown experiencing a weeping effect. The functional eye 1000A exhibits the natural flow of tears, while the prosthetic eye 1000 concurrently releases liquid 1003-1004, producing a visually coherent response. This synchronization enhances the realism of the prosthetic eye 1000, making it appear more lifelike in social interactions.

The ability to mimic natural tear flow in response to emotions may have psychological benefits for individuals wearing the prosthetic eye 1000. For example, users who have lost an eye due to trauma or medical conditions may find increased comfort in knowing that their prosthetic eye 1000 behaves similarly to their functional eye 1000A, reducing self-consciousness in social settings.

Beyond aesthetics, the controlled release of liquid 1003-1004 from the prosthetic eye 1000 may also serve medical applications. Patients suffering from dry eye syndrome, for example, may benefit from periodic hydration provided by the prosthetic eye 1000. In such cases, the liquid 1003 dispensed may include artificial tears or other ophthalmic solutions that replicate the natural lubrication process.

Similarly, individuals requiring continuous medication for ocular conditions such as glaucoma or infections may utilize the prosthetic eye 1000 for sustained drug delivery. The prosthetic eye 1000 may be programmed to release a precise dosage of medicated liquid 1004 at predetermined intervals, eliminating the need for manual eye drop application.

In another embodiment, the prosthetic eye 1000 may be equipped with a sensor-based activation mechanism that detects environmental factors such as humidity, temperature, or wind conditions. When the surrounding environment is excessively dry or windy, the prosthetic eye 1000 may autonomously release a moisturizing liquid 1003 to maintain comfort.

The input ports 1005A-1005B of the liquid pouches 1001-1002 may be designed for ease of access, allowing the user to refill or replace the liquid contents as needed. These ports may include secure sealing mechanisms to prevent leakage or contamination, such as twist-lock valves or silicone-sealed openings.

Additionally, the liquid pouches 1001-1002 may incorporate antimicrobial coatings to maintain hygiene and prevent bacterial growth. This may particularly be beneficial for users who require frequent medication delivery through the prosthetic eye 1000, as it minimizes the risk of infections.

The prosthetic eye 1000 may also include compatibility with smart monitoring devices that track fluid levels within the liquid pouches 1001-1002. A mobile application or external reader may notify the user when a refill is required, providing uninterrupted functionality of the prosthetic eye 1000.

In alternative embodiments, the prosthetic eye 1000 may feature replaceable liquid cartridges instead of refillable pouches. This modular approach allows users to swap out pre-filled cartridges containing different liquid formulations, providing flexibility in usage.

Moreover, the prosthetic eye 1000 may be designed with customizable liquid flow rates, enabling users to adjust the intensity of liquid release based on personal comfort preferences. This may be achieved through a manual dial or an electronically controlled micro-pump embedded within the structure of the prosthetic eye 1000.

The design of the prosthetic eye 1000 may also accommodate different shades of liquid for theatrical or artistic applications. For example, colored solutions may be used to create dramatic visual effects, such as replicating fictional characters with enhanced ocular aesthetics.

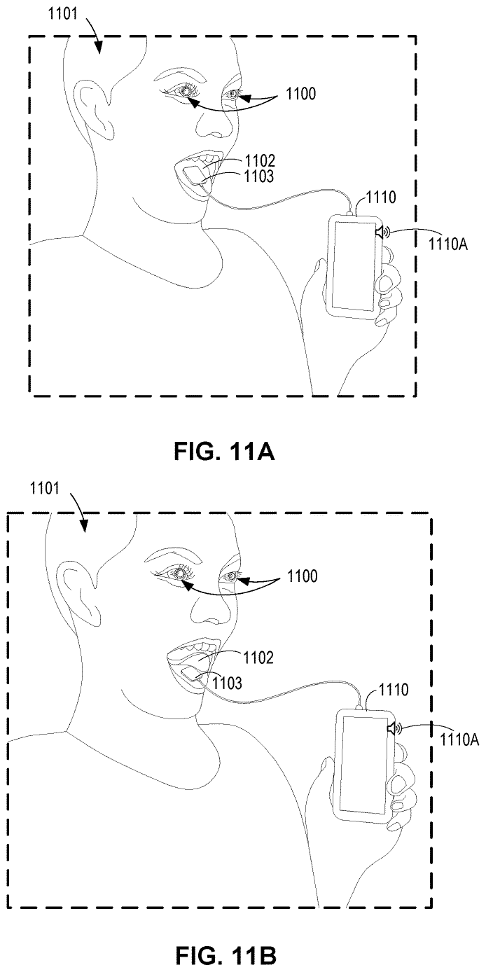

Referring now to FIGS. 11A-11B, an exemplary system is illustrated in which prosthetic eyes 1100 are coupled to an Electronic sensory device 1103, allowing a blind user 1101 to sense visual signals transmitted from the prosthetic eyes 1100. The system facilitates non-visual sensory perception of the surrounding environment by converting visual data into electro-tactile stimulation on the tongue 1102 of the user 1101, thereby enabling a novel method of spatial awareness for individuals who are visually impaired. The prosthetic eyes 1100 may integrate cameras, sensors, and electronic circuitry that acquire real-time visual information and transmit the processed signals to the Electronic sensory device 1103. The Electronic sensory device 1103 may then stimulate the tongue 1102 of the user 1101 with a specific electro-tactile pattern, which, over time, the user 1101 may learn to interpret as a representation of their environment.

The prosthetic eyes 1100 may incorporate one or more wireless communication modules to establish a connection with an external device 1110, which may be a smartphone, tablet, or a dedicated processing unit. The external device 1110 is configured to receive and process the video feed captured by the prosthetic eyes 1100, extract relevant spatial information, and convert the data into signals optimized for the Electronic sensory device 1103. The external device 1110 may include artificial intelligence-based image recognition, depth sensing, and motion detection algorithms to enhance the perception provided to the blind user 1101.

As illustrated in FIG. 11A, the Electronic sensory device 1103 is placed on the tongue 1102 of the user 1101, where the electro-tactile array stimulates different regions of the tongue 1102 based on the spatial distribution of objects detected by the prosthetic eyes 1100. The stimulation pattern may correspond to variations in brightness, object proximity, and depth, allowing the user 1101 to discern shapes and movement. In FIG. 11B, an alternative positioning of the Electronic sensory device 1103 is illustrated, where the device is placed below the tongue 1102, offering a different sensory experience while still providing spatial awareness through electro-tactile feedback.

The Electronic sensory device 1103 may be designed as a compact, wearable apparatus that may include an array of electrodes, a wireless communication module, a processing unit, and an adjustable power source. The electrodes are designed to deliver precise electrical impulses corresponding to visual data, creating an artificial perception of space through controlled stimulation of the tongue 1102. The Electronic sensory device 1103 may be powered through a rechargeable battery, which may be charged via a USB port or a wireless charging module integrated within the external device 1110.

The external device 1110 may incorporate various processing circuitries, including a graphics processing unit (GPU) for real-time image rendering, a neural processing unit (NPU) for AI-based scene interpretation, and a secure data transmission module for encrypted communication between the prosthetic eyes 1100 and the Electronic sensory device 1103. Additionally, the external device 1110 may support voice-assisted navigation, enabling the user 1101 to receive verbal descriptions of their surroundings in conjunction with the electro-tactile feedback.

The system may support multiple sensory modes, allowing the blind user 1101 to toggle between different levels of sensitivity, contrast detection, and object tracking. For example, a high-sensitivity mode may provide finer details of objects, while a motion detection mode may prioritize moving objects to assist with navigation in crowded environments. The Electronic sensory device 1103 may also be customized based on individual user preferences, with adaptive learning algorithms refining the electro-tactile feedback to match the cognitive processing capabilities of the user 1101.

In some embodiments, the prosthetic eyes 1100 may feature adjustable focal lengths and dynamic aperture control, enabling selective focus on objects at different distances. The processed images may be converted into a simplified spatial map that is translated into electro-tactile feedback patterns. The Electronic sensory device 1103 may then generate an intuitive sensory representation of the surrounding space, allowing the user 1101 to navigate unfamiliar environments with enhanced confidence.

The communication between the prosthetic eyes 1100, the Electronic sensory device 1103, and the external device 1110 may be achieved through Bluetooth, Wi-Fi, NFC, or other secure wireless transmission protocols, providing low-latency and high-speed data transfer for real-time sensory substitution. The external device 1110 may further allow caregivers or medical professionals to remotely access system parameters, adjust sensitivity settings, and provide real-time guidance to the user 1101.

The system described in FIGS. 11A-11B may be particularly beneficial for users who are blind in both eyes, as it provides a non-visual method of perceiving the environment. However, it may also be applicable to individuals with partial vision loss, allowing them to augment their remaining vision with electro-tactile feedback. In cases where a user 1101 has a functional eye in one socket and a prosthetic eye 1100 in the other, the system may operate in a hybrid mode, wherein the functional eye provides natural vision while the prosthetic eye 1100 enhances spatial awareness.

By integrating machine learning algorithms, the Electronic sensory device 1103 may evolve over time, adapting its electro-tactile patterns based on user experience and feedback. For example, the system may track eye movements, head orientation, and environmental conditions to optimize sensory stimulation. If the user 1101 is in a dimly lit environment, the external device 1110 may adjust brightness levels and enhance contrast before transmitting the processed image to the Electronic sensory device 1103.

In another embodiment, the Electronic sensory device 1103 may feature a multi-zone electro-tactile array, wherein different sections of the tongue 1102 receive specialized stimuli corresponding to different regions of the captured image. This multi-zone feedback system allows for a more detailed and immersive spatial perception experience. For example, objects appearing in the upper field of view may stimulate the upper portion of the tongue 1102, while objects in the lower field of view may be represented by tactile impulses in the lower tongue region.

Furthermore, the system may incorporate speech-to-tactile conversion, where textual information or spoken words detected by the prosthetic eyes 1100 may be translated into tactile vibrations on the tongue 1102, enabling blind users to interpret written signs or spoken dialogue through an electronic sensory device interface.

In some implementations, the prosthetic eyes 1100 may be equipped with thermal imaging capabilities, allowing the user 1101 to detect temperature variations in their surroundings. This feature may be particularly useful for identifying people, animals, or heated objects in dark environments, with the Electronic sensory device 1103 encoding temperature differences into varying intensities of electro-tactile stimulation.

By integrating biofeedback sensors, the system may also assess stress levels, heart rate, and pupil dilation, adjusting sensory feedback accordingly to provide a more comfortable experience for the user 1101. For example, if heightened stress levels are detected, the system may moderate stimulation intensity to prevent sensory overload.

The prosthetic eyes 1100 may additionally support gesture recognition, enabling the user 1101 to control specific features of the system through facial expressions or head movements. This hands-free interaction may enhance usability, particularly in situations where manual input is impractical.

In some implementations, the external device 1110 may incorporate an AI engine configured to process visual signals received from the prosthetic eye 1100 and generate real-time auditory feedback through an integrated speaker 1110A. The AI engine may execute advanced computer vision algorithms, deep learning models, and object recognition techniques to analyze the captured video stream, detect objects, classify them, and translate the detected information into spoken words or descriptive phrases. The speaker 1110A may then play these descriptions aloud, allowing the user 1101 to hear what is in front of him, thereby supplementing the tactile sensory feedback generated by the Electronic sensory device 1103.

In some embodiments, the AI engine in the external device 1110 may be designed to assist the user 1101 in learning how to interpret the sensory feedback from the Electronic sensory device 1103. The AI engine may generate descriptive audio cues for objects, distances, movement, and other environmental factors, providing the user with additional reference points that can be cross-matched with the electro-tactile stimulation received through the Electronic sensory device 1103. For example, when the prosthetic eye 1100 detects an obstacle, such as a tree, the AI engine may process the image, recognize the object as a tree, and play an audio message through the speaker 1110A, stating: “There is a tree in front of you.” At the same time, the Electronic sensory device 1103 may generate a corresponding pattern of electro-tactile feedback, reinforcing the sensory association between the tactile stimulus and the presence of an obstacle.

This combination of auditory and tactile feedback may be particularly beneficial for users who are initially unfamiliar with the Electronic sensory device 1103. Without the audio guidance, the user 1101 may struggle to interpret the tactile stimulation patterns, making it difficult to understand whether the sensory signals correspond to a wall, a chair, a staircase, or a moving object. However, by hearing an explanation of what the Electronic sensory device 1103 is detecting, the user 1101 may progressively learn to associate certain patterns with specific real-world objects. Over time, this learning process may allow the user 1101 to rely primarily on the Electronic sensory device 1103, with the AI-generated audio descriptions serving as an optional secondary aid.

In some embodiments, the AI engine in the external device 1110 may further include facial recognition capabilities, allowing the system to detect and identify individuals in the user 1101's field of view. By leveraging a database of stored facial features, the AI engine may analyze facial structures, match them with stored profiles, and announce the name of the detected person through the speaker 1110A. For example, when a friend named Adam is walking towards the user 1101, the speaker 1110A may play a voice message saying: “Your friend Adam is approaching you.” This real-time feedback may enable the user 1101 to recognize acquaintances, friends, family members, or colleagues without needing visual confirmation.

As the user 1101 continues to interact with the system, he may gradually internalize the sensory output patterns generated by the Electronic sensory device 1103 in response to different individuals. For example, after repeated encounters with Adam, the user 1101 may learn to recognize the specific sensory pattern associated with Adam's presence. Eventually, when the Electronic sensory device 1103 generates this sensory pattern, the user 1101 may instinctively recognize Adam's presence without requiring an audio announcement from the speaker 1110A. This learning process may be similar to how sighted individuals recognize familiar faces at a glance, allowing the user 1101 to develop a non-visual perception of the people around him.

In another example, the AI engine may recognize a moving vehicle and announce through the speaker 1110A: “A car is passing by on your left.” Simultaneously, the Electronic sensory device 1103 may generate a dynamic tactile stimulation pattern that corresponds to the movement of the car from left to right. Over time, the user 1101 may learn to associate this tactile pattern with the presence of moving vehicles, enabling him to navigate busy streets without relying entirely on auditory cues.

In some embodiments, the AI engine may also assist in navigation and wayfinding by analyzing the surrounding environment and providing directional cues through the speaker 1110A. For example, when the user 1101 approaches a staircase, the AI engine may generate a voice message stating: “There is a staircase ahead. Step up.” In a different scenario, if the user 1101 is looking for an exit, the AI engine may process the captured images and announce: “The exit is 10 meters ahead on your right.” This functionality may be particularly useful in complex indoor environments such as shopping malls, airports, train stations, and office buildings, where the user may need assistance in locating specific areas.

In some implementations, the AI engine may include scene analysis algorithms capable of detecting and describing entire environments rather than individual objects. For example, if the user 1101 is walking in a park, the speaker 1110A may play an ambient description such as: “You are in a park. There are trees around you, and a bench is 5 meters ahead.” This broader context may provide the user 1101 with a more immersive understanding of his surroundings, allowing him to navigate confidently without requiring constant detailed descriptions of every object.

Additionally, the AI engine may be programmed to detect and interpret gestures and expressions from people interacting with the user 1101. For example, if a person waves at the user 1101, the system may recognize the gesture and play an audio message through the speaker 1110A stating: “Someone is waving at you.” If a person smiles, the system may provide an audio cue such as: “The person in front of you is smiling.” This feature may be particularly beneficial for social interactions, allowing the user 1101 to interpret non-verbal communication cues that would otherwise be lost due to visual impairment.

In some embodiments, the AI engine may support customizable audio descriptions, enabling the user 1101 to adjust the level of detail provided by the speaker 1110A. For example, in a simplified mode, the AI engine may provide only basic object recognition, such as “chair,” “door,” or “person.” In an advanced mode, the AI engine may provide more detailed descriptions, such as “A red chair with armrests is in front of you,” or “A man wearing a blue shirt and glasses is standing 3 meters away.” This flexibility may allow users to tailor the system to their personal preferences and needs.

In another embodiment, the AI engine may integrate natural language processing (NLP) capabilities, allowing the user 1101 to interact with the system through voice commands. For example, the user 1101 may ask the system: “Is there anyone in front of me?”, and the AI engine may respond through the speaker 1110A: “Yes, your friend Sarah is standing 2 meters away.” Similarly, the user 1101 may ask: “Where is the nearest door?”, and the AI engine may analyze the captured images and reply: “The nearest door is on your right, 5 meters ahead.”

Furthermore, in some embodiments, the AI engine may be trained to detect potential hazards in real-time and provide immediate alerts. For example, if the user 1101 is approaching a low-hanging branch, the AI engine may issue an urgent warning: “Obstacle ahead. Duck down slightly.” Similarly, if the user 1101 is about to step into an open manhole, the system may generate an important alert: “Danger! Stop walking forward.” These real-time alerts may significantly enhance safety and mobility for the visually impaired.

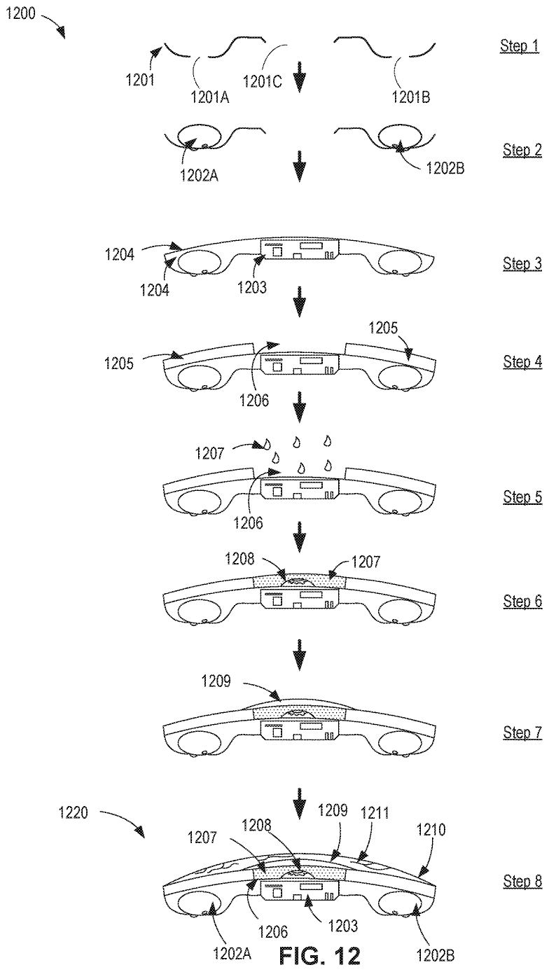

Referring now to FIG. 12, an exemplary sequence of manufacturing steps 1200 for fabricating a prosthetic eye 1220 with an internal cavity, liquid pouches, and integrated electronic circuitries is illustrated, according to some embodiments of the present invention. The prosthetic eye 1220 may be fabricated using an additive manufacturing system, which may include layer-by-layer droplet deposition of polymerizable mixtures to construct a functional prosthetic eye with embedded components. The additive manufacturing system may be configured to precisely deposit polymerizable materials to create predefined cavities within the structure of the prosthetic eye, which may later accommodate additional functional components such as liquid pouches for medication, hydration, and weeping effect, as well as electronic circuitry for sensory functions and communication interfaces. The fabrication process may involve multiple deposition cycles, with different materials being used at specific stages to impart distinct functional characteristics to the prosthetic eye. The manufacturing method 1200 may involve four primary steps, as described below.

At Step 1, the polymerizable mixture may be deposited in a layer-by-layer droplet deposition fashion to create an initial structure 1201 of the prosthetic eye 1220. This structure may include one or more cavities or ports 1201A, 1201B, and 1201C. These cavities are strategically positioned within the prosthetic eye to accommodate various functional elements. The cavities 1201A and 1201B may be designated for the insertion of liquid pouches that store and release fluids for medication delivery, hydration, or simulating natural tear secretion. The cavity 1201C may be intended for housing electronic circuitry 1203, which may include processing units, sensors, and communication modules. The additive manufacturing system may precisely define the geometries and depths of these cavities to accommodate their respective components while maintaining structural integrity.

In some implementations, the polymerizable material deposited at this stage may include biocompatible polymers with varying degrees of flexibility and transparency. The regions forming the sclera may be deposited with a more rigid polymer, while the regions forming the corneal and iris areas may be composed of a translucent polymerizable mixture. Additionally, the internal cavity walls may be designed with microscopic channels to allow the controlled flow of liquid from the liquid pouches toward the tear ducts, creating a weeping effect.

At Step 2, the liquid pouches 1202A and 1202B may be inserted into the cavities 1201A and 1201B, respectively. These pouches may be fabricated separately and inserted manually or automatically during the printing process. The liquid pouches may contain sterile solutions, lubricating agents, or controlled-release medications, which may be used for hydrating the prosthetic eye, delivering ocular drugs, or mimicking natural tear production.

The liquid pouches 1202A and 1202B may be composed of flexible, biocompatible membranes, such as silicone-based or hydrogel materials, that can expand and contract in response to external pressure. The pouches may include microvalves or porous membranes to regulate the flow of liquid and prevent uncontrolled leakage.

In some embodiments, hydrophilic polymers may be integrated into the cavity walls, allowing gradual absorption and release of moisture, further enhancing the comfort and hydration levels for the wearer. The liquid composition may also include electrolytes and artificial tear substitutes, particularly useful for individuals suffering from dry eye syndrome or ocular discomfort.

At Step 3, an electronic circuitry 1203 may be placed within the cavity 1201C. This circuitry may include one or more sensors, communication modules, power management circuits, and microcontrollers, enabling real-time data collection, communication with external devices, and automated control of liquid release. The electronic circuitry may be removable or replaceable, allowing maintenance and battery replacement as needed.

The electronic circuitry 1203 may be powered by a rechargeable micro-battery integrated within the prosthetic eye. Additionally, a wireless charging coil may be embedded in the structure to allow inductive charging through an external device. The circuitry may include Bluetooth or Wi-Fi communication modules, enabling the prosthetic eye to transmit real-time sensory data to an external device, such as a smartphone or assistive device.

Following the insertion of the electronic circuitry 1203, a second layer of polymerizable mixture 1204 may be deposited to encapsulate the components securely. This deposition may be performed in a drop-by-drop or layer-by-layer droplet deposition manner, providing a firm yet accessible enclosure for the circuitry. The material composition may include protective coatings to shield the circuitry from external moisture and mechanical stress.

At Step 4, a third deposition cycle of polymerizable mixture 1205 may be performed to create an internal cavity 1206 within the prosthetic eye. This cavity may serve multiple functional purposes, such as housing fluid reservoirs, accommodating thermal expansion of liquid components, or providing a chamber for active cooling of electronic components.

The internal cavity 1206 may also function as a shock-absorbing buffer, reducing stress on the integrated electronic components and enhancing the durability and lifespan of the prosthetic eye. The cavity may be sealed using a semi-permeable membrane, allowing the controlled exchange of fluids or gases between different compartments.

In some embodiments, the final deposition cycle 1205 may involve the application of surface texturing techniques to simulate the natural appearance of the eye. This may include printed vein patterns, pigmentation layers, and high-resolution textures that replicate the fine details of the sclera and iris.

At Step 5, the internal cavity 1206 may be filled with a liquid solution 1207, which acts as a suspension medium for the iris 1208, thereby enabling its natural movement within the prosthetic eye 1220. The liquid solution 1207 may be selected based on its optical clarity, viscosity, and long-term stability, preventing sedimentation or clouding over time. In some embodiments, the liquid solution 1207 may comprise a saline-based solution similar to natural tear fluids, allowing the iris 1208 to move freely while maintaining a realistic refraction of light. In other embodiments, the liquid solution 1207 may include a low-viscosity silicone oil, providing enhanced damping effects, which regulate the speed and smoothness of the iris movement when the prosthetic eye 1220 may be subjected to tilt or acceleration forces. Additionally, in certain implementations, magnetically reactive liquid compositions may be employed, allowing the movement of the iris 1208 to be controlled electromagnetically in advanced prosthetic eye models. The introduction of the liquid solution 1207 into the internal cavity 1206 may be performed using precision dispensing nozzles, minimizing bubble formation within the cavity, which can otherwise distort the visual aesthetics of the prosthetic eye 1220.

At Step 6, the iris 1208 may be introduced into the internal cavity 1206, where it becomes suspended within the liquid solution 1207, allowing it to shift freely in response to gravitational forces, movement, and head orientation. The iris 1208 may be 3D-printed with high-resolution additive manufacturing techniques, facilitating that its coloration and fiber structure accurately replicate a natural human iris. In some embodiments, the iris 1208 may be fabricated using photochromic materials, allowing for adaptive light sensitivity, meaning that its pigmentation may darken in bright environments and lighten in dim environments, mimicking natural pupil dilation. Additionally, the diameter of the iris 1208 is precisely designed to be slightly smaller than the internal cavity 1206, providing free movement without excessive displacement, which may otherwise cause unrealistic shifting. The integration of the movable iris 1208 significantly enhances the realism of the prosthetic eye 1220, making it visually indistinguishable from a biological eye in certain lighting conditions. Furthermore, in advanced prosthetic applications, the iris 1208 may be embedded with microsensors that track external light intensity and adjust the shading dynamically.

At Step 7, a transparent membrane 1209 is carefully applied over the internal cavity 1206, effectively sealing the iris 1208 and liquid solution 1207 within the prosthetic eye 1220. The transparent membrane 1209 serves multiple functions, primarily protecting the internal components while allowing optical clarity for realistic light reflection and refraction. The transparent membrane 1209 may be fabricated using biocompatible polymer films, such as polyurethane, polydimethylsiloxane (PDMS), or silicone-based hydrogels, which possess flexibility and self-healing properties, thereby increasing the durability of the prosthetic eye 1220. The bonding of the transparent membrane 1209 to the surrounding structure may be achieved using thermal curing, UV-curable adhesives, or chemical vapor deposition, providing a strong yet flexible connection that maintains the fluid integrity within the internal cavity 1206. In some embodiments, the surface of the transparent membrane 1209 may be treated with anti-glare coatings, preventing unwanted reflections that may make the prosthetic eye 1220 appear artificial under certain lighting conditions. Additionally, micro-texturing techniques may be employed on the surface of the transparent membrane 1209, enabling better interaction with natural tear films, which further contributes to the realistic appearance of the prosthetic eye 1220.

At Step 8, a final polymerization layer 1210 may be applied over the transparent membrane 1209, further enhancing the durability, texture, and realism of the prosthetic eye 1220. This final layer 1210 is selectively deposited layer-by-layer or drop-by-drop, providing precise shaping of the scleral region. The deposition of the final polymer layer 1210 also allows for the integration of vein patterns 1211, which are important for matching the prosthetic eye 1220 to a user's natural eye aesthetics. The vein patterns 1211 may be fabricated using multi-material printing techniques, wherein red-tinted biocompatible inks or polymers are layered onto the sclera to resemble real blood vessels. In some implementations, the vein patterns 1211 may be customized based on a scan of the user's natural eye, facilitating that the prosthetic eye 1220 maintains a personalized appearance that seamlessly blends with surrounding ocular tissues.

As the final polymerization cycle (1210) completes, the prosthetic eye 1220 is fully formed, comprising:
An internal cavity 1206, providing a space for the free-floating iris 1208.
A liquid solution 1207, maintaining optical clarity and iris movement.
A 3D-printed iris 1208, capable of naturalistic movement and light adaptation.
Liquid pouches 1202A-1202B, designed for hydration and medication delivery.
Electronic circuitry 1203, enabling advanced functionalities such as motion tracking, wireless communication, and AI-driven enhancements.
A transparent membrane 1209, serving as a protective and optically clear layer.
Vein patterns 1211, creating a realistic scleral appearance.

In some embodiments, the final prosthetic eye 1220 may also incorporate sensor arrays within the scleral structure, allowing the device to track eye movement, detect pressure changes, or integrate with neural prosthetics for enhanced functionality in medical or research applications. Additionally, specialized coatings on the prosthetic eye 1220 may include UV-blocking layers, antimicrobial properties, or hydrophobic surface treatments, further increasing usability and comfort for the wearer.

The fabrication of the prosthetic eye 1220 via multi-stage additive manufacturing enables high levels of customization, rapid prototyping, and material optimization, making the process suitable for mass production as well as bespoke ocular prosthetics tailored to individual patients. The completion of Step 8 marks the final stage of the additive printing process, after which post-processing steps, such as surface finishing, sterilization, and functional testing, may be performed to prepare the prosthetic eye 1220 for real-world use.

Through these integrative design principles and novel manufacturing approaches, the prosthetic eye 1220 presents a breakthrough in ocular prosthetics, offering not only aesthetic enhancements but also functional benefits, thereby improving the quality of life for users requiring advanced ocular replacements.

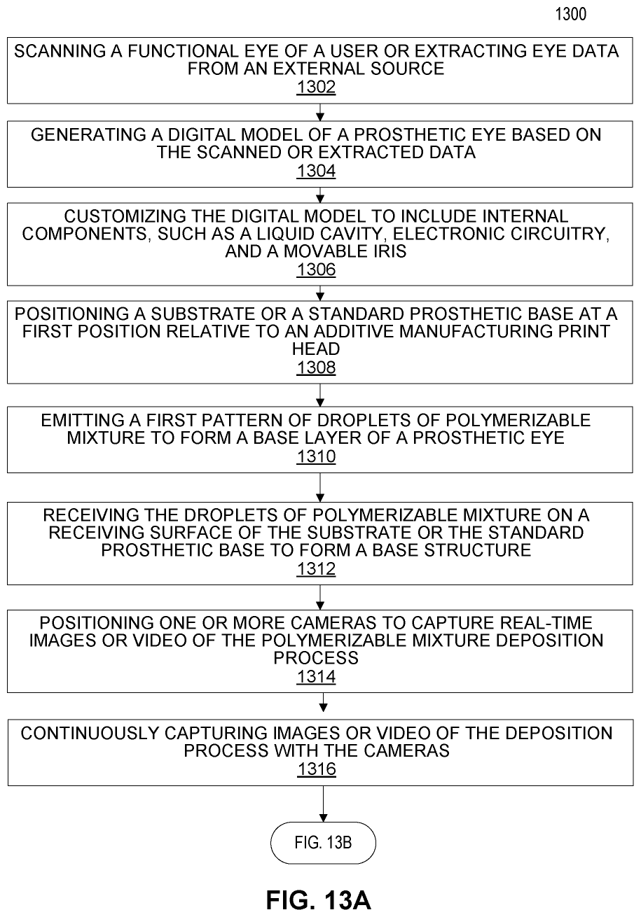

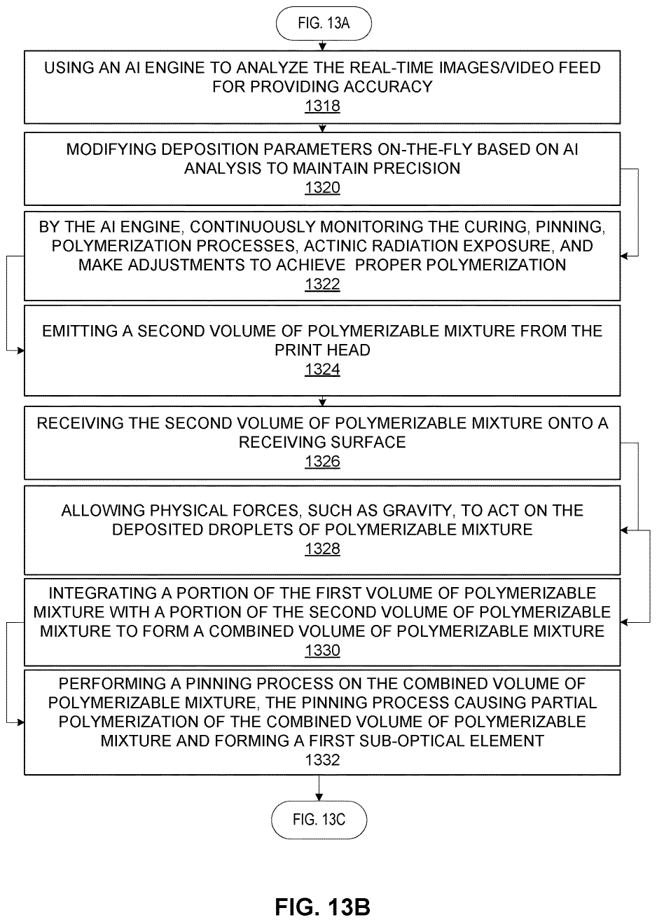

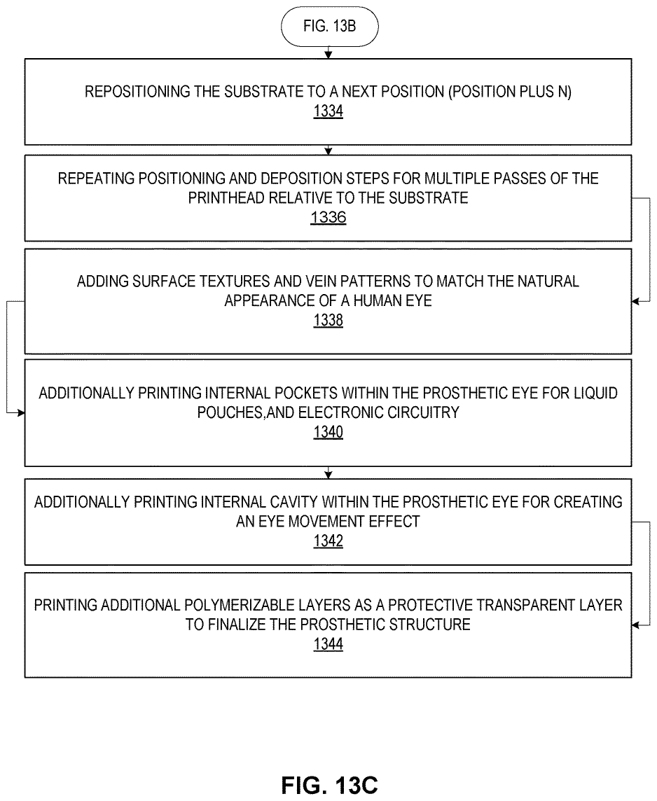

Referring now to FIGS. 13A, 13B, and 13C, a flowchart 1300 is illustrated, depicting exemplary method steps 1302-1344 that may be executed in some embodiments of the present invention to fabricate a prosthetic eye using additive manufacturing techniques. The flowchart 1300 comprises a structured methodology for obtaining eye data, generating a digital model of a prosthetic eye, customizing the model with internal components, and employing additive manufacturing for the controlled deposition of polymerizable mixture to build the prosthetic eye layer by layer using patterned droplets. The method may further include real-time monitoring of the deposition process using one or more cameras, allowing for quality control and precision adjustments during fabrication. The described process may particularly be advantageous for generating personalized prosthetic eyes that accurately replicate the aesthetic and functional aspects of a user's original eye. The method may be implemented using various digital scanning technologies, 3D printing systems, and real-time imaging techniques to achieve a highly detailed and customized ophthalmic device.

At step 1302, the process begins by scanning a functional eye of a user or extracting eye data from an external source. This step may involve non-invasive scanning technologies such as optical coherence tomography (OCT), structured light scanning, or photogrammetry to capture detailed three-dimensional (3D) surface data of the user's existing functional eye. In cases where the user has lost an eye, reference data may be extracted from high-resolution images, medical imaging databases, or artificial intelligence (AI)-assisted reconstructions of a previously captured image of the user's eye. The extracted data may include iris color, scleral vein patterns, corneal curvature, and other anatomical details that contribute to a natural and realistic prosthetic eye design.

At step 1304, a digital model of a prosthetic eye may be generated based on the scanned or extracted data. The digital model may be developed using computer-aided design (CAD) software or AI-powered image reconstruction techniques to replicate the structure, color, and proportions of the original eye. The model may include surface textures and transparency variations to simulate the complex visual characteristics of a human eye. In some embodiments, the digital model may be adjusted to correct any distortions or imperfections introduced during scanning. The model may also allow for modifications to accommodate additional functional elements such as liquid reservoirs, embedded electronics, or a movable iris.

At step 1306, the digital model is further customized to include internal components such as a liquid cavity, electronic circuitry, and a movable iris. The customization process involves defining separate sections within the prosthetic eye where additional features may be integrated. For example, a liquid cavity may be designed to hold a biocompatible solution that enhances comfort and provides hydration for extended wear. Electronic circuitry may be embedded to facilitate visual feedback mechanisms, sensor integrations, or wireless communication with external devices. A movable iris may be incorporated to simulate natural eye movement, providing an added level of realism for users who seek a more lifelike prosthesis.

At step 1308, a substrate or a standard prosthetic base may be positioned at a first position relative to an additive manufacturing print head. The substrate serves as a foundational support upon which the polymerizable mixture will be deposited to form the prosthetic eye. In some embodiments, the substrate may be a flat surface designed to temporarily hold the printed layers before the final prosthetic eye is removed and assembled. Alternatively, a pre-existing prosthetic base may be used as a starting point for fabrication, allowing the additive manufacturing process to build upon an existing structure. The positioning may be precisely controlled using automated alignment mechanisms to maintain accuracy throughout the printing process.

At step 1310, the additive manufacturing print head emits a first pattern of droplets of polymerizable mixture to form a base layer of the prosthetic eye. The deposition process involves precisely timed ejections of polymer droplets, which solidify upon contact with the substrate or previously deposited layers. The first pattern may define the foundational shape and curvature of the prosthetic eye, facilitating that subsequent layers align with the intended anatomical structure. The polymerizable mixture may include specialized compounds that enhance flexibility, durability, and optical clarity, contributing to the overall performance of the final prosthetic device.

At step 1312, the deposited droplets of polymerizable mixture are received on the receiving surface of the substrate or the standard prosthetic base to form a base structure. The receiving surface may be pre-treated with adhesion promoters or textured coatings to facilitate proper bonding between layers. As the droplets land on the surface, they may spread and merge to create a seamless transition between printed sections. The base structure acts as a stabilizing foundation upon which additional layers and components may be added in subsequent steps. In some embodiments, the thickness and density of the deposited material may be adjusted dynamically to optimize mechanical properties.

At step 1314, one or more cameras are positioned to capture real-time images or video of the polymerizable mixture deposition process. These cameras may be integrated within the additive manufacturing system or externally mounted to provide different viewing angles. High-resolution imaging allows for continuous monitoring of the printing process, detecting any inconsistencies or defects in the deposited material. In some embodiments, the captured images may be analyzed by AI-driven algorithms to make real-time adjustments to the print parameters, facilitating that each layer conforms to the desired specifications. The ability to visually inspect the deposition process provides an added level of quality control, reducing the likelihood of errors.

At step 1316, the cameras continuously capture images or video of the deposition process as the prosthetic eye is built layer by layer using patterned droplets. This continuous monitoring allows for the identification of irregularities such as material deformation, incomplete layer bonding, or excessive droplet spreading. If an issue is detected, corrective measures may be implemented immediately, such as adjusting deposition speed, modifying curing parameters, or introducing additional stabilization layers. In some embodiments, the captured video may also be archived for post-production analysis, enabling researchers or manufacturers to refine the additive manufacturing process based on observed patterns and improvements.

At step 1318, an AI engine may be utilized to analyze real-time images or video feeds captured during the deposition of the polymerizable mixture. The AI engine may process the visual data in real time, detecting variations in deposition patterns, inconsistencies in layer bonding, or deviations from the pre-defined structural model of the prosthetic eye. The AI engine may be configured to perform edge detection, layer thickness measurements, and anomaly detection based on machine learning models trained with prior fabrication data. This analysis may contribute to optimizing the quality of the deposited layers and predicting any potential defects that may arise in subsequent printing steps. For example, if the AI detects insufficient material flow in a particular region, it may trigger corrective actions, such as adjusting the deposition speed, modifying nozzle pressure, or recalibrating the print head to compensate for detected irregularities.

At step 1320, the deposition parameters are modified dynamically based on the AI analysis to maintain precision. The AI engine may communicate directly with the print head control system, adjusting various printing parameters, including droplet size, ejection velocity, and layer curing intervals. The AI-driven modifications allow the system to compensate for environmental factors such as temperature variations, humidity levels, and material viscosity fluctuations, which may otherwise impact the consistency of the deposited layers. For example, if the AI detects a slight over-deposition of material in a specific area, it may adjust the subsequent layer thickness to counterbalance the accumulation and maintain structural accuracy. These real-time adjustments enable the fabrication process to achieve a high degree of precision, reducing the need for post-processing modifications.

At step 1322, the AI engine continuously monitors the curing, pinning, and polymerization processes, as well as actinic radiation exposure, and makes adjustments to achieve proper polymerization. In some embodiments, sensors embedded in the printing environment may relay data to the AI engine regarding the intensity and duration of actinic radiation exposure applied to the polymerizable mixture. Based on this data, the AI may fine-tune the curing schedule, preventing under-curing or over-curing of material. The AI may also track polymerization kinetics, facilitating that each layer achieves the desired degree of cross-linking before subsequent layers are deposited. This active monitoring mitigates issues such as differential shrinkage, mechanical stress accumulation, or localized material degradation, which may affect the final performance of the prosthetic eye.

At step 1324, a second volume of polymerizable mixture may be emitted from the print head onto the partially formed structure of the prosthetic eye. The second volume may contain a compositionally modified polymerizable mixture tailored to enhance specific properties, such as elasticity, light transmission, or surface texture. In some embodiments, the second volume may include functional additives, such as UV stabilizers, biocompatible lubricants, or refractive index modifiers, to improve the visual and mechanical characteristics of the final prosthetic eye. The print head may be configured to deposit the second volume in a controlled pattern, following the contours of the previously deposited material to maintain uniformity in layer thickness and optical performance.

At step 1326, the second volume of polymerizable mixture is received onto a designated receiving surface (e.g., a surface created by the first volume of polymerizable mixture), which may be an intermediate layer of the prosthetic eye or a structural support element. Upon deposition, the second volume may spread across the receiving surface due to surface tension and capillary effects, forming a thin, continuous film that integrates seamlessly with underlying layers. In some embodiments, the receiving surface may be pre-treated with a compatibility-enhancing agent to promote adhesion and prevent delamination between deposited layers. The receiving step is important for defining the final geometry of the prosthetic eye and facilitating that each material layer interacts as intended within the additive manufacturing system.

At step 1328, physical forces, such as gravity, are allowed to act on the deposited droplets of polymerizable mixture, influencing their final shape and distribution. Gravity-driven flow may assist in leveling out surface irregularities, filling microscopic voids, and achieving a uniform layer thickness. The system may leverage controlled deposition angles to guide the material's natural spreading behavior, facilitating that the final surface profile remains consistent with the intended design. In some embodiments, additional post-deposition stabilization techniques, such as electrostatic manipulation or surface energy control, may be employed to enhance the self-leveling properties of the polymerizable mixture.

At step 1330, a portion of the first volume of polymerizable mixture may be integrated with a portion of the second volume to form a combined volume of polymerizable mixture. The intermixing of these volumes may occur at the molecular level, facilitated by diffusion, polymer chain entanglement, and cross-linking reactions. The degree of integration may be controlled by adjusting deposition time intervals, temperature settings, and chemical composition. This controlled integration allows for seamless bonding between layers while preserving distinct functional properties, such as optical clarity in the iris region and increased mechanical strength in structural regions.

At step 1332, a pinning process may be performed on the combined volume of polymerizable mixture to induce partial polymerization. The pinning process involves exposing the material to a calibrated dose of actinic radiation, initiating a controlled polymerization reaction without fully curing the material. This step may particularly be important for maintaining flexibility in subsequent processing steps while facilitating that deposited layers achieve sufficient cohesion. The partial polymerization stage also serves to lock in the desired optical properties of the prosthetic eye, preventing unwanted diffusion or phase separation of functional additives. By carefully managing this process, the fabrication system achieves a balance between structural integrity and modifiability, allowing further refinement before final curing.

At step 1334, the substrate may be repositioned to a next position (Position Plus N) relative to the additive manufacturing print head. This repositioning step is executed with precision to align subsequent depositions of polymerizable material accurately over previously printed layers. The movement of the substrate may be controlled through stepper motors, linear actuators, or robotic arms that are programmed to shift the substrate in pre-defined increments. The repositioning step is vital to the layer-by-layer fabrication process, as it facilitates that all structural and functional components of the prosthetic eye are built progressively with seamless integration between material layers. The alignment of the substrate is continuously monitored through feedback systems, such as laser positioning sensors, camera-based tracking, or AI-assisted calibration routines, to prevent errors in deposition.

At step 1336, the positioning and deposition steps are repeated for multiple passes of the print head relative to the substrate. These repeated passes enable the gradual formation of the prosthetic eye structure, allowing for precision in material layering and controlled polymerization kinetics. The iterative deposition of polymerizable material may include alterations in print parameters such as droplet size, deposition rate, and actinic radiation exposure, which are dynamically adjusted based on real-time process feedback. Each successive pass may contribute to enhancing the structural integrity of the prosthetic eye by reinforcing existing layers and integrating additional functional elements. The repeated deposition steps also allow the gradual transition between different material compositions, such as shifting from a rigid scleral portion to a flexible iris region, to accurately mimic the mechanical properties of a natural human eye.

At step 1338, surface textures and vein patterns are added to match the natural appearance of a human eye. The incorporation of vein-like patterns in the scleral region of the prosthetic eye may be achieved through specialized additive manufacturing techniques such as multi-material jetting, laser-assisted surface texturing, or pigment-based deposition. The vein patterns may be designed using digital imaging techniques that replicate real human eyes, facilitating that the aesthetic properties of the prosthetic eye are indistinguishable from a biological counterpart. These textures not only contribute to visual realism but also provide a tactile feel that may be beneficial for handling and fitting the prosthetic eye within an ocular socket. Additionally, variations in texture may be introduced to simulate the microscopic irregularities of the human sclera, further enhancing the authenticity of the prosthetic eye.

At step 1340, internal pockets within the prosthetic eye are additionally printed for liquid pouches and electronic circuitry. These internal pockets may be precisely structured to accommodate reservoirs of fluid used for hydration, lubrication, or controlled release of ophthalmic medication. The dimensions and positioning of these pockets are carefully engineered to prevent interference with the other functional components of the prosthetic eye while allowing for controlled dispensing of liquid when necessary. The inclusion of electronic circuits within these pockets may facilitate functionalities such as embedded cameras, wireless communication modules, sensors for detecting ocular pressure, or micro-actuators for enhancing the movement of the iris. The internal pockets may be sealed or fitted with micro-valves to regulate the flow of liquid, facilitating that the prosthetic eye remains comfortable for extended wear while maintaining its visual and functional properties.

At step 1342, an internal cavity is additionally printed within the prosthetic eye to create an eye movement effect. The internal cavity is designed to allow the iris component of the prosthetic eye to exhibit controlled movement, simulating the natural dynamics of a biological eye. This cavity may be filled with a specialized fluid medium that facilitates smooth motion of the iris by reducing friction and providing a buoyant effect. The design of the cavity may incorporate channels or guiding structures that define the range of motion of the iris, preventing unintended shifts that may disrupt the visual alignment of the prosthetic eye. The interaction between the internal cavity, the surrounding fluid, and the freely suspended iris component may be optimized through computational fluid dynamics simulations to refine the motion characteristics of the prosthetic eye under different gaze orientations.

At step 1344, additional polymerizable layers are printed as a protective transparent layer to finalize the prosthetic structure. This final protective layer serves multiple purposes, including safeguarding the internal components from environmental contaminants, providing structural reinforcement, and enhancing the optical clarity of the prosthetic eye. The transparent layer may be composed of a specialized biocompatible material that offers high resistance to wear and tear while maintaining a refractive index similar to that of a natural human eye. In some embodiments, the protective layer may be treated with anti-glare coatings, UV-resistant additives, or hydrophobic surface treatments to enhance durability and comfort. The deposition of this final layer may involve controlled curing processes to achieve an ultra-smooth finish, eliminating any imperfections that may impact the optical performance of the prosthetic eye.

In some variants of the present invention, a control command may be used to determine how many passes a 3D printhead has made over a receiving surface. A number of passes of the 3D printhead may correlate with a thickness of polymerizable mixture deposited and also correlate with an amount of energy intensity at particular locations of an energy intensity pattern of deposited polymerizable mixture. In this manner, deposited polymerizable mixture may be deposited, pinned and ultimately cured in a shape and volume suitable to form a prosthetic eye with desired qualities. Deposited polymerizable mixture achieves sufficient thickness and suitable shape by repeated application of a corresponding grayscale image.

In some variants of the present invention, each pass of the 3D printhead may print a pattern of polymerizable mixture corresponding with a same grayscale image, in other embodiments, a different pass of the 3D printhead may correspond with a different grayscale image than a previous pass. As mentioned previously, in some variants of the present invention, each pass of a 3D printhead depositing polymerizable mixture may be followed by exposures of the polymerizable mixture to a gelation process, such as an amount of actinic radiation, thermal activation, or the like, that is sufficient to partially polymerize the deposited polymerizable mixture. A curing process may be performed after a final pass of polymerizable mixture is performed. In various embodiments, a final layer may be exposed to a pinning step, and/or move directly to a full curing process.

Curing and/or pinning may be facilitated by inclusion of one or more photo initiators with the deposited monomer. Photoinitiators may include, by way of non-limiting example, initiators activated by energy of or about 392 nM and 400 nM.

In some examples, an energy intensity pattern (e.g., a grayscale image) may be derived from an article in physical form that is processed, such as via an optical scanning process, image capture process, or photographing process, to capture the energy intensity data into an electronic form, such as, for example, a digital data value, and the electronic form may be converted to control commands.

Some variants of the present invention include a grayscale image with a generally spherical shape, where the lighter values are associated with thicker deposits. Thus, a different conversion protocol may be assigned to different grayscale images depending on which values correspond to thick and thin deposits, respectively.

In some examples, a single grayscale image may be used to represent a desired prosthetic eye and its associated control commands. The single grayscale image is repeatedly deposited in successive passes of the 3D printhead, one pass following another until a desired optical quality is embodied in the deposited polymerizable mixture which may be cured to form an article that also meets physical parameters suitable for wearing on an eye of a patient.

In other examples, a series of grayscale images may be assembled to create multiple sets of control commands. The control commands may result in deposit of different shape designs and physically create an additive composite of the images. In other examples, multiple grayscale images may be combined and processed before any processing occurs. In some examples, a combination of multiple images may be normalized to correspond with upper and lower thickness factors.

In some examples, different features such as edge profiles 303, alignment features, and the like, may be programmed into an optical element command protocol by the addition of grayscale images to a prosthetic eye profile.

In some examples, a grayscale image may be referenced from numerous filetypes such as, by way of non-limiting example, one or more of: jpeg, tiff, bmp, png and the like, may be used to create a control command protocol to print a desired article, such as a prosthetic eye, by varying an amount of polymerizable mixture that is deposited at disparate locations thereby resulting in with more polymerizable mixture deposition in areas targeted for thicker deposits. The result of printing an entire pattern may result in an article with no interstitial areas.

In some variants of the present invention, a grayscale in an image or additive combination of images, may correspond to processing of multiple disparate passes of a 3D printhead, wherein an amount of polymerizable mixture deposited in specific locations and subjected to a pinning process before a next deposition pass is completed. A polymerizable mixture deposited during a printing process pass may be a monomer mixture with various included photo initiators. One of the photo initiators may be associated with a wavelength of actinic radiation exposure during an associated pinning processes. After multiple printing passes have been processed, an entire volume of polymerizable mixture deposited on a receiving substrate may be subjected to a curing process. In some examples, the curing process may be exposure to actinic radiation of a different wavelength and exposure time, intensity and the like.

In another aspect, in some embodiments, a grayscale pattern or an energy intensity pattern may be dithered via a dithering process or algorithm prior to the generation of a control command for the printhead based upon the grayscale or energy intensity pattern in order to generate a smoother image deposited via expulsion of droplets from the printhead and accumulation prior to curing. Dithering may include, way of non-limiting examples, processes consistent with Floyd-Steinberg, Burkes, Sierra, Two Row Sierra, Jarvis, Stevenson, Arce, or other processes.

### General Remarks

Although the present description and claims occasionally refer to a mixture (such as a polymerizable mixture), an initiator, or other additives, it is within the scope of this invention that the materials and compositions defined herein may comprise one, two, or more types of individual constituents. In such embodiments, a total amount of a respective constituent should correspond to an amount defined above for the individual constituent.

The(s) in the expressions: mixture(s), initiator(s), etc. indicates that one, two, or more types of the individual constituents may be present. On the other hand, when the expression one is used, only one (1) of the respective constituent is present.

It should be understood that the expression % means the percentage of the respective component by weight, unless otherwise noted.

### Conclusion

A number of embodiments of the present disclosure have been described. While this specification contains many specific implementation details, there should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, separation of various system components in the embodiments described above should not be understood as requiring such separation in all embodiments, and it should be understood that the described program components and systems can generally be integrated together in a single hardware and/or software product or packaged into multiple products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A method of forming a prosthetic lens, comprising the steps of:
a.) generating a digital model of a desired configuration of at least an iris and/or pupil for a patient, wherein said digital model substantially replicates coloring of the desired at least iris and/or pupil;
b.) using the digital model to program one or more additive manufacturing printheads to:
deposit a first volume of polymerizable mixture in a predetermined pattern onto a receiving surface of a substrate as a first volume of deposited droplets, and
deposit at least a second volume of polymerizable mixture in a predetermined pattern onto said substrate and/or said first volume of deposited droplets;
c.) integrating a portion of the first volume and at least second volume to form a combined volume of polymerizable mixture;
d.) pinning the combined volume of polymerizable mixture to cause partial polymerization of the combined volume of polymerizable mixture;
e.) repeating steps b.) through d.) until the successive deposited volumes of polymerizable mixture substantially replicates the digital model; and
f.) fully curing all polymerizable mixture,
wherein one or more volumes of polymerizable mixture deposited include one or more embedded pigments that collectively substantially replicate the coloring of the desired iris and/or pupil, and
wherein at least the last successive deposited volume of polymerizable mixture is transparent.

2. The method according to claim 1 wherein the prosthetic lens is a cosmetic lens.

3. The method according to claim 1, wherein the prosthetic lens has a size and shape suitable for use with a prosthetic base to form a prosthetic eye for said patient.

4. The method according to claim 3, wherein the substrate is the prosthetic base.

5. The method according to claim 1, wherein the digital model further includes a desired configuration of a sclera.

6. The method according to claim 1, wherein the digital model includes anatomical and optical details including a size, a shape and a color of the iris, pupil dimensions and location, and curvature of the desired prosthetic lens.

7. The method according to claim 6, wherein the anatomical and optical details are derived from one of a patient's eyes.

8. The method according to claim 1, further comprising obtaining an image of a desired eye to generate the digital model.

9. The method according to claim 8, wherein the prosthetic lens is for a patient, and the desired eye is the patient's other eye.

10. The method according to claim 8, wherein the desired eye is a third party's eye, and the image is an existing image of the third party's eye.

11. The method according to claim 8, wherein the at least one image is an existing image of an eye.

12. A prosthetic lens comprising:
an iris portion; and
a pupil portion surrounded by said iris portion,
wherein said iris and pupil portions are comprised of a plurality of layers of polymerizable mixture, wherein at least one of said plurality of layers includes one or more embedded pigments that collectively provide color to said iris and pupil portions, and wherein at least a last one of said layers is transparent.

13. The prosthetic lens according to claim 9, further comprising a scleral portion surrounding said iris portion.

14. The prosthetic lens according to claim 9, wherein said prosthetic lens is a cosmetic lens.

15. The prosthetic lens according to claim 9, wherein the prosthetic lens has a size and shape suitable for use with a prosthetic base to form a prosthetic eye for said patient.

16. The prosthetic lens according to claim 9, wherein the iris and pupil portions substantially replicate an iris and pupil of an eye of a patient.

17. A method of forming an ophthalmic prosthesis, comprising:
generating a digital model of a desired configuration for the ophthalmic prosthesis, wherein the digital model includes at least an iris portion and a pupil portion;
programming one or more additive manufacturing printheads according to the digital model;
positioning a substrate relative to the one or more additive manufacturing printheads;
depositing, with the one or more additive manufacturing printheads according to the digital model, a polymerizable mixture in successive predetermined patterns onto a receiving surface of the substrate and/or a previously deposited polymerizable mixture;
integrating at least a portion of a successive deposited polymerizable mixture to form a combined volume of polymerizable mixture;
pinning the combined volume of polymerizable mixture to cause partial polymerization;
repeating the depositing, integrating, and pinning until the deposited polymerizable mixture substantially replicates the digital model; and
fully curing the polymerizable mixture to form the ophthalmic prosthesis,
wherein one or more deposited volumes include one or more embedded pigments that collectively provide color to the iris portion and the pupil portion, and
wherein at least a final deposited volume is transparent.

18. The method according to claim 17 wherein the ophthalmic prosthesis is a cosmetic lens.

19. The method according to claim 17, wherein the ophthalmic prosthesis has a size and shape suitable for use with a prosthetic base to form a prosthetic eye for a patient.

20. The method according to claim 17, wherein the digital model includes anatomical and optical details comprising iris size, iris shape, iris color, pupil dimensions, pupil location, and curvature of the ophthalmic prosthesis.
